(19) United States
(12) Patent Application Publication
(10) Pub. No.: US 2013/0116219 A1
(43) Pub. Date: May 9, 2013

(54) ANTIMICROBIAL CARBOLINE COMPOUNDS

(71) Applicant: ALCON RESEARCH, LTD., Fort Worth, TX (US)

(72) Inventors: David Archer Ellis, Fort Worth, TX (US); Suchismita Mohapatra, Arlington, TX (US); Abdelmoula Namil, Arlington, TX (US); Hwang-Hsing Chen, Fort Worth, TX (US); Byron Severns, Arlington, TX (US); David B. Belanger, Lexington, MA (US)

(73) Assignee: ALCON RESEARCH, LTD., Fort Worth, TX (US)

(21) Appl. No.: 13/668,073

(22) Filed: Nov. 2, 2012

Related U.S. Application Data
(60) Provisional application No. 61/555,584, filed on Nov. 4, 2011.

(51) Int. Cl. A61K 31/437; A61K 31/5377; A61K 31/4545; A61K 31/541; A61K 31/444; A61K 31/573; A61K 31/496; A61P 27/02; A61P 31/00; A61P 29/00; A61K 31/56; C07D 471/04; A61K 31/506

(52) U.S. Cl. 514/171; 546/85; 514/292; 544/126; 514/232.8; 544/58.6; 514/228.2; 514/210.21; 514/210.18; 544/331; 514/275; 544/361; 514/249

(57) ABSTRACT

The invention provides compositions comprising carboline compounds for treating infections such as viral conjunctivitis. The invention also provides methods for treating of other infections, including ocular infections. More particularly, the present invention relates to compositions comprising carboline compounds for the treatment of ocular infections such as viral conjunctivitis, particularly those caused by adenovirus

### CROSS-REFERENCE TO RELATED APPLICATION

This application claims priority under 35 U.S.C. §119 to U.S. Provisional Patent Application No. 61,555,584 filed Nov. 4, 2011, the entire contents of which are incorporated herein by reference.

### TECHNICAL FIELD OF THE INVENTION

The present invention generally relates to carboline compounds useful for the treatment of infections, and specifically to methods and compounds for the treatment of infections caused by microbes such as adenovirus.

### BACKGROUND OF THE INVENTION

Viral conjunctivitis represents a significant, unmet medical need with no available treatment options. Although a number of viral species can initiate a conjunctival infection, adenovirus (AdV) accounts for the largest number of clinical cases. Ocular adenoviral infections are highly contagious, easily spreading from infected to non-infected eye, and routinely passed to family members. As a result, over 3 million missed school days per year in the U.S. are associated with adenoviral infections. Adenoviral-associated follicular conjunctivitis is relatively mild and self-limiting, persisting for two to four weeks. Specific serotypes associated with follicular adenoviral conjunctivitis include Ad3, Ad4, and Ad7. In contrast, serotypes Ad8, Ad19, and Ad37 are associated with epidemic keratoconjunctivitis, a highly contagious and more serious disease involving the cornea and conjunctiva, with potential long-term visual acuity consequences. Thus, there is an acute need for an effective anti-adenoviral agent with broad spectrum serotype activity.

In the last 20 years, significant progress has been made in the treatment of herpes simplex virus (HSV), influenza virus, hepatitis B virus (HBV), and human immunodeficiency virus (HIV) infections. However, as noted above, no treatment is available for AdV infection. Recently, Harvey et al. (Antiviral Res. 82:1-11, 2009) described a tetrahydrocarbazole, GSK-983, that exhibited antiviral activity. GSK-983 inhibited the replication of AdV serotype 5 (Ad5), polyoma virus SV40, human papillomavirus (HPV), and Epstein-Barr virus (EBV) with EC₅₀ values in the 5-20 nM range. However, GSK-983 has not previously demonstrated activity against AdV serotypes found in ophthalmic infections. Accordingly, there is a need for compounds useful in the treatment of infection caused by microbes such as adenovirus.

### BRIEF SUMMARY OF THE INVENTION

The present invention relates to methods and compounds for the treatment of infections caused by microbes such as bacteria, virus, fungi etc., and particularly for the treatment of ocular viral infections such as those caused by adenovirus. Such ocular viral infections include, but are not limited to, viral conjunctivitis, (including adenoviral conjunctivitis), follicular conjunctivitis, ocular keratitis and epidemic keratoconjunctivitis. Compounds of the present invention have been unexpectedly found to possess broad spectrum antiviral activity against adenovirus (AdV) serotypes commonly found in ocular AdV infections. Compounds of the present invention have also demonstrated high aqueous solubility at acidic pH compared to other compounds known in the art.

Another embodiment of the present invention contemplates an ophthalmic pharmaceutical composition useful in the treatment of ocular infection, comprising an effective amount of a compound according to Formulas I-III.

Yet another embodiment of the present invention comprises a method of treating ocular infection comprising administering a therapeutically effective amount of an ophthalmic pharmaceutical composition, where the composition comprises an effective amount of a compound according to Formulas I-III.

Still another embodiment of the present invention is an ophthalmic pharmaceutical composition useful in the treatment of ocular infection comprising an effective amount of Compounds 1-110.

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention when considered in connection with any accompanying figures. However, figures provided herein are intended to help illustrate the invention or assist with developing an understanding of the invention, and are not intended to be definitions of the invention's scope.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a graph showing the results of an evaluation of the antiviral activity of Compound 6 of the present invention in a rabbit model of adenoviral ocular keratitis.

### DETAILED DESCRIPTION OF THE INVENTION

In one embodiment, the present invention provides a compound of Formula I:

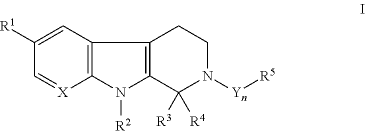

where:

X=C or N;

Yₙ=bond, CH₂, C(O), C(O)O, C(O)NR⁶, or SO₂;

n=0 or 1;

p=0, 1 or 2;

R¹=H, halogen, alkyl, nitrile or amide;

R²=H or alkyl;

R³ and R⁴ are independently selected from H, alkyl, cycloalkyl, heteroalkyl, heterocycloalkyl, aryl, or heteroaryl, or R³ and R⁴ can form a 3- to 6-membered cycloalkyl or heterocycloalkyl ring optionally substituted with alkyl or halogen;

R⁵=R⁷N(R⁸)R⁹, R⁷C(O)N(R⁸)R⁹, or R⁷SO₂R¹⁰

R⁶=H or alkyl;

R⁷=null or optionally substituted alkylene;

R⁸ and R⁹ are independently selected from H, alkyl, heteroalkyl, cycloalkyl, heterocycloalkyl, aryl, or heteroaryl each of which can be optionally substituted or R⁸ can combine with R⁷ and/or R⁹ to form a 3- to 8-membered heterocycle optionally substituted with halogen, alkyl, cyano, NR¹⁰, and/or S(O)ₚ; and

R¹⁰=H or alkyl.

In another embodiment, the present invention provides a compound of Formula II:

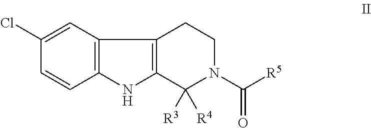

where:

R³ and R⁴ are independently selected from H, alkyl, cycloalkyl, heteroalkyl, heterocycloalkyl, aryl, or heteroaryl, or R³ and R⁴ can form a 3- to 6-membered cycloalkyl or heterocycloalkyl ring optionally substituted with alkyl or halogen;

R⁵=R⁷N(R⁸)R⁹, R⁷C(O)N(R⁸)R⁹, or R⁷SO₂R¹⁰

R⁷=optionally substituted alkylene;

R⁸ and R⁹ are independently selected from H, alkyl, heteroalkyl, cycloalkyl, heterocycloalkyl or heteroaryl each of which can be optionally substituted or R⁸ can combine with R⁷ and/or R⁹ to form a 4- to 8-membered heterocycle optionally substituted with halogen, alkyl, NR¹⁰, N(O) and/or S(O)ₚ; and

R¹⁰=H or alkyl.

In yet another embodiment, the present invention provides a compound of Formula III:

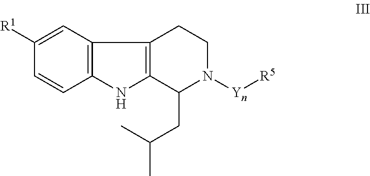

where:

R¹=Cl or Br

Yₙ=bond, CH₂ or C(O);

R⁵=R⁷N(R⁸)R⁹, R⁷C(O)N(R⁸)R⁹, or R⁷SO₂R¹⁰

R⁷=null or optionally substituted alkylene;

R⁸ and R⁹ are independently selected from H, alkyl, alkylene, heteroalkyl, cycloalkyl, heterocycloalkyl or heteroaryl each of which can be optionally substituted or R⁸ can combine with R⁷ and/or R⁹ to form a 4- to 8-membered heterocycle optionally substituted with halogen, alkyl, NR¹⁰, and/or S(O)ₚ; and

R¹⁰=H or optionally substituted alkyl.

It is recognized that compounds of the present invention can contain one or more chiral centers. This invention contemplates all enantiomers, diastereomers, and mixtures of such compounds.

Furthermore, certain embodiments comprise pharmaceutically acceptable salts of compounds according to the present invention. Pharmaceutically acceptable salts comprise, but are not limited to, soluble or dispersible forms of compounds according to the present invention that are suitable for treatment of disease without undue undesirable effects such as allergic reactions or toxicity. Representative pharmaceutically acceptable salts include, but are not limited to, acid addition salts such as acetate, citrate, benzoate, lactate, or phosphate and basic addition salts such as lithium, sodium, potassium, or aluminum.

The term “aryl” as used herein refers to a monocyclic, bicyclic or tricyclic ring system having a total of five to fourteen ring members, wherein at least one ring in the system is aromatic and wherein each ring in the system contains 3 to 7 ring members. The term “aryl” may be used interchangeably with the term “aryl ring”.

The term “heterocycle”, “heterocyclyl”, or “heterocyclic” as used herein means an optionally substituted non-aromatic or aromatic, monocyclic, bicyclic or tricyclic ring systems having three to fourteen ring members in which one or more ring members is a heteroatom, wherein each ring in the system contains 3 to 7 ring members.

The term “heteroaryl” refers to an optionally substituted monocyclic, bicyclic or tricyclic ring systems having three to fourteen ring members wherein at least one ring in the system is aromatic, at least one ring in the system contains one or more heteroatoms, and wherein each ring in the system contains 3 to 7 ring members.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower heteroalkyl, lower heterocycloalkyl, lower haloalkyl, lower haloalkenyl, lower haloalkynyl, lower perhaloalkyl, lower perhaloalkoxy, lower cycloalkyl, aryl, aryloxy, lower alkoxy, lower haloalkoxy, lower acyloxy, CO₂H, lower alkoxycarbonyl, aminocarbonyl, lower alkylcarbonyloxy, lower alkylcarbonyl, lower alkylcarbonylamino, cyano, hydrogen, halogen, hydroxy, amino, lower alkylamino, arylamino, nitro, lower alkylthio, lower haloalkylthio, lower perhaloalkylthio, arylthio, sulfonate, SO₃H, trisubstituted silyl, N₃, SH, pyridinyl, thiophene, furanyl, lower alkylaminocarbonyloxy, lower alkoxycarbonylamino, and lower alkylaminocarbonylamino. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

It is important to recognize that a substituent may be present either singly or multiply when incorporated into the indicated structural unit. For example, the substituent halogen, which means fluorine, chlorine, bromine, or iodine, would indicate that the unit to which it is attached may be substituted with one or more halogen atoms, which may be the same or different.

The compounds of the present invention may be used to treat various microbial infections. They are particularly useful for the treatment of viral infection, and more particularly ocular viral infections such as those caused by adenovirus. Such ocular viral infections include, but are not limited to, viral conjunctivitis, (including adenoviral conjunctivitis), follicular conjunctivitis, ocular keratitis and epidemic keratoconjunctivitis.

The compounds of the present invention are preferably incorporated into topical ophthalmic formulations for delivery to the eye, although other modes of administration known to those of skill in the art are contemplated for ocular and non-ocular uses (e.g., oral, intracameral, topical, intramuscular, etc.) in therapeutically effective amounts (i.e., amounts that eliminate or reduce a patient's viral burden). It is further contemplated that the compounds of the invention may be formulated in intraocular insert or implant devices. The compounds may be combined with ophthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving a compound in a physiologically acceptable isotonic aqueous buffer. Further, the ophthalmic solution may include an ophthalmologically acceptable surfactant to assist in dissolving the compound. Furthermore, the ophthalmic solution may contain an agent to increase viscosity such as hydroxymethylcellulose, hydroxyethylcellulose, hydroxypropyl methylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient may be combined with a preservative in an appropriate vehicle such as mineral oil, liquid lanolin, or white petrolatum. Sterile ophthalmic gel formulations may be prepared by suspending the compound in a hydrophilic base prepared from the combination of, for example, carbopol-974, or the like, according to the published formulations for analogous ophthalmic preparations; preservatives and tonicity agents can be incorporated.

The concentrations of the compounds of the present invention in compositions will vary depending on the intended use of the compositions (e.g., treatment of existing infections or prevention of post-surgical infections), and the relative activity of the compound selected. The compounds will normally be contained in the compositions of the present invention in an amount 0.01 to 5 percent by weight/volume (“w/v %”), but preferably in an amount of 0.1 to 1 w/v %.

In certain embodiments, a composition of the invention has a pH of about 3.0 to about 8.5. In one embodiment, an ophthalmic composition of the present invention has a pH of 3.5-8.0, preferably a pH of 4.0-7.5, and most preferably a pH of 4.5-6.5.

The formulations of the present invention that are adapted for topical administration to the eye are preferably isotonic, or slightly hypotonic in order to combat any hypertonicity of tears caused by evaporation and/or disease. This may require a tonicity agent to bring the osmolality of the formulation to a level at or near 210-320 milliosmoles per kilogram (mOsm/kg). The formulations of the present invention generally have an osmolality in the range of 220-320 mOsm/kg, and preferably have an osmolality in the range of 235-300 mOsm/kg. The ophthalmic formulations will generally be formulated as sterile aqueous solutions.

The compounds of the present invention can also be used in combination with other antiinfective or anti-inflammatory agents. Preferred combinations include compounds of the present invention with steroidal or non-steroidal anti-inflammatory compounds such as dexamethasone, nepafenac, dexamethasone, loteprednol, rimexolone, prednisolone, fluorometholone, and hydrocortisone.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

Additional compounds of the present invention (Compounds 1-110) include the structures shown in TABLE 1.

TABLE 1

| Compound # | Structure | Spectral Data |
|---|---|---|
| 1 | 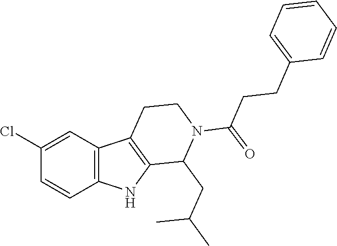 | ¹H NMR (400 MHz, CD₄OD) δ 7.32 (d, J = 2.02 Hz, 1H), 7.21-7.26 (m, 5H), 7.14-7.18 (m, 1H), 6.99-7.03 (m, 1H), 5.81-5.87 (m, 1H), 4.07-4.14 (m, 1H), 3.39-3.49 (m, 1H), 2.58-3.00 (m, 6H), 1.75-1.82 (m, 1H), 1.58-1.64 (m, 2H), 1.09 (d, J = 5.81 Hz, 3H), 0.93 (d, J = 6.32 Hz, 3H); LCMS (+ESI) 263 (M⁺). |
| 2 | 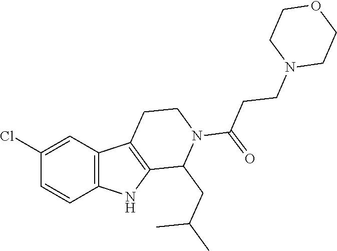 [SMILES: CC(C)CC1c2[nH]c3ccc(Cl)cc3c2CCN1C(=O)CCN1CCOCC1] | ¹H NMR (400 MHz, CD₃OD) δ 7.36 (d, J = 1.77 Hz, 1H), 7.24 (d, J = 8.59 Hz, 1H), 7.01 (dd, J = 2.02, 8.59 Hz, 1H), 5.81-5.87 (m, 1H), 4.14-4.21 (m, 1H), 3.67-3.71 (m, 3H), 3.50-3.60 (m, 2H), 2.65-2.84 (m, 6H), 2.51-2.55 (m, 4H), 1.80-1.87 (m, 1H), 1.63-1.73 (m, 2H), 1.10 (d, J = 6.32 Hz, 3H), 0.96 (d, J = 6.32 Hz, 3H); LCMS (+ESI) 404 (M⁺). |
| 3 | 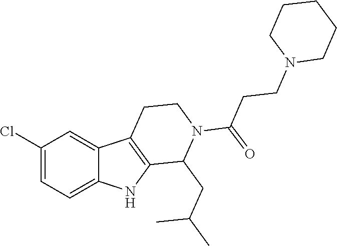 [SMILES: CC(C)CC1c2[nH]c3ccc(Cl)cc3c2CCN1C(=O)CCN1CCCCC1] | ¹H NMR (400 MHz, CD₃OD) δ 7.36 (d, J = 2.02 Hz, 1H), 7.24 (d, J = 8.84 Hz, 1H), 6.99-7.03 (m, 1H), 5.80-5.86 (m, 1H), 4.13-4.20 (m, 1H), 3.50-3.60 (m, 1H), 2.72 (d, J = 6.06 Hz, 6H), 2.49-2.55 (m, 4H), 1.78-1.87 (m, 1H), 1.60-1.70 (m, 6H), 1.46-1.54 (m, 2H), 1.10 (d, J = 6.06 Hz, 3H), 0.96 (d, J = 6.57 Hz, 3H); LCMS (+ESI) 402 (M⁺). |
| 4 | 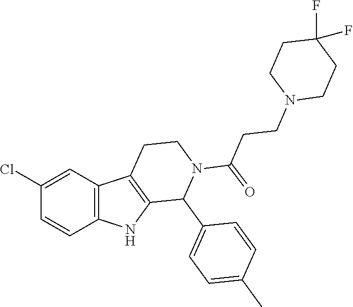 [SMILES: Cc1ccc(C2c3[nH]c4ccc(Cl)cc4c3CCN2C(=O)CCN2CCC(F)(F)CC2)cc1] | ¹H NMR (400 MHz, CD₃OD) δ 7.46 (d, J = 2.02 Hz, 1H), 7.24 (d, J = 8.59 Hz, 1H), 7.13 (d, J = 2.02 Hz, 3H), 7.06 (dd, J = 2.02, 8.59 Hz, 1H), 6.93 (s, 1H), 4.75-4.85 (m, 1H), 4.10 (d, J = 7.07 Hz, 1H), 3.37-3.47 (m, 1H), 2.57-2.98 (m, 10H), 2.30 (s, 3H), 1.92 (br. s., 4H); LCMS (+ESI) 472 (M⁺). |
| 5 | 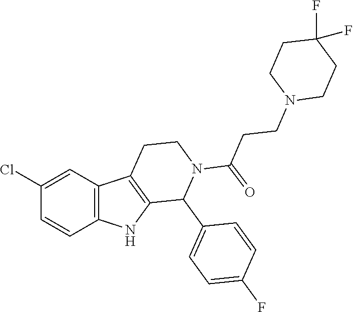 [SMILES: O=C(CCN1CCC(F)(F)CC1)N1CCc2c([nH]c3ccc(Cl)cc23)C1c1ccc(F)cc1] | ¹H NMR (600 MHz, CD₃OD) δ 7.47 (d, J = 1.79 Hz, 1H), 7.29 (s, 2H), 7.25 (d, J = 8.56 Hz, 1H), 7.04 (s, 3H), 6.94 (s, 1H), 4.11 (s, 1H), 3.37-3.43 (m, 1H), 2.85-2.98 (m, 2H), 2.74-2.82 (m, 4H), 2.61 (br. s., 4H), 1.93 (br. s., 4H); LCMS (+ESI) 476 (M⁺). |
| 6 | 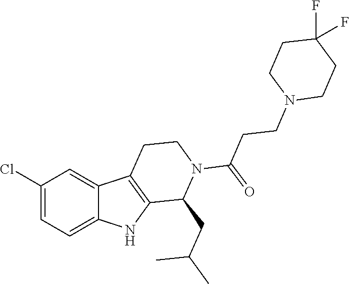 [SMILES: CC(C)C[C@H]1c2[nH]c3ccc(Cl)cc3c2CCN1C(=O)CCN1CCC(F)(F)CC1] | ¹H NMR (400 MHz, DMSO-d₆) δ 11.02-11.16 (m, 1H), 7.41 (d, J = 2.1 Hz, 1H), 7.30 (dd, J = 0.3, 8.5 Hz, 1H), 7.03 (dd, J = 2.1, 8.5 Hz, 1H), 5.65-5.80 (m, 1H), 4.03-4.19 (m, 1H), 3.34-3.47 (m, 1H), 2.61-2.79 (m, 5H), 2.53 (s, 5H), 1.85-1.99 (m, 4H), 1.70-1.80 (m, 1H), 1.53-1.67 (m, 2H), 1.03 (d, J = 6.0 Hz, 3H), 0.90 (d, J = 6.3 Hz, 3H); LCMS (+ESI) 437 (M⁺). |
| 7 | 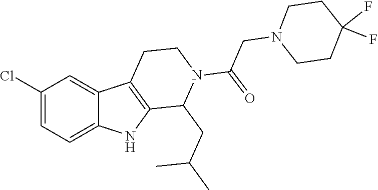 [SMILES: CC(C)CC1c2[nH]c3ccc(Cl)cc3c2CCN1C(=O)CN1CCC(F)(F)CC1] | ¹H NMR (600 MHz, CD₃OD) δ 7.37 (d, J = 1.98 Hz, 1H), 7.24 (d, J = 8.56 Hz, 1H), 7.02 (dd, J = 1.98, 8.56 Hz, 1H), 5.78-5.82 (m, 1H), 4.24-4.30 (m, 1H), 3.48-3.54 (m, 1H), 3.42 (s, 2H), 2.85-2.92 (m, 1H), 2.72-2.77 (m, 1H), 2.61-2.70 (m, 3H), 1.97-2.05 (m, 4H), 1.80-1.89 (m, 2H), 1.68 (s, 2H), 1.11 (d, J = 6.02 Hz, 3H), 0.96 (d, J = 6.31 Hz, 3H); LCMS (+ESI) 424 (M⁺). |
| 8 | 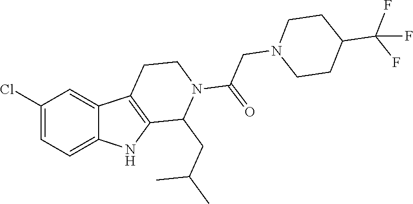 [SMILES: CC(C)CC1c2[nH]c3ccc(Cl)cc3c2CCN1C(=O)CN1CCC(C(F)(F)F)CC1] | ¹H NMR (400 MHz, CD₃OD) δ 7.36 (d, J = 1.77 Hz, 1H), 7.24 (d, J = 8.59 Hz, 1H), 7.00-7.03 (m, 1H), 5.77-5.82 (m, 1H), 4.25-4.34 (m, 1H), 3.45-3.55 (m, 1H), 3.36 (d, J = 0.76 Hz, 2H), 2.82-3.16 (m, 3H), 2.71-2.78 (m, 1H), 2.08-2.18 (m, 3H), 1.79-1.90 (m, 3H), 1.57-1.71 (m, 4H), 1.11 (d, J = 6.06 Hz, 3H), 0.96 (d, J = 6.32 Hz, 3H); LCMS (+ESI) 456 (M⁺). |
| 9 | 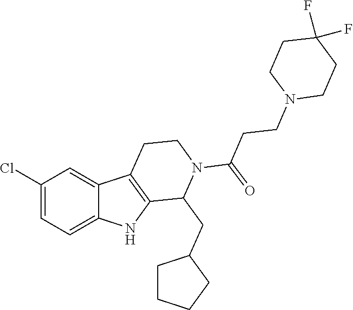 [SMILES: O=C(CCN1CCC(F)(F)CC1)N1CCc2c([nH]c3ccc(Cl)cc23)C1CC1CCCC1] | ¹H NMR (400 MHz, CDCl₃) δ 8.02 (s, 1H), 7.39 (d, J = 2.0 Hz, 1H), 7.25 (s, 1H), 7.06 (dd, J = 2.0, 8.5 Hz, 1H), 5.81 (dd, J = 5.6, 9.2 Hz, 1H), 4.03-4.06 (m, 1H), 3.43-3.52 (ddd, J = 4.5, 11.7, 14.3 Hz, 1H), 2.54-2.85 (m, 12H), 1.56-1.97 (m, 10H), 1.52 (m, 1H), 1.16 (m, 2H); LCMS (+ESI) 464 (M⁺). |
| 10 | 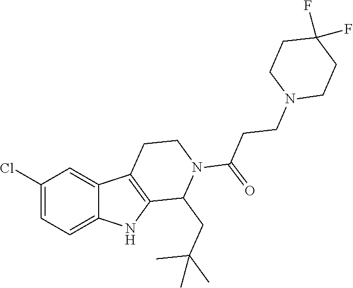 [SMILES: CC(C)(C)CC1c2[nH]c3ccc(Cl)cc3c2CCN1C(=O)CCN1CCC(F)(F)CC1] | ¹H NMR (400 MHz, CDCl₃) δ 8.02 (s, 1H), 7.39 (d, J = 2.0 Hz, 1H), 7.25 (s, 1H), 7.06 (dd, J = 2.0, 8.5 Hz, 1H), 5.95 (dd, J = 5.6, 9.2 Hz, 1H), 4.06 (dd, J = 5.0, 14.3 Hz, 1H), 3.51-3.62 (ddd, J = 4.5, 11.7, 14.3 Hz, 1H), 2.51-2.85 (m, 10H), 1.54-2.08 (m, 5H), 1.52 (m, 1H), 1.01 (s, 9H); LCMS (+ESI) 452 (M⁺). |
| 11 | 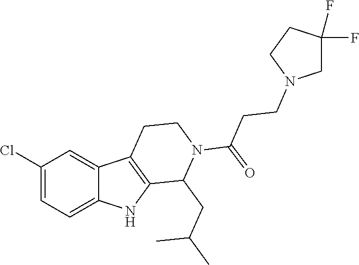 [SMILES: CC(C)CC1c2[nH]c3ccc(Cl)cc3c2CCN1C(=O)CCN1CCC(F)(F)C1] | ¹H NMR (400 MHz, CD₃OD) δ 7.35-7.37 (m, 1H), 7.22-7.26 (m, 1H), 7.00-7.03 (m, 1H), 5.81-5.88 (m, 1H), 4.14-4.21 (m, 1H), 3.50-3.60 (m, 1H), 2.92-3.01 (m, 2H), 2.83 (m, 8H), 2.19-2.32 (m, 2H), 1.79-1.87 (m, 1H), 1.61-1.73 (m, 2H), 1.10 (d, J = 6.06 Hz, 3H), 0.96 (d, J = 6.32 Hz, 3H); LCMS (+ESI) 424 (M⁺). |
| 12 | 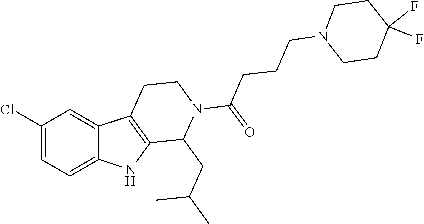 [SMILES: CC(C)CC1c2[nH]c3ccc(Cl)cc3c2CCN1C(=O)CCCN1CCC(F)(F)CC1] | ¹H NMR (400 MHz, CD₃OD) δ 7.36 (d, J = 1.77 Hz, 1H), 7.24 (d, J = 8.59 Hz, 1H), 7.01 (dd, J = 2.02, 8.59 Hz, 1H), 5.81-5.89 (m, 1H), 4.13-4.22 (m, 1H), 3.48-3.65 (m, 2H), 2.39-2.88 (m, 9H), 1.79-2.06 (m, 7H), 1.67 (s, 2H), 1.08-1.14 (m, 3H), 0.96 (d, J = 6.32 Hz, 3H); LCMS (+ESI) 453 (M⁺). |
| 13 | 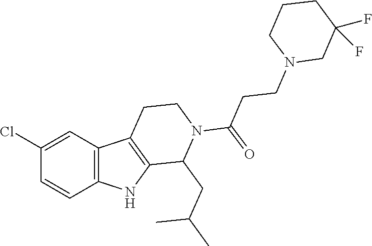 [SMILES: CC(C)CC1c2[nH]c3ccc(Cl)cc3c2CCN1C(=O)CCN1CCCC(F)(F)C1] | ¹H NMR (400 MHz, CD₃OD) δ 7.34-7.38 (m, 1H), 7.21-7.27 (m, 1H), 6.99-7.03 (m, 1H), 5.81-5.86 (m, 1H), 4.15-4.24 (m, 1H), 3.49-3.60 (m, 1H), 2.50-2.89 (m, 10H), 1.58-1.97 (m, 7H), 1.10 (d, J = 6.06 Hz, 3H), 0.96 (d, J = 6.57 Hz, 3H); LCMS (+ESI) 438 (M⁺). |
| 14 | 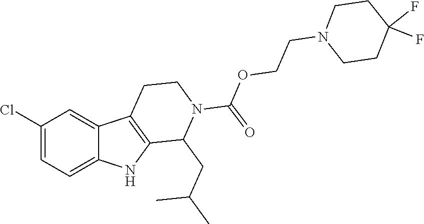 [SMILES: CC(C)CC1c2[nH]c3ccc(Cl)cc3c2CCN1C(=O)OCCN1CCC(F)(F)CC1] | ¹H NMR (400 MHz, CDCl₃) δ 8.02 (s, 1H), 7.40 (d, J = 2.0 Hz, 1H), 7.21 (d, J = 8.0 Hz, 1H), 7.10 (dd, J = 2.0, 8.5 Hz, 1H), 5.41 (m, 1H), 4.11-4.47 (m, 3H), 3.17-3.20 (m, 1H), 2.58-2.73 (m, 8H), 1.89-2.04 (m, 4H), 1.66-1.80 (m, 2H), 1.53-1.55 (m, 1H), 1.05 (d, J = 6.4 Hz, 3H), 0.95 (d, J = 6.5 Hz, 3H); LCMS (+ESI) 454 (M⁺). |
| 15 | 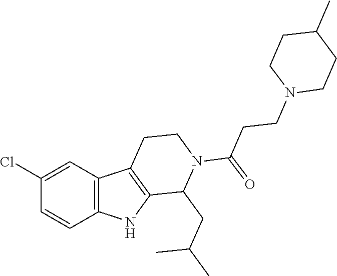 [SMILES: CC(C)CC1c2[nH]c3ccc(Cl)cc3c2CCN1C(=O)CCN1CCC(C)CC1] | ¹H NMR (400 MHz, CD₃OD) δ 7.36 (d, J = 2.02 Hz, 1H), 7.24 (dd, J = 0.51, 8.59 Hz, 1H), 6.99-7.03 (m, 1H), 5.80-5.86 (m, 1H), 4.29-4.32 (m, 1H), 3.50-3.60 (m, 1H), 2.92-3.01 (m, 2H), 2.73 (s, 7H), 2.06-2.14 (m, 2H), 1.63-1.80 (m, 5H), 1.35-1.45 (m, 1H), 1.10 (d, J = 6.06 Hz, 3H), 0.95 (t, J = 6.44 Hz, 7H); LCMS (+ESI) 416 (M⁺). |
| 16 | 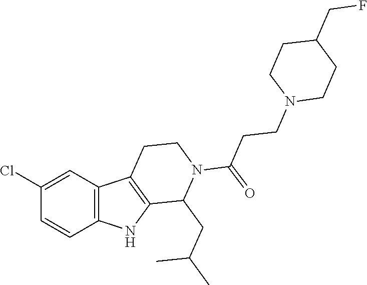 [SMILES: CC(C)CC1c2[nH]c3ccc(Cl)cc3c2CCN1C(=O)CCN1CCC(CF)CC1] | ¹H NMR (600 MHz, CD₃OD) δ 7.37 (d, J = 1.98 Hz, 1H), 7.24 (d, J = 8.56 Hz, 1H), 7.00-7.03 (m, 1H), 5.82-5.85 (m, 1H), 4.21-4.31 (m, 2H), 4.15-4.20 (m, 1H), 3.52-3.59 (m, 1H), 3.00-3.06 (m, 2H), 2.66-2.87 (m, 7H), 2.09-2.15 (m, 2H), 1.81-1.87 (m, 1H), 1.62-1.77 (m, 4H), 1.33-1.42 (m, 2H), 1.10 (d, J = 6.12 Hz, 3H), 0.96 (d, J = 6.40 Hz, 3H); LCMS (+ESI) 434 (M⁺). |
| 17 | 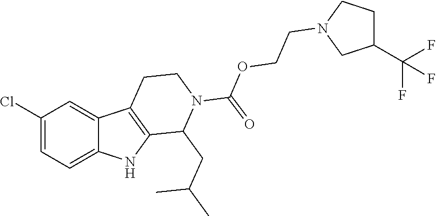 [SMILES: CC(C)CC1c2[nH]c3ccc(Cl)cc3c2CCN1C(=O)OCCN1CCC(C(F)(F)F)C1] | ¹H NMR (400 MHz, CDCl₃) δ 8.02 (s, 1H), 7.38 (d, J = 2.0 Hz, 1H), 7.18 (d, J = 8.0 Hz 1H), 7.09 (dd, J = 2.0, 8.5 Hz, 1H), 5.21-5.45 (m, 1H), 4.21-4.53 (m, 3H), 3.11-3.25 (m, 1H), 2.50-2.98 (m, 8H), 1.85-2.04 (m, 1H), 1.66-1.80 (m, 2H), 1.53-1.55 (m, 1H), 1.05 (d, J = 6.4 Hz, 3H), 0.95 (d, J = 6.5 Hz, 3H); LCMS (+ESI) 472 (M⁺). |
| 18 | 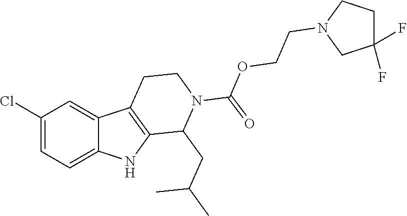 [SMILES: CC(C)CC1c2[nH]c3ccc(Cl)cc3c2CCN1C(=O)OCCN1CCC(F)(F)C1] | ¹H NMR (600 MHz, CD₃OD) δ 7.35 (s, 1H), 7.21 (d, J = 8.0 Hz, 1H), 7.0 (dd, J = 2.0, 8.5 Hz, 1H), 5.41 (m, 1H), 4.11-4.47 (m, 3H), 3.12-3.31 (m, 1H), 2.57-2.98 (m, 8H), 2.12-2.28 (m, 2H), 1.71-1.85 (m, 2H), 1.61-1.68 (m, 1H), 1.11 (d, J = 6.4 Hz, 3H), 0.96 (d, J = 6.5 Hz, 3H); LCMS (+ESI) 440 (M⁺). |
| 19 | 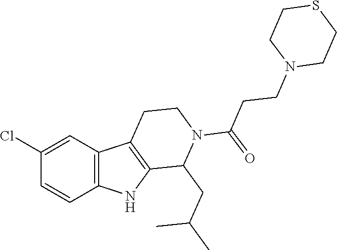 [SMILES: CC(C)CC1c2[nH]c3ccc(Cl)cc3c2CCN1C(=O)CCN1CCSCC1] | ¹H NMR (400 MHz, CD₃OD) δ 7.36 (d, J = 1.77 Hz, 1H), 7.24 (dd, J = 0.51, 8.59 Hz, 1H), 7.02 (d, J = 2.02 Hz, 1H), 5.80-5.86 (m, 1H), 4.13-4.21 (m, 1H), 3.49-3.60 (m, 1H), 2.63-2.84 (m, 13H), 2.47-2.51 (m, 1H), 1.61-1.88 (m, 3H), 1.10 (d, J = 6.06 Hz, 3H), 0.96 (d, J = 6.57 Hz, 3H); LCMS (+ESI) 420 (M⁺). |
| 20 | 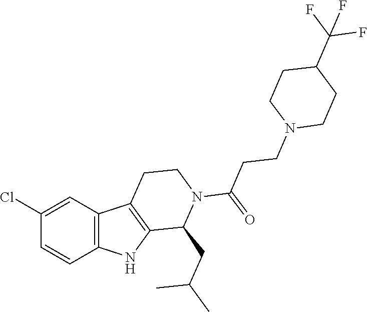 [SMILES: CC(C)C[C@H]1c2[nH]c3ccc(Cl)cc3c2CCN1C(=O)CCN1CCC(C(F)(F)F)CC1] | ¹H NMR (400 MHz, CD₂CL₂) δ 8.02 (s, 1H), 7.45 (d, J = 2.0 Hz, 1H), 7.29 (dd, J = 0.4, 8.6 Hz, 1H), 7.12 (dd, J = 2.1, 8.6 Hz, 1H), 5.89 (dd, J = 4.4, 10.0 Hz, 1H), 4.09 (dd, J = 4.8, 14.3 Hz, 1H), 3.51 (ddd, J = 4.6, 11.8, 14.3 Hz, 1H), 3.04 (t, J = 10.0 Hz, 2H), 2.52-2.90 (m, 6H), 2.04 (t, J = 11.4 Hz, 2H), 1.68-1.92 (m, 4H), 1.59 (s, 4H), 1.10 (d, J = 6.4 Hz, 3H), 1.01 (d, J = 6.6 Hz, 3H); LCMS (+ESI) 470 (M⁺). |
| 21 | 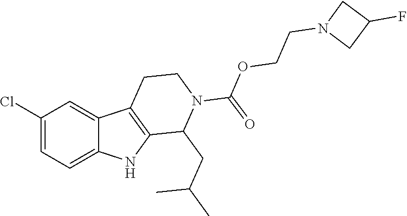 [SMILES: CC(C)CC1c2[nH]c3ccc(Cl)cc3c2CCN1C(=O)OCCN1CC(F)C1] | ¹H NMR (400 MHz, CD₃OD) δ 7.35 (d, J = 2.0 Hz, 1H), 7.14 (d, J = 8.0 Hz, 1H), 6.91 (dd, J = 2.0, 8.5 Hz, 1H), 5.21-5.39 (m, 1H), 4.85-5.18 (m, 1H), 4.21-4.35 (m, 1H), 3.49-3.70 (m, 2H), 3.12-3.31 (m, 3H), 2.71-2.75 (m, 3H), 2.57 (m, 1H), 1.61-1.85 (m, 2H), 1.54-1.57 (m, 1H), 1.03 (d, J = 6.4 Hz, 3H), 0.88 (d, J = 6.5 Hz, 3H); LCMS (+ESI) 408 (M⁺). |
| 22 | 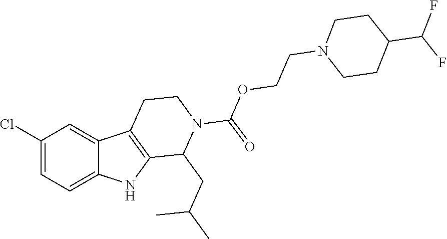 [SMILES: CC(C)CC1c2[nH]c3ccc(Cl)cc3c2CCN1C(=O)OCCN1CCC(C(F)F)CC1] | ¹H NMR (400 MHz, CD₃OD) δ 7.25 (d, J = 2.0 Hz, 1H), 7.12 (d, J = 8.0 Hz, 1H), 6.92 (dd, J = 2.0, 8.5 Hz, 1H), 5.21-5.65 (m, 2H), 4.18-4.22 (m, 3H), 3.12-3.31 (m, 1H), 2.71-2.98 (m, 2H), 2.52-2.68 (m, 4H), 1.70-1.74 (m, 2H), 1.50-1.64 (m, 6H), 1.15-1.45 (m, 2H), 1.07 (d, J = 6.4 Hz, 3H), 0.88 (d, J = 6.5 Hz, 3H); LCMS (+ESI) 468 (M⁺). |
| 23 | 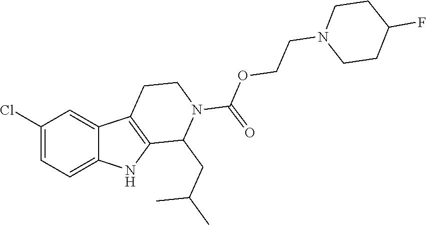 [SMILES: CC(C)CC1c2[nH]c3ccc(Cl)cc3c2CCN1C(=O)OCCN1CCC(F)CC1] | ¹H NMR (400 MHz, CD₃OD) δ 7.35 (d, J = 2.0 Hz, 1H), 7.23 (d, J = 8.0 Hz, 1H), 7.0 (dd, J = 2.0, 8.5 Hz, 1H), 5.35-5.38 (m, 1H), 4.53-4.71 (m, 1H), 4.25-4.35 (m, 3H), 3.21-3.35 (m, 1H), 2.55-2.85 (m, 6H), 2.38-2.55 (m, 2H), 1.70-1.83 (m, 6H), 1.51-1.63 (m, 1H), 1.11 (d, J = 6.4 Hz, 3H), 0.97 (d, J = 6.5 Hz, 3H); LCMS (+ESI) 436 (M⁺). |
| 24 | 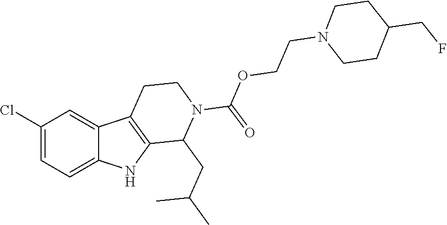 [SMILES: CC(C)CC1c2[nH]c3ccc(Cl)cc3c2CCN1C(=O)OCCN1CCC(CF)CC1] | ¹H NMR (400 MHz, CD₃OD) δ 7.26 (s, 1H), 7.13 (d, J = 8.0 Hz, 1H), 6.93 (dd, J = 2.0, 8.5 Hz, 1H), 5.28-5.31 (m, 1H), 4.04-4.73 (m, 5H), 3.17-3.23 (m, 1H), 2.83-2.89 (m, 2H), 2.55-2.68 (m, 4H), 1.99-1.22 (m, 2H), 1.52-1.85 (m, 7H), 1.50-1.64 (m, 6H), 1.21-1.35 (m, 1H), 1.03 (d, J = 6.4 Hz, 3H), 0.89 (d, J = 6.5 Hz, 3H); LCMS (+ESI) 450 (M⁺). |
| 25 | 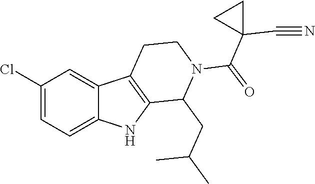 [SMILES: CC(C)CC1c2[nH]c3ccc(Cl)cc3c2CCN1C(=O)C1(C#N)CC1] | ¹H NMR (400 MHz, CD₃OD) δ 7.39 (d, J = 2.02 Hz, 1H), 7.24 (s, 1H), 6.99-7.07 (m, 1H), 5.66-5.74 (m, 1H), 4.63-4.71 (m, 1H), 3.69-3.79 (m, 1H), 2.98-3.08 (m, 1H), 2.79-2.87 (m, 1H), 1.83-1.93 (m, 1H), 1.52-1.75 (m, 6H), 1.08 (d, J = 6.06 Hz, 3H), 0.98 (d, J = 6.32 Hz, 3H); LCMS (+ESI) 356 (M⁺). |
| 26 | 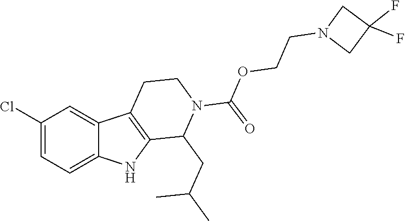 [SMILES: CC(C)CC1c2[nH]c3ccc(Cl)cc3c2CCN1C(=O)OCCN1CC(F)(F)C1] | ¹H NMR (600 MHz, CD₃OD) δ 7.26 (s, 1H), 7.18 (d, J = 8.0 Hz, 1H), 6.91 (dd, J = 2.0, 8.5 Hz, 1H), 5.22-5.38 (m, 1H), 4.01-4.33 (m, 3H), 3.51-3.62 (m, 5H), 2.52-2.85 (m, 4H), 1.71-1.85 (m, 2H), 1.51-1.59 (m, 1H), 1.01 (d, J = 6.4 Hz, 3H), 0.87 (d, J = 6.5 Hz, 3H); LCMS (+ESI) 426 (M⁺). |
| 27 | 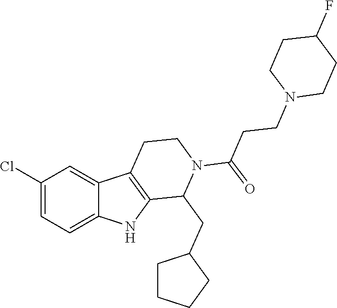 [SMILES: O=C(CCN1CCC(F)CC1)N1CCc2c([nH]c3ccc(Cl)cc23)C1CC1CCCC1] | ¹H NMR (600 MHz, CD₃OD) δ 7.36 (s, 1H), 7.23 (d, J = 4.0 Hz, 1H), 7.02 (dd, J = 2.0, 8.5 Hz, 1H), 5.77 (dd, J = 5.6, 9.1 Hz, 1H), 4.50-4.75 (m, 1H), 4.15-4.21 (dd, J = 5.0, 14.3 Hz, 1H), 3.51-3.61 (ddd, J = 4.5, 11.7, 14.3 Hz, 1H), 2.31-2.82 (m, 10H), 1.51-1.95 (m, 13H), 1.65-1.85 (m, 2H); LCMS (+ESI) 446 (M⁺). |
| 28 | 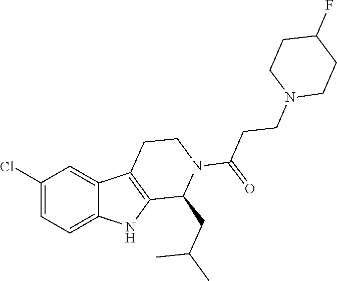 [SMILES: CC(C)C[C@H]1c2[nH]c3ccc(Cl)cc3c2CCN1C(=O)CCN1CCC(F)CC1] | ¹H NMR (400 MHz, CD₂Cl₂) δ 8.02 (s, 1H), 7.38 (d, J = 2.1 Hz, 1H), 7.23 (dd, J = 0.4, 8.6 Hz, 1H), 7.06 (dd, J = 2.0, 8.6 Hz, 1H), 5.84 (dd, J = 4.4, 9.9 Hz, 1H), 4.51-4.72 (m, 1H), 3.99-4.08 (m, 1H), 3.45 (ddd, J = 4.6, 11.7, 14.3 Hz, 1H), 2.48-2.83 (m, 7H), 2.37 (d, J = 3.6 Hz, 2H), 1.49-1.95 (m, 8H), 1.05 (d, J = 6.4 Hz, 3H), 0.95 (d, J = 6.6 Hz, 3H); LCMS (+ESI) 420 (M⁺). |
| 29 | 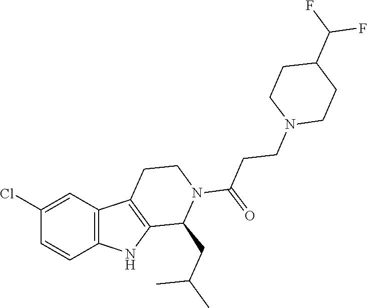 [SMILES: CC(C)C[C@H]1c2[nH]c3ccc(Cl)cc3c2CCN1C(=O)CCN1CCC(C(F)F)CC1] | ¹H NMR (600 MHz, CD₂Cl₂-d₂) δ 8.00 (br. s., 1H), 7.41 (d, J = 1.9 Hz, 1H), 7.25 (d, J = 8.7 Hz, 1H), 7.08 (dd, J = 1.98, 8.6 Hz, 1H), 5.86 (dd, J = 4.4, 10.1 Hz, 1H), 5.47-5.69 (m, 1H), 4.06 (dd, J = 5.1, 14.3 Hz, 1H), 3.47 (ddd, J = 4.3, 12.1, 14.3 Hz, 1H), 2.97 (t, J = 12.1 Hz, 2H), 2.45-2.84 (m, 6H), 1.97-2.04 (m, 2H), 1.64-1.83 (m, 5H), 1.52-1.60 (m, 1H), 1.38-1.48 (m, 2H), 1.07 (d, J = 6.5 Hz, 3H), 0.97 (d, J = 6.7 Hz, 3H); LCMS (+APCI) 452 (M⁺). |
| 30 | 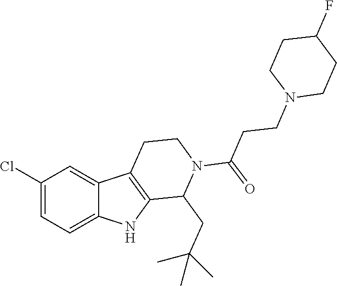 [SMILES: CC(C)(C)CC1c2[nH]c3ccc(Cl)cc3c2CCN1C(=O)CCN1CCC(F)CC1] | ¹H NMR (400 MHz, CD₂Cl₂) δ 7.99 (s, 1H), 7.39 (d, J = 2.1 Hz, 1H), 7.23 (d, J = 4.0 Hz, 1H), 7.07 (dd, J = 2.0, 8.5 Hz, 1H), 5.96 (dd, J = 5.6, 9.1 Hz, 1H), 4.55-4.69 (m, 1H), 4.02 (dd, J = 5.0, 14.3 Hz, 1H), 3.56 (ddd, J = 4.5, 11.7, 14.3 Hz, 1H), 2.51-2.81 (m, 8H), 2.36-2.38 (m, 2H), 1.79-1.89 (m, 4H), 1.58-1.62 (m, 2H), 1.01 (s, 9H); LCMS (+ESI) 434 (M⁺). |
| 31 | 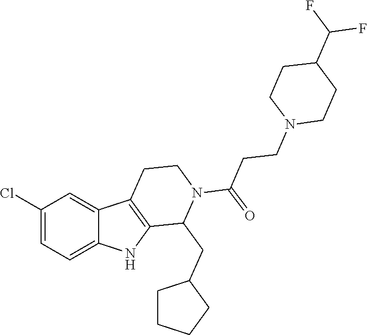 [SMILES: O=C(CCN1CCC(C(F)F)CC1)N1CCc2c([nH]c3ccc(Cl)cc23)C1CC1CCCC1] | ¹H NMR (400 MHz, CD₂Cl₂) δ 8.17 (s, 1H), 7.32 (s, 1H), 7.15 (d, J = 4.0 Hz, 1H), 6.98 (dd, J = 2.0, 8.5 Hz, 1H), 5.71-5.75 (m, 1H), 5.35-5.68 (m, 1H), 4.01 (dd, J = 5.0, 14.3 Hz, 1H), 3.40 (ddd, J = 4.5, 11.7, 14.3 Hz, 1H), 2.48-2.91 (m, 8H), 1.05-2.03 (m, 18H); LCMS (+ESI) 478 (M⁺). |
| 32 | 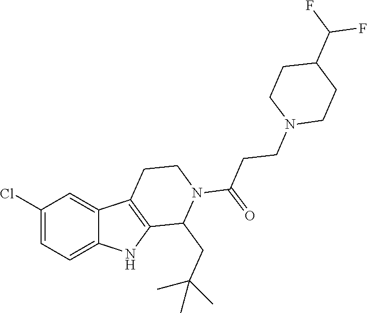 [SMILES: CC(C)(C)CC1c2[nH]c3ccc(Cl)cc3c2CCN1C(=O)CCN1CCC(C(F)F)CC1] | ¹H NMR (400 MHz, CD₂Cl₂) δ 8.06 (s, 1H), 7.30 (d, J = 2.1 Hz, 1H), 7.15 (d, J = 4.0 Hz, 1H), 6.99 (dd, J = 2.0, 8.5 Hz, 1H), 5.88 (dd, J = 5.6, 9.1 Hz, 1H), 5.32-5.64 (m, 1H), 3.94 (dd, J = 5.0, 14.3 Hz, 1H), 3.47 (ddd, J = 4.5, 11.7, 14.3 Hz, 1H), 2.86-2.89 (m, 2H), 2.43-2.72 (m, 6H), 1.88-2.03 (m, 2H), 1.76-1.82 (m, 2H), 1.61-1.66 (m, 2H), 1.50-1.53 (m, 1H), 1.32-1.36 (m, 2H), 0.9 (s, 9H); LCMS (+ESI) 466 (M⁺). |
| 33 | 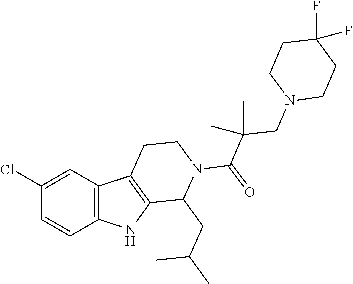 [SMILES: CC(C)CC1c2[nH]c3ccc(Cl)cc3c2CCN1C(=O)C(C)(C)CN1CCC(F)(F)CC1] | LCMS (+APCI) 466 (M⁺) |
| 34 | 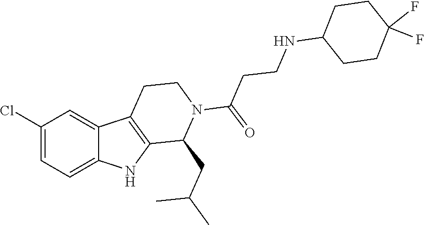 [SMILES: CC(C)C[C@H]1c2[nH]c3ccc(Cl)cc3c2CCN1C(=O)CCNC1CCC(F)(F)CC1] | ¹H NMR (400 MHz, CD₂Cl₂) δ 8.3 (s, 1H), 7.29 (s, 1H), 7.18 (d, J = 8.0 Hz, 1H), 6.99 (dd, J = 2.0, 8.5 Hz, 1H), 5.72 (dd, J = 4.0, 8.2 Hz, 1H), 3.82 (m, 1H), 3.35 (m, 1H), 2.91-2.98 (m, 2H), 2.51-2.75 (m, 3H), 1.92-2.12 (m, 4H), 1.48-1.76 (m, 7H), 1.18 (d, J = 6.4 Hz, 3H), 0.88 (d, J = 6.5 Hz, 3H); LCMS (+ESI) 452 (M⁺). |
| 35 | 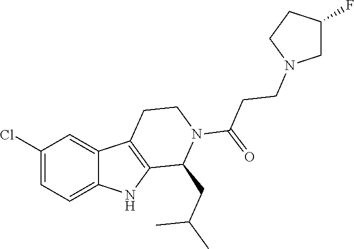 [SMILES: CC(C)C[C@H]1c2[nH]c3ccc(Cl)cc3c2CCN1C(=O)CCN1CC[C@H](F)C1] | ¹H NMR (400 MHz, CD₃OD) δ 7.26 (s, 1H), 7.13 (dd, J = 0.4, 8.6 Hz, 1H), 6.92 (dd, J = 2.0, 8.6 Hz, 1H), 5.74 (dd, J = 4.3, 10.0 Hz, 1H), 5.02-5.22 (m, 1H), 4.06 (dd, J = 4.8, 14.2 Hz, 1H), 3.21 (ddd, J = 4.5, 11.7, 14.2 Hz, 1H), 2.88 (m, 2H), 2.58-2.76 (m, 7H), 2.36 (m, 1H), 2.05 (m, 1H), 1.73 (m, 1H), 1.71 (m, 1H), 1.57 (m, 2H), 1.02 (d, J = 6.40 Hz, 3H), 0.86 (d, J = 6.5 Hz, 3H); LCMS (+ESI) 406 (M⁺). |
| 36 | 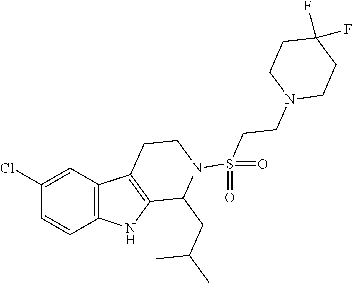 [SMILES: CC(C)CC1c2[nH]c3ccc(Cl)cc3c2CCN1S(=O)(=O)CCN1CCC(F)(F)CC1] | ¹H NMR (600 MHz, CD₂Cl₂) δ 7.96 (br. s., 1H), 7.45 (d, J = 1.98 Hz, 1H), 7.29 (d, J = 8.56 Hz, 1H), 7.13 (dd, J = 2.07, 8.56 Hz, 1H), 4.98 (dd, J = 4.19, 10.02 Hz, 1H), 4.07-4.12 (m, 1H), 3.47 (ddd, J = 4.80, 12.14, 14.96 Hz, 1H), 2.96-3.10 (m, 2H), 2.89-2.96 (m, 1H), 2.81 (ddd, J = 5.60, 9.62, 12.92 Hz, 1H), 2.65-2.75 (m, 2H), 2.40 (t, J = 5.60 Hz, 4H), 1.8-1.96 (m, 6H), 1.53-1.58 (m, 1H), 1.06 (d, J = 6.49 Hz, 3H), 0.98-1.02 (m, 3H); LCMS (+ESI) 474 (M⁺). |
| 37 | 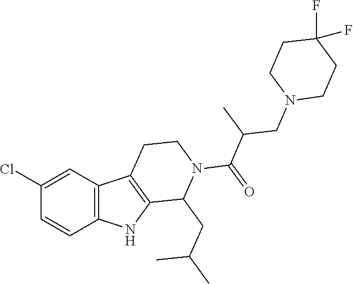 [SMILES: CC(C)CC1c2[nH]c3ccc(Cl)cc3c2CCN1C(=O)C(C)CN1CCC(F)(F)CC1] | LCMS (+APCI) 452 (M⁺) |
| 38 | 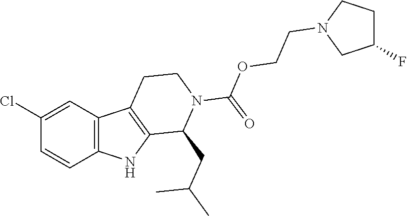 [SMILES: CC(C)C[C@H]1c2[nH]c3ccc(Cl)cc3c2CCN1C(=O)OCCN1CC[C@H](F)C1] | ¹H NMR (400 MHz, CD₃OD) δ 7.25 (s, 1H), 7.15 (d, J = 8.0 Hz, 1H), 6.92 (dd, J = 2.0, 8.5 Hz, 1H), 5.22-5.38 (m, 1H), 4.91-5.28 (m, 1H), 4.11-4.32 (m, 3H), 3.21-3.35 (m, 1H), 2.56-2.75 (m, 7H), 2.38-2.49 (m, 1H), 1.62-2.22 (m, 4H), 1.51-1.63 (m, 1H), 1.02 (d, J = 6.4 Hz, 3H), 0.86 (d, J = 6.5 Hz, 3H); LCMS (+ESI) 422 (M⁺). |
| 39 | 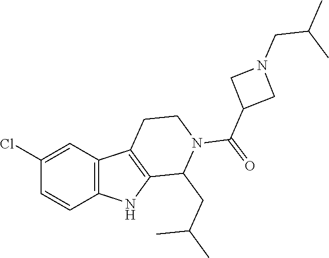 [SMILES: CC(C)CC1c2[nH]c3ccc(Cl)cc3c2CCN1C(=O)C1CN(CC(C)C)C1] | LCMS (+APCI) 402 (M⁺) |
| 40 | 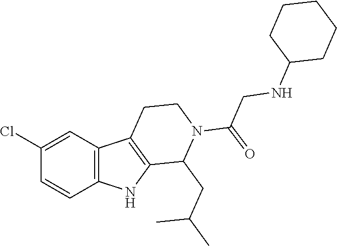 [SMILES: CC(C)CC1c2[nH]c3ccc(Cl)cc3c2CCN1C(=O)CNC1CCCCC1] | ¹H NMR (600 MHz, CD₂Cl₂) δ 7.37 (s, 1H), 7.24 (d, J = 8.56 Hz, 1H), 7.04 (d, J = 7.15 Hz, 1H), 5.73-5.76 (m, 1H), 3.93-4.16 (m, 4H), 3.86 (dd, J = 5.08, 14.21 Hz, 1H), 3.52-3.59 (m, 1H), 3.14-3.17 (m, 1H), 3.04 (ddd, J = 3.95, 7.83, 11.36 Hz, 1H), 2.68-2.89 (m, 1H), 2.09-2.09 (m, 2H), 2.06 (d, J = 9.79 Hz, 2H), 1.78-1.87 (m, 3H), 1.62-1.69 (m, 2H), 1.37-1.45 (m, 2H), 1.23-1.33 (m, 2H), 1.06 (d, J = 5.74 Hz, 3H), 0.93 (d, J = 6.12 Hz, 3H′); LCMS (+ESI) 402 (M⁺). |
| 41 | 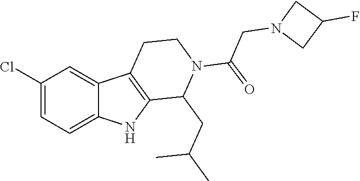 [SMILES: CC(C)CC1c2[nH]c3ccc(Cl)cc3c2CCN1C(=O)CN1CC(F)C1] | ¹H NMR (400 MHz, CD₃OD) δ 7.25-7.26 (m, 1H), 7.12-7.15 (m, 1H), 6.90-6.93 (m, 1H), 5.69-5.72 (m, 1H), 4.94-5.15 (m, 1H), 3.91-3.98 (m, 1H), 3.63-3.71 (m, 2H), 3.40-3.63 (m, 2H), 3.23-3.40 (m, 3H), 2.58-2.79 (m, 2H), 1.68-1.76 (m, 1H), 1.50-1.63 (m, 2H), 1.00 (d, J = 6.09 Hz, 3H), 0.86 (d, J = 6.34 Hz, 3H); LCMS (+ESI) (M⁺). |
| 42 | 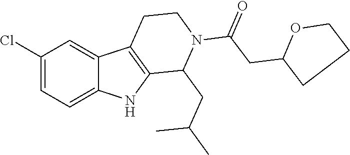 | ¹H NMR (600 MHz, CD₂Cl₂) δ 8.05-8.18 (m, 1H), 7.27-7.33 (m, 1H), 7.15-7.18 (m, 1H), 6.97-7.01 (m, 1H), 5.78 (ddd, J = 4.52, 10.00, 14.56 Hz, 1H), 4.15-4.21 (m, 1H), 4.00 (dt, J = 5.22, 13.62 Hz, 1H), 3.77 (q, J = 7.25 Hz, 1H), 3.62 (quin, J = 7.41 Hz, 1H), 3.33-3.42 (m, 1H), 2.26-2.86 (m, 4H), 1.99-2.09 (m, 1H), 1.76-1.85 (m, 2H), 1.65-1.73 (m, 1H), 1.56-1.63 (m, 1H), 1.45-1.55 (m, 2H), 0.94 (d, J = 6.21 Hz, 3H), 0.87 (d, J = 6.68 Hz, 3H); LCMS (+ESI) 378 (M⁺). |
| 43 | 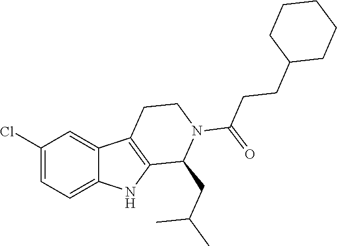 [SMILES: CC(C)C[C@H]1c2[nH]c3ccc(Cl)cc3c2CCN1C(=O)CCC1CCCCC1] | ¹H NMR (400 MHz, CD₃OD) δ 7.38 (d, J = 1.76 Hz, 1H), 7.25 (d, J = 8.60 Hz, 1H), 7.03 (dd, J = 1.98, 8.63 Hz, 1H), 5.83-5.88 (m, 1H), 4.13 (br. s., 1H), 3.50-3.59 (m, 1H), 2.75-2.82 (m, 2H), 2.78 (d, J = 4.52 Hz, 1H), 2.57-2.67 (m, 1H), 1.52-1.88 (m, 10H), 0.92-139 (m, 12H); LCMS (+ESI) 401 (M⁺). |
| 44 | 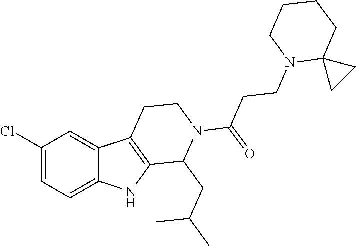 [SMILES: CC(C)CC1c2[nH]c3ccc(Cl)cc3c2CCN1C(=O)CCN1CCCCC12CC2] | ¹H NMR (400 MHz, CDCl₃) δ 7.88 (s, 1H), 7.45 (m, 1H), 7.25 (m, 1H), 7.15 (m, 1H), 7.15 (m, 1H), 5.85 (m, 1H), 4.00-4.10 (m, 1H), 3.40-3.41 (m, 1H), 2.50-3.20 (m, 8H), 1.30-1.80 (m, 9H), 1.08-1.12 (m, 3H), 0.95-0.97 (m, 3H), 0.45 (m, 2H), 0.35 (m, 2H), ; LCMS (+ESI) 428 (M⁺). |
| 45 | 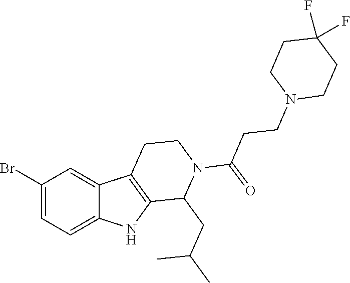 [SMILES: CC(C)CC1c2[nH]c3ccc(Br)cc3c2CCN1C(=O)CCN1CCC(F)(F)CC1] | LCMS (+APCI) 482 (M⁺) |
| 46 | 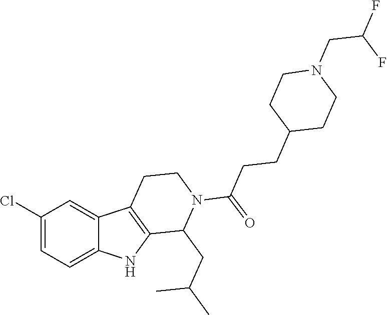 [SMILES: CC(C)CC1c2[nH]c3ccc(Cl)cc3c2CCN1C(=O)CCC1CCN(CC(F)F)CC1] | ¹H NMR (600 MHz, CD₃OD) δ 7.35-7.37 (m, 1H), 7.23-7.25 (m, 1H), 7.00-7.03 (m, 1H), 5.86-6.07 (m, 1H), 5.82-5.85 (m, 1H), 4.13-4.18 (m, 1H), 3.51-3.57 (m, 1H), 2.94-2.99 (m, 2H), 2.45-2.83 (m, 8H), 2.16-2.22 (m, 3H), 1.56-1.85 (m, 4H), 1.25-1.38 (m, 3H), 1.08-1.12 (m, 3H), 0.95-0.97 (m, 3H); LCMS (+ESI) 466 (M⁺). |
| 47 | 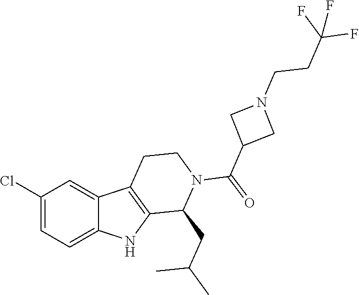 [SMILES: CC(C)C[C@H]1c2[nH]c3ccc(Cl)cc3c2CCN1C(=O)C1CN(CCC(F)(F)F)C1] | ¹H NMR (400 MHz, DMSO-d₆) δ 11.09 (s, 1H), 7.40 (d, J = 1.59 Hz, 1H), 7.30 (d, J = 8.50 Hz, 1H), 7.02 (dd, J = 1.86, 8.53 Hz, 1H), 5.68 (d, J = 8.31 Hz, 1H), 3.79 (dd, J = 4.40, 14.24 Hz, 1H), 3.46-3.65 (m, 3H), 3.31-3.44 (m, 2H), 3.13 (t, J = 6.08 Hz, 2H), 2.58-2.71 (m, 2H), 2.19-2.33 (m, 2H), 1.67-1.79 (m, 1H), 1.52-1.64 (m, 2H), 1.04 (d, J = 5.62 Hz, 3H), 0.90 (d, J = 5.81 Hz, 3H); LCMS (+ESI) 442 (M⁺). |
| 48 | 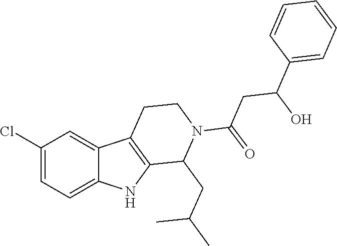 [SMILES: CC(C)CC1c2[nH]c3ccc(Cl)cc3c2CCN1C(=O)CC(O)c1ccccc1] | ¹H NMR (400 MHz, CD₃OD) δ 7.16-7.45 (m, 7H), 6.98-7.02 (m, 1H), 5.82-5.87 (m, 1H), 5.12-5.17 (m, 1H), 4.16-4.23 (m, 1H), 3.44-3.53 (m, 1H), 3.01-3.08 (m, 1H), 2.49-2.95 (m, 3H), 1.58-1.86 (m, 3H), 1.09-1.11 (m, 3H), 0.94-0.97 (m, 3H); LCMS (+ESI) 411 (M⁺). |
| 49 | 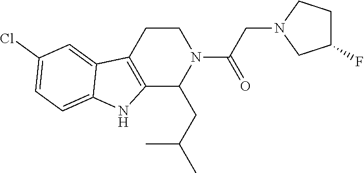 [SMILES: CC(C)CC1c2[nH]c3ccc(Cl)cc3c2CCN1C(=O)CN1CC[C@H](F)C1] | ¹H NMR (400 MHz, CD₃OD) δ 7.25 (s, 1H), 7.15 (d, J = 8.0 Hz, 1H), 7.13 (dd, J = 2.0, 8.0 Hz, 1H), 5.73 (dd, J = 2.0, 8.2 Hz, 1H), 5.10-5.18 (m, 2H), 4.18 (dd, J = 4.0, 12.0 Hz, 1H), 3.42-3.51 (m, 3H), 2.61-2.95 (m, 5H), 2.42-2.50 (m, 1H), 1.81-2.19 (m, 2H), 1.75 (t, J = 8.0 Hz, 1H), 1.52-1.54 (m, 2H), 1.15 (d, J = 8.0 Hz, 3H), 0.92 (d, J = 8.0 Hz, 3H), 0.92 (d, J = 8.0 Hz, 3H); LCMS (+ESI) 392 (M⁺). |
| 50 | 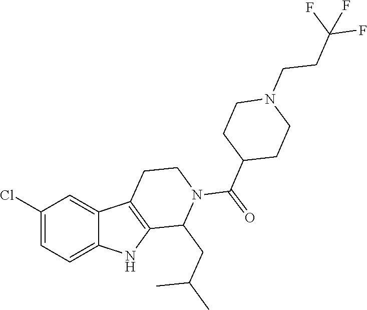 [SMILES: CC(C)CC1c2[nH]c3ccc(Cl)cc3c2CCN1C(=O)C1CCN(CCC(F)(F)F)CC1] | ¹H NMR (400 MHz, DMSO-d₆) δ 11.08 (s, 1H), 7.41 (d, J = 2.07 Hz, 1H), 7.27-7.31 (m, 1H), 7.00-7.04 (m, 1H), 5.68-5.79 (m, 1H), 4.14 (dd, J = 3.45, 14.18 Hz, 1H), 3.31-3.47 (m, 1H), 2.88 (d, J = 3.23, 10.95 Hz, 2H), 2.62-2.76 (m, 3H), 2.32-2.48 (m, 4H), 2.05 (q, J = 11.77 Hz, 2H), 1.49-1.79 (m, 7H), 1.01 (d, J = 5.96 Hz, 3H), 0.89 (d, J = 6.21 Hz, 3H); LCMS (+ESI) 470 (M⁺). |
| 51 | 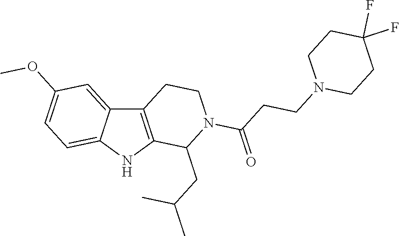 [SMILES: COc1ccc2[nH]c3c(c2c1)CCN(C(=O)CCN1CCC(F)(F)CC1)C3CC(C)C] | ¹H NMR (400 MHz, CD₂Cl₂) δ 7.75 (s, 1H), 7.11 (dd, J = 0.31, 8.5 Hz, 1H), 6.82 (d, J = 2.07 Hz, 1H), 6.69 (dd, J = 2.1, 8.5 Hz, 1H), 5.65-5.80 (m, 1H), 3.91-3.98 (m, 1H), 3.34-3.47 (m, 1H), 2.40-2.79 (m, 10H), 1.85-1.99 (m, 4H), 1.60-1.70 (m, 1H), 1.40-1.51 (m, 2H), 0.98 (d, J = 6.0 Hz, 3H), 0.87 (d, J = 6.3 Hz, 3H); LCMS (+ESI) 434 (M⁺). |
| 52 | 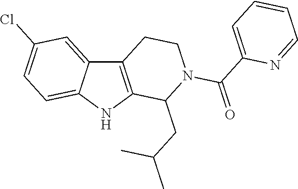 [SMILES: CC(C)CC1c2[nH]c3ccc(Cl)cc3c2CCN1C(=O)c1ccccn1] | LCMS (+APCI) 368 (M⁺) |
| 53 | 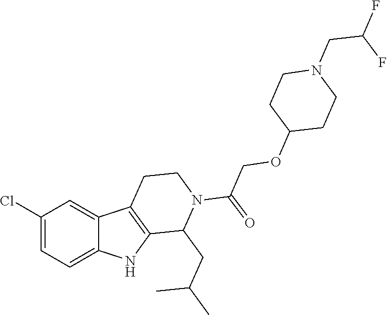 [SMILES: CC(C)CC1c2[nH]c3ccc(Cl)cc3c2CCN1C(=O)COC1CCN(CC(F)F)CC1] | ¹H NMR (400 MHz, DMSO-d₆) δ 11.09 (s, 1H), 7.41 (d, J = 2.07 Hz, 1H), 7.30 (d, J = 8.85 Hz, 1H), 7.03 (dd, J = 2.10, 8.56 Hz, 1H), 5.93-6.25 (m, 1H), 5.61-5.66 (m, 1H), 4.25 (s, 2H), 4.06 (dd, J = 4.64, 14.18 Hz, 1H), 3.37 (tdd, J = 4.30, 8.60, 12.74 Hz, 2H), 2.63-2.82 (m, 6H), 2.21-2.30 (m, 2H), 1.40-1.88 (m, 7H), 1.04 (d, J = 5.90 Hz, 3H), 0.90 (d, J = 6.15 Hz, 3H); LCMS (+ESI) 468 (M⁺). |
| 54 | 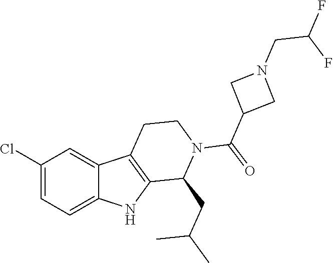 [SMILES: CC(C)C[C@H]1c2[nH]c3ccc(Cl)cc3c2CCN1C(=O)C1CN(CC(F)F)C1] | ¹H NMR (400 MHz, DMSO-d₆) δ 11.09 (s, 1H), 7.40 (d, J = 2.07 Hz, 1H), 7.30 (d, J = 8.60 Hz, 1H), 7.02 (dd, J = 2.07, 8.53 Hz, 1H), 5.93 (s, 1H), 5.68 (dd, J = 2.51, 10.42 Hz, 1H), 3.74-3.81 (m, 1H), 3.56-3.69 (m, 3H), 3.30-3.50 (m, 2H), 2.76 (dt, J = 4.17, 16.17 Hz, 2H), 2.65 (d, J = 3.83 Hz, 2H), 1.73 (d, J = 9.91 Hz, 1H), 1.54-1.63 (m, 2H), 1.04 (d, J = 5.77 Hz, 3H), 0.90 (d, J = 6.09 Hz, 3H); LCMS (+ESI) 410 (M⁺). |
| 55 | 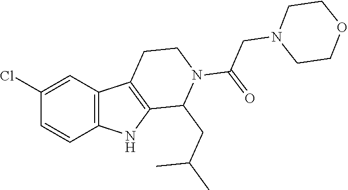 [SMILES: CC(C)CC1c2[nH]c3ccc(Cl)cc3c2CCN1C(=O)CN1CCOCC1] | ¹H NMR (600 MHz, CD₂Cl₂) δ 8.08 (s, 1H), 7.34 (s, 1H), 7.16 (d, J = 8.1 Hz, 1H), 6.99 (dd, J = 2.0, 8.0 Hz, 1H), 5.71 (dd, J = 2.0, 8.2 Hz, 1H), 4.19 (dd, J = 4.0, 12.0 Hz, 1H), 3.58-3.61 (m, 4H), 3.41-3.48 (m, 1H), 3.13-3.22 (dd, J = 8.5, 20.1 Hz, 2H), 2.80-2.85 (m, 1H), 2.62-2.65 (m, 1H), 2.39-2.42 (m, 4H), 1.75 (m, 1H), 1.52-1.54 (m, 2H), 1.15 (d, J = 8.0 Hz, 3H), 0.92 (d, J = 8.0 Hz, 3H); LCMS (+ESI) 390 (M⁺). |
| 56 | 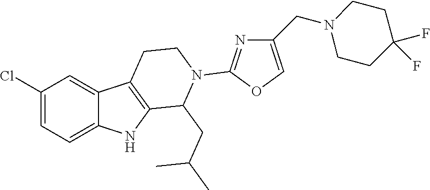 [SMILES: CC(C)CC1c2[nH]c3ccc(Cl)cc3c2CCN1c1nc(CN2CCC(F)(F)CC2)co1] | ¹H NMR (400 MHz, CDCl₃) δ 7.88 (s, 1H), 7.42 (m, 1H), 7.25 (m, 1H), 7.15 (m, 2H), 5.35 (m, 1H), 4.35 (m, 1H), 3.30-3.40 (m, 3H), (m, 1H), 2.60 (m, 5H), 1.80-2.00 (m, 6H), 1.60 (m, 1H), 1.12 (m, 3H), 0.95-0.97 (m, 3H); LCMS (+ESI) 463 (M⁺). |
| 57 | 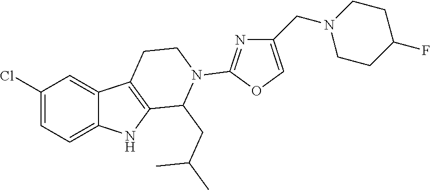 [SMILES: CC(C)CC1c2[nH]c3ccc(Cl)cc3c2CCN1c1nc(CN2CCC(F)CC2)co1] | LCMS (+APCI) 445 (M⁺) |
| 58 | 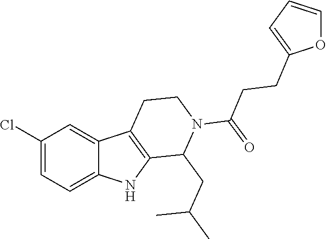 [SMILES: CC(C)CC1c2[nH]c3ccc(Cl)cc3c2CCN1C(=O)CCc1ccco1] | ¹H NMR (400 MHz, CD₂Cl₂) δ 8.50 (s, 1H), 7.30 (s, 1H), 7.22 (s, 1H), 7.12 (d, J = 8.0 Hz, 1H), 6.97 (dd, J = 2.0, 8.5 Hz, 1H), 6.19 (d, J = 4.0 Hz, 1H), 5.95 (d, J = 4.0 Hz, 1H), 5.78 (dd, J = 4.0, 8.2 Hz, 1H), 3.92-3.99 (m, 1H), 3.82 (m, 1H), 3.36 (m, 1H), 2.91-2.97 (m, 2H), 2.61-2.68 (m, 3H), 1.62-2.68 (m, 3H), 1.62-1.68 (m, 1H), 1.46-1.53 (m, 2H), 0.85 (d, J = 6.4 Hz, 3H), 0.81 (d, J = 6.5 Hz, 3H); LCMS (+ESI) 385 (M⁺). |
| 59 | 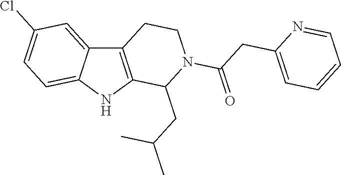 [SMILES: CC(C)CC1c2[nH]c3ccc(Cl)cc3c2CCN1C(=O)Cc1ccccn1] | LCMS (+APCI) 382 (M⁺) |
| 60 | 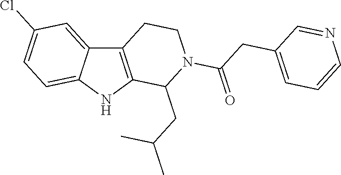 [SMILES: CC(C)CC1c2[nH]c3ccc(Cl)cc3c2CCN1C(=O)Cc1cccnc1] | LCMS (+APCI) 382 (M⁺) |
| 61 | 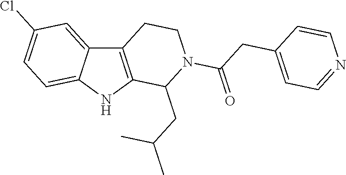 [SMILES: CC(C)CC1c2[nH]c3ccc(Cl)cc3c2CCN1C(=O)Cc1ccncc1] | LCMS (+APCI) 382 (M⁺) |
| 62 | 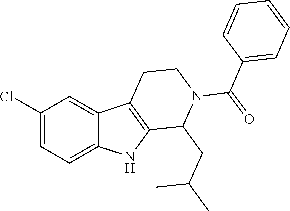 [SMILES: CC(C)CC1c2[nH]c3ccc(Cl)cc3c2CCN1C(=O)c1ccccc1] | ¹H NMR (400 MHz, CDCl₃) δ 8.21 (s, 1H), 7.45 (m, 6H), 7.19 and 7.09 (apparent t, J = 8.1 Hz, 2H), 6.02 (m, 1H), 3.90-3.94 (m, 1H), 3.45-3.50 (m, 1H), 2.58-2.64 (m, 1H), 1.83-1.93 (m, 2H), 1.68-1.74 (m, 1H), 1.60 (s, 1H), 1.10 (d, J = 4.2 Hz, 3H), 1.04 (d, J = 4.2 Hz, 3H); LCMS (+ESI) 367 (M⁺). |
| 63 | 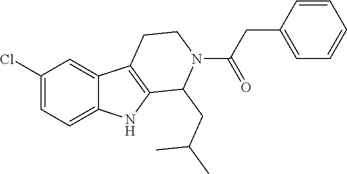 [SMILES: CC(C)CC1c2[nH]c3ccc(Cl)cc3c2CCN1C(=O)Cc1ccccc1] | ¹H NMR (400 MHz, CDCl₃) δ 8.05 (s, 1H), 7.24-7.44 (m, 6H), 7.17 (apparent t, J = 8.2 Hz, 2H), 7.06 (apparent t, J = 8.2 Hz, 2H), 5.89 (m, 1H), 4.04-4.09 (m, 1H), 3.86 (d, J = 4.1 Hz, 1H), 3.39-3.45 (m, 1H), 2.54-2.59 (m, 1H), 2.38-2.47 (m, 1H), 1.68-1.77 (m, 2H), 1.53-1.60 (m, 3H), 1.04 (d, J = 4.2 Hz, 3H), 0.95 (d, J = 4.2 Hz, 3H); LCMS (+ESI) 381 (M⁺). |
| 64 | 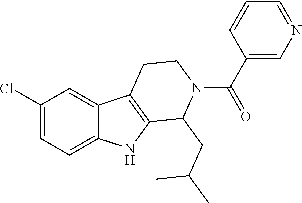 [SMILES: CC(C)CC1c2[nH]c3ccc(Cl)cc3c2CCN1C(=O)c1cccnc1] | ¹H NMR (400 MHz, CDCl₃) δ 8.72 (s, 2H), 8.22 (s, 1H), 7.72-7.79 (m, 1H), 7.37-7.42 (m, 2H), 7.18-7.28 (m, 1H), 7.05-7.10 (m, 1H), 5.95-6.00 (m, 1H), 3.39-3.49 (m, 1H), 2.47-2.80 (m, 2H), 1.57-2.00 (m, 4H), 0.95-1.14 (m, 6H); LCMS (+ESI) 368 (M⁺). |
| 65 | 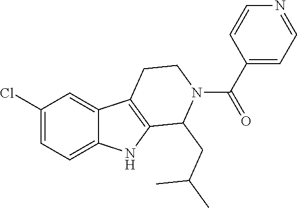 [SMILES: CC(C)CC1c2[nH]c3ccc(Cl)cc3c2CCN1C(=O)c1ccncc1] | ¹H NMR (400 MHz, DMSO-d₆) δ 8.65-8.72 (m, 2H), 7.25-7.49 (m, 5H), 6.99-7.08 (m, 1H), 5.95-6.00 (m, 1H), 3.43-3.60 (m, 1H), 2.44-2.70 (m, 3H), 1.57-2.00 (m, 3H), 0.90-1.12 (m, 6H); LCMS (+APCI) 368 (M⁺). |
| 66 | 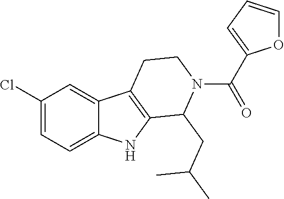 [SMILES: CC(C)CC1c2[nH]c3ccc(Cl)cc3c2CCN1C(=O)c1ccco1] | ¹H NMR (400 MHz, CDCl₃) δ 7.95-8.01 (m, 1H), 7.38-7.54 (m, 3H), 7.19-7.21 (m, 1H), 7.12-7.10 (m, 2H), 5.82-5.90 (m, 1H), 3.50-3.62 (m, 1H), 2.70-3.00 (m, 2H), 1.52-1.95 (m, 4H), 1.00-1.12 (m, 6H); LCMS (+APCI) 357 (M⁺). |
| 67 | 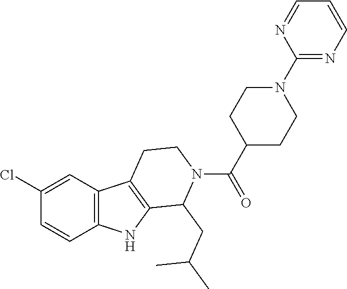 [SMILES: CC(C)CC1c2[nH]c3ccc(Cl)cc3c2CCN1C(=O)C1CCN(c2ncccn2)CC1] | ¹H NMR (400 MHz, CD₃OD) δ 8.28 (d, J = 4.0 Hz, 2H), 7.36 (s, 1H), 7.22 (d, J = 8.0 Hz, 1H), 7.02 (dd, J = 2.0, 8.0 Hz, 1H), 6.55 (t, J = 4.0 Hz, 1H), 5.83 (dd, J = 2.0, 8.2 Hz, 1H), 4.75-4.79 (m, 2H), 4.18 (dd, J = 4.0, 12.0 Hz, 1H), 3.53-3.61 (m, 1H), 3.02-3.31 (m, 3H), 2.80-2.85 (m, 2H), 1.63-1.86 (m, 7H), 1.14 (d, J = 8.0 Hz, 3H), 0.94 (d, J = 8.0 Hz, 3H); LCMS (+ESI) 452 (M⁺). |
| 68 | 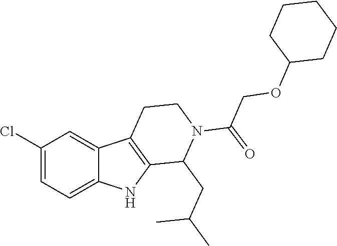 [SMILES: CC(C)CC1c2[nH]c3ccc(Cl)cc3c2CCN1C(=O)COC1CCCCC1] | ¹H NMR (400 MHz, CDCl₃) δ 7.86 (bs, 1H), 7.40 (d, J = 1.8 Hz, 1H), 7.21 (dd, J = 8.6, 0.4 Hz, 1H), 7.11 (dd, J = 8.6, 2.0 Hz, 1H), 5.78 (m, 1H), 4.26 (d, J = 3.7 Hz, 2H), 4.23 (m, 1H), 3.43 (m, 2H), 2.86 (m, 1H), 2.71 (m, 1H), 1.93 (m, 2H), 1.74 (m, 4H), 1.52 (m, 2H), 1.26 (m, 6H), 1.08 (d, J = 6.3 Hz, 3H), 0.99 (d, J = 6.6 Hz, 3H). LCMS (+ESI) 403 (M⁺). |
| 69 | 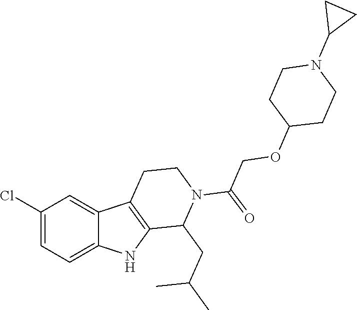 [SMILES: CC(C)CC1c2[nH]c3ccc(Cl)cc3c2CCN1C(=O)COC1CCN(C2CC2)CC1] | ¹H NMR (400 MHz, CDCl₃) δ 7.83 (bs, 1H), 7.40 (d, J = 2.0 Hz, 1H), 7.22 (dd, J = 0.44, 8.6 Hz, 1H), 7.10 (dd, J = 8.6, 2.0 Hz, 1H), 5.78 (dd, J = 4.8, 9.3 Hz, 1H), 4.09-4.40 (m, 3H), 3.30- 3.58 (m, 2H), 2.77-3.01 (m, 3H), 2.71 (dd, J = 3.3, 15.4 Hz, 1H), 2.32 (bs, 2H), 1.84-1.99 (m, 2H), 1.68-1.84 (m, 2H), 1.42-1.67 (m, 8H), 0.92-1.14 (m, 6H), 0.24-0.57 (m, 4H). LCMS (+ESI) 444 (M⁺). |
| 70 | 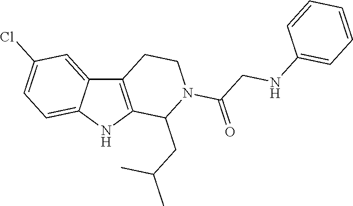 [SMILES: CC(C)CC1c2[nH]c3ccc(Cl)cc3c2CCN1C(=O)CNc1ccccc1] | ¹H NMR (400 MHz, CD₃OD) δ 7.39 (s, 1H), 7.26 (d, J = 8.1 Hz, 1H), 7.15 (t, J = 4.3 Hz, 1H), 7.02-7.05 (m, 1H), 6.61-6.71 (m, 3H), 5.82-5.88 (m, 1H), 5.32-5.38 (m, 1H), 3.50-3.62 (m, 1H), 4.02-4.27 (m, 2H), 3.51-3.62 (m, 1H), 2.70-2.95 (m, 1H), 2.21-2.41 (m, 1H), 2.01-2.11 (m, 1H), 1.83-1.92 (m, 1H), 1.60-1.72 (m, 1H), 1.25-1.42 (m, 1H), 0.88-1.17 (m, 6H); LCMS (+APCI) 396 (M⁺). |
| 71 | 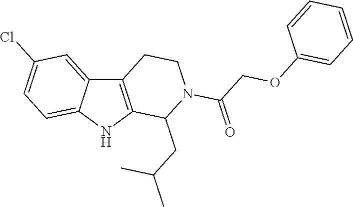 [SMILES: CC(C)CC1c2[nH]c3ccc(Cl)cc3c2CCN1C(=O)COc1ccccc1] | LCMS (+APCI) 397 (M⁺) |
| 72 | 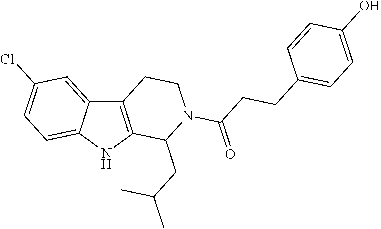 [SMILES: CC(C)CC1c2[nH]c3ccc(Cl)cc3c2CCN1C(=O)CCc1ccc(O)cc1] | LCMS (+APCI) 411 (M⁺) |
| 73 | 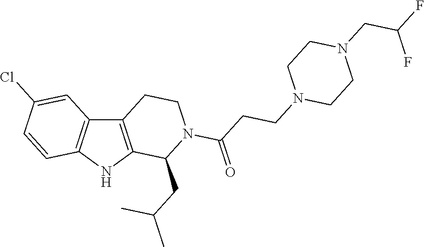 [SMILES: CC(C)C[C@H]1c2[nH]c3ccc(Cl)cc3c2CCN1C(=O)CCN1CCN(CC(F)F)CC1] | ¹H NMR (400 MHz, DMSO-d₆) δ 11.08 (s, 1H), 7.41 (d, J = 1.94 Hz, 1H), 7.30 (d, J = 8.53 Hz, 1H), 7.02 (dd, J = 2.07, 8.53 Hz, 1H), 5.92-6.32 (m, 1H), 5.65-5.80 (m, 1H), 4.01-4.17 (m, 1H), 3.38 (br. s., 1H), 2.62-2.79 (m, 5H), 2.28-2.61 (m, 11H), 1.74 (t, J = 10.26 Hz, 1H), 1.52-1.66 (m, 2H), 1.03 (d, J = 5.96 Hz, 3H), 0.89 (d, J = 6.27 Hz, 3H); LCMS (+ESI) 468 (M⁺). |
| 74 | 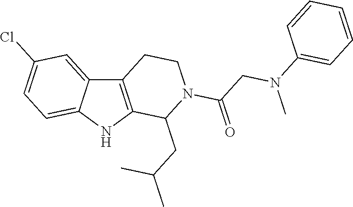 [SMILES: CC(C)CC1c2[nH]c3ccc(Cl)cc3c2CCN1C(=O)CN(C)c1ccccc1] | LCMS (+APCI) 411 (M⁺) |
| 75 | 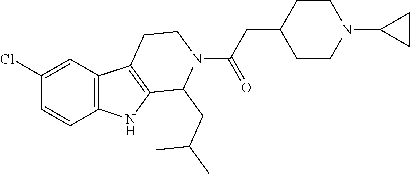 [SMILES: CC(C)CC1c2[nH]c3ccc(Cl)cc3c2CCN1C(=O)CC1CCN(C2CC2)CC1] | ¹H NMR (400 MHz, CD₃OD) δ 7.25 (d, J = 4.0 Hz, 2H), 7.15 (d, J = 8.0 Hz, 1H), 6.90 (dd, J = 2.0, 8.0 Hz, 1H), 5.77 (dd, J = 2.0, 8.2 Hz, 1H), 4.08 (dd, J = 4.0, 12.0 Hz, 1H), 3.41-3.45 (m, 1H), 2.93-2.96 (m, 2H), 2.65-2.67 (m, 2H), 2.30 (m, 1H), 2.42 (m, 1H), 2.16 (m, 2H), 1.54-1.75 (m, 7H), 1.18-1.22 (m, 2H), 1.01 (d, J = 8.0 Hz, 3H), 0.85 (d, J = 8.0 Hz, 3H), 0.49-0.72 (m, 4H); LCMS (+ESI) 428 (M⁺). |
| 76 | 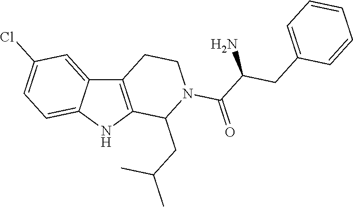 [SMILES: CC(C)CC1c2[nH]c3ccc(Cl)cc3c2CCN1C(=O)[C@@H](N)Cc1ccccc1] | LCMS (+ESI) 410 (M⁺) |
| 77 | 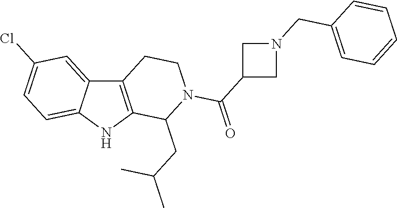 [SMILES: CC(C)CC1c2[nH]c3ccc(Cl)cc3c2CCN1C(=O)C1CN(Cc2ccccc2)C1] | LCMS (+ESI) 436 (M⁺) |
| 78 | 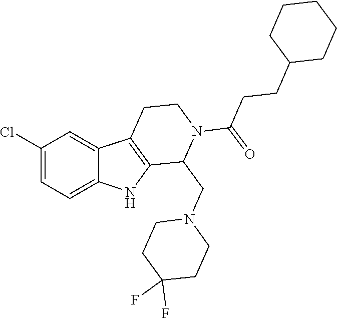 [SMILES: O=C(CCC1CCCCC1)N1CCc2c([nH]c3ccc(Cl)cc23)C1CN1CCC(F)(F)CC1] | ¹H NMR (400 MHz, CDCl₃) δ 8.91 (s, 1H), 7.44 (s, 1H), 7.27 and 7.12 (d, J = 8.0 Hz, 2H), 5.59 (dd, J = 2.0, 8.1 Hz, 1H), 4.13 (dt, J = 1.2, 9.1 Hz, 1H), 3.24-3.33 (m, 1H), 2.90-2.99 (m, 2H), 2.59-2.80 (m, 4H), 2.41-2.49 (m, 2H), 2.00-2.14 (m, 4H), 1.55-1.79 (m, 8H), 1.10-1.36 (m, 3H), and 0.88-1.00 (m, 2H); LCMS (+ESI) 478 (M⁺). |
| 79 | 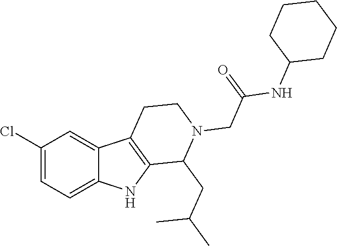 [SMILES: CC(C)CC1c2[nH]c3ccc(Cl)cc3c2CCN1CC(=O)NC1CCCCC1] | ¹H NMR (400 MHz, CDCl₃) δ 7.77 (bs, 1H), 7.43 (d, J = 2.1 Hz, 1H), 7.30 (d, J = 8.3 Hz, 1H), 7.20 (dd, J = 0.44, 8.5 Hz, 1H), 7.09 (dd, J = 2.0, 8.6 Hz, 1H), 4.12 (q, J = 7.1 Hz, 1H), 3.73-3.91 (m, 2H), 3.64 (t, J = 6.8 Hz, 2H), 3.22-3.29 (m, 2H), 3.19 (dt, J = 4.9, 9.2 Hz, 2H), 3.07-3.14 (m, 2H), 2.75-2.95 (m, 4H), 2.04 (s, 1H), 1.80-2.00 (m, 4H), 1.59-1.67 (m, 2H), 1.51-1.59 (m, 5H), 1.33-1.49 (m, 3H), 1.11-1.31 (m, 5H), 1.00 (dd, J = 5.5, 6.5 Hz, 6H). LCMS (+ESI) 402 (M⁺). |
| 80 | 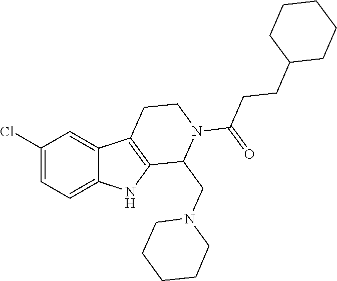 [SMILES: O=C(CCC1CCCCC1)N1CCc2c([nH]c3ccc(Cl)cc23)C1CN1CCCCC1] | ¹H NMR (400 MHz, CDCl₃) δ 9.39 (s, 1H), 7.42 (s, 1H), 7.25 (apparent t, J = 8.2 Hz, 2H) 7.09 (apparent t, J = 8.2 Hz, 2H), 5.60 (dd, J = 2.2, 8.2 Hz, 1H), 4.09 (dt, J = 1.4, 9.6 Hz, 1H), 3.20-3.30 (m, 1H), 2.70-2.95 (m, 4H), 2.50-2.70 (m, 2H), 2.30-2.50 (m, 3H), 1.48-1.79 (m, 13H), 1.05-1.35 (m, 5H), 0.85-1.01 (m, 2H); LCMS (+ESI) 442 (M⁺). |
| 81 | 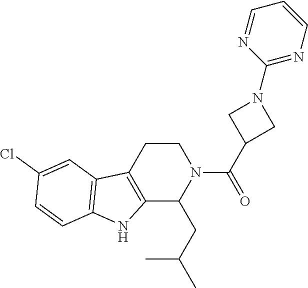 [SMILES: CC(C)CC1c2[nH]c3ccc(Cl)cc3c2CCN1C(=O)C1CN(c2ncccn2)C1] | ¹H NMR (400 MHz, CD₃OD) δ 8.30 (d, J = 4.0 Hz, 2H), 7.36 (s, 1H), 7.22 (d, J = 8.0 Hz, 1H), 7.02 (dd, J = 2.0, 8.0 Hz, 1H), 6.55 (t, J = 4.0 Hz, 1H), 5.83 (dd, J = 2.0, 8.2 Hz, 1H), 4.25-4.42 (m, 4H), 3.99 (m, 1H), 3.88-3.91 (m, 1H), 3.51-3.56 (m, 1H), 2.74-2.77 (m, 2H), 1.81 (t, J = 8.0 Hz, 1H), 1.65-1.68 (m, 2H), 1.10 (d, J = 8.0 Hz, 3H), 0.96 (d, J = 8.0 Hz, 3H); LCMS (+ESI) 424 (M⁺). |
| 82 | 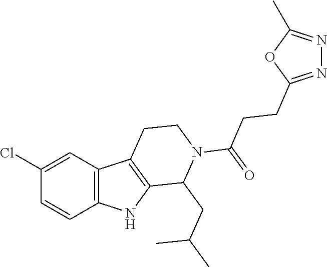 [SMILES: Cc1nnc(CCC(=O)N2CCc3c([nH]c4ccc(Cl)cc34)C2CC(C)C)o1] | ¹H NMR (400 MHz, CD₃OD) δ 7.35 (s, 1H), 7.12 (d, J = 8.0 Hz, 1H), 6.99 (dd, J = 2.0, 8.4 Hz, 1H), 5.78 ((d, J = 4.0 Hz, 1H), 4.16 (dd, J = 4.0, 8.2 Hz, 1H), 3.52-3.57 (m, 1H), 3.12-3.16 (m, 4H), 2.71-2.85 (m, 2H), 2.51 (s, 3H), 1.82 (m, 1H), 1.65 (m, 2H), 1.07 (d, J = 6.4 Hz, 3H), 0.94 (d, J = 6.5 Hz, 3H); LCMS (+ESI) 401 (M⁺). |
| 83 | 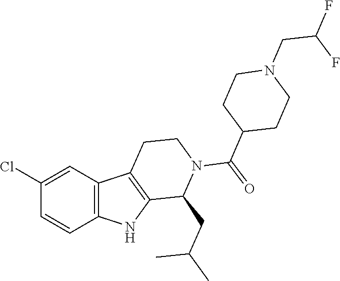 [SMILES: CC(C)C[C@H]1c2[nH]c3ccc(Cl)cc3c2CCN1C(=O)C1CCN(CC(F)F)CC1] | ¹H NMR (600 MHz, DMSO-d₆) δ 11.09 (s, 1H), 7.41 (d, J = 1.60 Hz, 1H), 7.30 (d, J = 8.47 Hz, 1H), 7.02 (dd, J = 1.93, 8.52 Hz, 1H), 5.99-6.22 (m, 1H), 5.71-5.76 (m, 1H), 4.15 (dd, J = 4.33, 14.12 Hz, 1H), 3.40 (t, J = 10.59 Hz, 1H), 2.91 (dd, J = 3.39, 10.92 Hz, 2H), 2.65-2.75 (m, 5H), 2.21-2.32 (m, 2H), 1.51-1.77 (m, 7H), 1.01 (d, J = 5.84 Hz, 3H), 0.89 (d, J = 6.12 Hz, 3H); LCMS (+ESI) 438 (M⁺). |
| 84 | 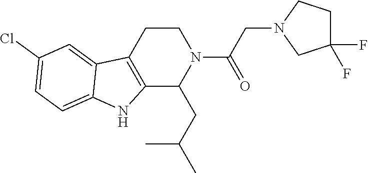 [SMILES: CC(C)CC1c2[nH]c3ccc(Cl)cc3c2CCN1C(=O)CN1CCC(F)(F)C1] | ¹H NMR (600 MHz, CD₃OD) δ 7.34 (s, 1H), 7.24 (d, J = 8.1 Hz, 1H), 7.01 (dd, J = 2.0, 8.0 Hz, 1H), 5.75 (dd, J = 2.0, 8.2 Hz, 1H), 4.19 (dd, J = 4.0, 12.0 Hz, 1H), 3.49-3.54 (m, 3H), 2.91-3.11 (m, 1H), 2.71-2.87 (m, 5H), 2.26-1.28 (m, 2H), 1.83 (m, 1H), 1.66 (m, 2H), 1.11 (d, J = 8.0 Hz, 3H), 0.95 (d, J = 8.0 Hz, 3H); LCMS (+ESI) 410 (M⁺). |
| 85 | 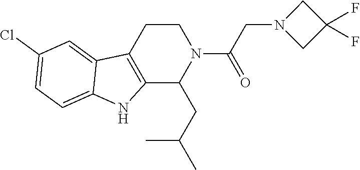 [SMILES: CC(C)CC1c2[nH]c3ccc(Cl)cc3c2CCN1C(=O)CN1CC(F)(F)C1] | ¹H NMR (400 MHz, CD₃OD) δ 7.25 (s, 1H), 7.14 (d, J = 8.2 Hz, 1H), 6.91 (dd, J = 2.0, 8.2 Hz, 1H), 5.67 (dd, J = 2.0, 8.4 Hz, 1H), 3.91-3.94 (dd, J = 4.0, 12.1 Hz, 1H), 3.51-3.70 (m, 4H), 3.37-3.42 (m, 1H), 2.60-2.74 (m, 2H), 1.58 (t, J = 8.0 Hz, 1H), 1.52-1.54 (m, 2H), 1.15 (d, J = 8.0 Hz, 3H), 0.92 (d, J = 8.0 Hz, 3H); LCMS (+ESI) 396 (M⁺). |
| 86 | 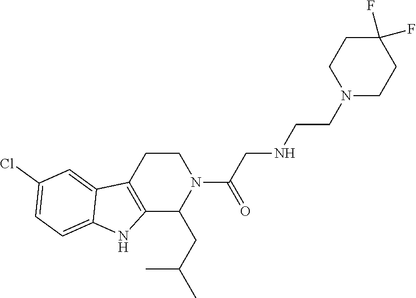 [SMILES: CC(C)CC1c2[nH]c3ccc(Cl)cc3c2CCN1C(=O)CNCCN1CCC(F)(F)CC1] | ¹H NMR (400 MHz, CD₃OD) δ 7.26 (s, 1H), 7.14 (d, J = 2.07 Hz, 1H), 6.92 (dd, J = 2.1, 8.5 Hz, 1H), 5.69-5.72 (m, 1H), 4.01 (d, J = 12.1 Hz, 1H), 4.15 (d, J = 12.1 Hz, 1H), 3.75-3.85 (m, 1H), 3.48-3.52 (m, 1H), 3.04-3.06 (m, 2H), 2.56-2.80 (m, 8H), 1.90-2.04 (m, 4H), 1.81 (t, J = 8.1 Hz, 1H), 1.55-1.65 (m, 2H), 1.02 (d, J = 6.5 Hz, 3H), 0.86 (d, J = 6.4 Hz, 3H); LCMS (+ESI) 467 (M⁺). |
| 87 | 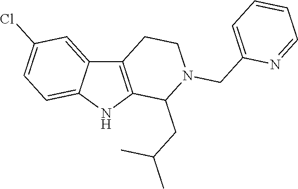 [SMILES: CC(C)CC1c2[nH]c3ccc(Cl)cc3c2CCN1Cc1ccccn1] | ¹H NMR (400 MHz, CDCl₃) δ 8.54 (dq, J = 4.9, 0.9 Hz, 1H), 7.73 (bs, 1H), 7.67 (td, J = 7.6, 1.8 Hz, 1H), 7.53 (d, J = 7.8 Hz, 1H), 7.44 (d, J = 2.0 Hz, 1H), 7.16 (m, 2H), 7.08 (dd, J = 8.6, 2.1 Hz, 1H), 3.86 (dd, J = 28.6, 18.3 Hz, 2H), 3.70 (m, 1H), 3.25 (m, 1H), 3.03 (m, 1H), 2.90 (m, 1H), 2.53 (m, 1H), 2.81 (m, 2H), 1.39 (m, 1H), 0.90 (d, J = 6.7 Hz, 3H), 0.69 (d, J = 6.5 Hz, 3H); LCMS (+ESI) 354 (M⁺). |
| 88 | 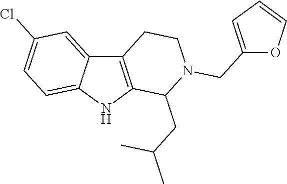 [SMILES: CC(C)CC1c2[nH]c3ccc(Cl)cc3c2CCN1Cc1ccco1] | ¹H NMR (400 MHz, CDCl₃) δ 7.64 (bs, 1H), 7.45 (d, J = 2.0 Hz, 1H), 7.39 (dd, J = 1.8, 0.8 Hz, 1H), 7.21 (d, J = 8.6 Hz, 1H), 7.09 (dd, J = 8.5, 2.0 Hz, 1H), 6.31 (dd, J = 3.1, 1.9 Hz, 1H), 6.17 (d, J = 3.1 Hz, 1H), 3.75 (m, 3H), 3.28 (m, 1H), 3.08 (m, 1H), 2.86 (m, 1H), 2.53 (m, 1H), 1.80 (m, 2H), 1.55 (m, 2H), 1.39 (m, 1H), 1.25 (m, 2H), 0.90 (d, J = 6.6 Hz, 3H), 0.89 (m, 1H), 0.75 (d, J = 6.5 Hz, 3H); LCMS (+ESI) 343 (M⁺). |
| 89 | 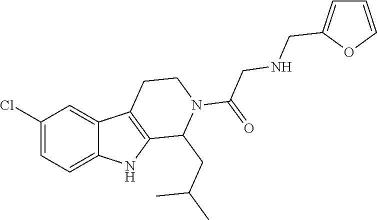 [SMILES: CC(C)CC1c2[nH]c3ccc(Cl)cc3c2CCN1C(=O)CNCc1ccco1] | ¹H NMR (400 MHz, CDCl₃) δ 8.23 (bs, 1H), 7.99 (bs, 1H), 7.37 (m, 3H), 7.21 (d, J = 8.6 Hz, 1H), 7.11 (dd, J = 8.6, 1.9 Hz, 1H), 6.32 (m, 4H), 5.83 (m, 1H), 4.50 (d, J = 5.8 Hz, 2H), 3.84 (m, 3H), 3.60 (dd, J = 44.7, 15.8 Hz, 2H), 3.41 (m, 1H), 2.67 (m, 2H), 1.81-1.55 (m, 4H), 1.09 (d, J = 6.3 Hz, 3H), 0.98 (d, J = 6.5 Hz, 3H); LCMS (+ESI) 400 (M⁺). |
| 90 | 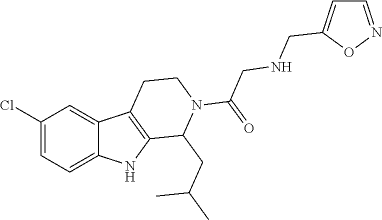 [SMILES: CC(C)CC1c2[nH]c3ccc(Cl)cc3c2CCN1C(=O)CNCc1ccno1] | ¹H NMR (400 MHz, CDCl₃) δ 8.19 (d, J = 1.7 Hz, 1H), 7.85 (bs, 1H), 7.40 (d, J = 1.9 Hz, 1H), 7.23 (d, J = 8.6 Hz, 1H), 7.12 (dd, J = 8.6 Hz, 1H), 7.12 (dd, J = 8.6, 2.0 Hz, 1H), 6.20 (m, 1H), 5.85 (m, 1H), 4.01 (d, J = 2.0 Hz, 2H), 3.86 (m, 1H), 3.70 (dd, J = 50.6, 15.9 Hz, 2H), 3.45 (m, 1H), 2.72 (m, 2H), 1.72-1.55 (m, 4H), 1.10 (d, J = 6.3 Hz, 3H), 0.98 (d, J = 6.5 Hz, 3H); LCMS (+ESI) 401 (M⁺). |
| 91 | 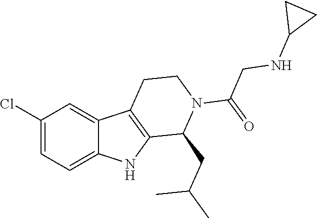 [SMILES: CC(C)C[C@H]1c2[nH]c3ccc(Cl)cc3c2CCN1C(=O)CNC1CC1] | ¹H NMR (400 MHz, DMSO-d₆) δ 11.09 (s, 1H), 7.41 (d, J = 2.02 Hz, 1H), 7.30 (dd, J = 0.37, 8.56 Hz, 1H), 7.03 (d, J = 2.14 Hz, 1H), 5.67-5.73 (m, 1H), 4.03 (dd, J = 4.07, 14.09 Hz, 1H), 3.52-3.57 (m, 1H), 3.44-3.63 (m, 1H), 3.44-3.57 (m, 1H), 2.63-2.82 (m, 3H), 2.13 (tt, J = 3.42, 6.63 Hz, 1H), 1.72-1.80 (m, 1H), 1.54-1.65 (m, 2H), 1.05 (d, J = 5.99 Hz, 3H), 0.90 (d, J = 6.24 Hz, 3H), 0.31-0.34 (m, 2H), 0.22-0.26 (m, 2H); HRLCMS m/z 360.1837 (M⁺). |
| 92 | 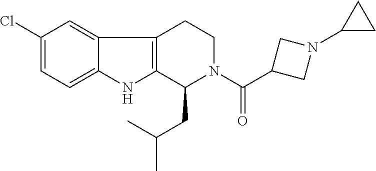 [SMILES: CC(C)C[C@H]1c2[nH]c3ccc(Cl)cc3c2CCN1C(=O)C1CN(C2CC2)C1] | ¹H NMR (400 MHz, CD₃OD) δ 7.40 (s, 1H), 7.28 (dd, J = 0.4, 8.6 Hz, 1H), 7.07 (dd, J = 2.0, 8.6 Hz, 1H), 5.84 (d, J = 6.0 Hz, 1H), 4.44-4.80 (m, 4H), 4.15-4.41 (m, 1H), 3.60 (ddd, J = 4.5, 11.7, 14.2 Hz, 1H), 3.57 (ddd, J = 4.5, 11.7, 14.2 Hz, 1H), 3.11 (m, 1H), 2.75-2.78 (m, 2H), 1.71 (m, 1H), 1.57 (m, 2H), 1.68 (d, J = 6.4 Hz, 3H), 1.30 (d, J = 6.5 Hz, 3H), 1.09-1.14 (m, 4H); LCMS (+ESI) 386 (M⁺). |
| 93 | 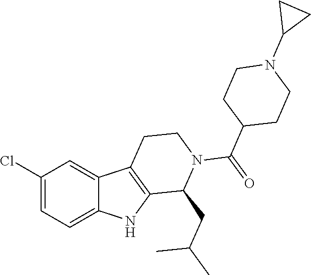 [SMILES: CC(C)C[C@H]1c2[nH]c3ccc(Cl)cc3c2CCN1C(=O)C1CCN(C2CC2)CC1] | ¹H NMR (400 MHz, DMSO-d₆) δ 11.08 (s, 1H), 7.41 (d, J = 1.96 Hz, 1H), 7.29 (d, J = 8.56 Hz, 1H), 7.27-7.31 (m, 1H), 7.02 (dd, J = 2.02, 8.56 Hz, 1H), 5.71-5.76 (m, 1H), 4.15 (dd, J = 3.64, 14.21 Hz, 1H), 3.36-3.45 (m, 1H), 2.89-2.97 (m, 2H), 2.65-2.74 (m, 3H), 2.18-2.30 (m, 2H), 1.63-1.77 (m, 3H), 1.50-1.61 (m, 5H), 1.00 (d, J = 5.93 Hz, 3H), 0.89 (d, J = 6.17 Hz, 3H), 0.36-0.42 (m, 2H), 0.27 (d, J = 2.57 Hz, 2H); LCMS (+ESI) 414 (M⁺). |
| 94 | 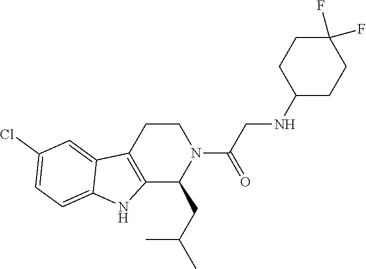 [SMILES: CC(C)C[C@H]1c2[nH]c3ccc(Cl)cc3c2CCN1C(=O)CNC1CCC(F)(F)CC1] | ¹H NMR (400 MHz, CD₃OD) δ 7.34 (s, 1H), 7.22 (dd, J = 0.4, 8.6 Hz, 1H), 6.99 (dd, J = 2.0, 8.6 Hz, 1H), 5.80 (d, J = 4.0 Hz, 1H), 4.03-4.08 (m, 1H), 3.68 (d, J = 20.1 Hz, 1H), 3.55 (d, J = 16.3 Hz, 1H), 3.47 (m, 1H), 2.63-2.82 (m, 3H), 1.68-2.14 (m, 10H), 1.50 (m, 2H), 1.11 (d, J = 8.0 Hz, 3H), 0.95 (d, J = 8.0 Hz, 3H); LCMS (+ESI) 438 (M⁺). |
| 95 | 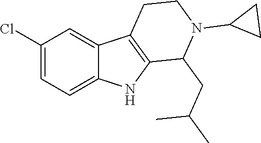 [SMILES: CC(C)CC1c2[nH]c3ccc(Cl)cc3c2CCN1C1CC1] | ¹H NMR (400 MHz, CDCl₃) δ 7.67 (bs, 1H), 7.44 (d, J = 2.0 Hz, 1H), 7.22 (d, J = 8.5 Hz, 1H), 7.09 (dd, J = 8.5, 2.0 Hz, 1H), 3.89 (m, 1H), 3.11 (m, 2H), 2.94 (m, 1H), 2.49 (m, 1H), 2.16 (m, 1H), 1.84 (m, 1H), 1.35 (m, 1H), 0.99 (d, J = 6.5 Hz, 3H), 0.96 (d, J = 6.7 Hz, 3H), 0.52-0.43 (m, 4H); LCMS (+ESI) 303 (M⁺). |
| 96 | 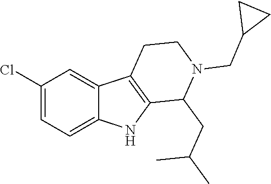 [SMILES: CC(C)CC1c2[nH]c3ccc(Cl)cc3c2CCN1CC1CC1] | ¹H NMR (400 MHz, CDCl₃) δ 7.64 (bs, 1H), 7.42 (d, J = 1.9 Hz, 1H), 7.20 (d, J = 8.6 Hz, 1H), 7.08 (dd, J = 8.5, 2.0 Hz, 1H), 3.95 (dd, J = 9.0, 4.8, 1H), 3.25 (m, 1H), 3.11 (ddd, J = 22.3 5.5, 2.0, 1H), 2.79 (m, 1H), 2.66 (dd, J = 12.7, 5.5 Hz, 1H), 2.46 (ddd, J = 15.7, 4.9, 2.0 Hz, 1H), 2.34 (dd, J = 12.7, 6.7 Hz, 1H), 1.99 (m, 1H), 1.78 (m, 1H), 1.44 (m, 1H), 1.02 (d, J = 6.6 Hz, 3H), 0.99 (d, J = 6.7 Hz, 3H), 0.92 (m, 1H), 0.52 (m, 2H), 0.06 (m, 2H); LCMS (+ESI) 317 (M⁺). |
| 97 | 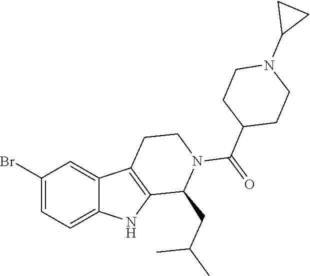 [SMILES: CC(C)C[C@H]1c2[nH]c3ccc(Br)cc3c2CCN1C(=O)C1CCN(C2CC2)CC1] | ¹H NMR (400 MHz, CDCl₃) δ 8.19 (bs, 1H), 7.55 (d, J = 1.8 Hz, 1H), 7.24 (dd, J = 8.6, 1.8 Hz, 1H), 7.17 (d, J = 8.5 Hz, 1H), 5.89 (m, 1H), 4.11 (m, 1H), 3.49 (m, 1H), 3.12 (m, 2H), 2.78 (m, 2H), 2.59 (m, 1H), 2.25 (m, 2H), 1.54-1.91 (m, 9H), 0.98 (d, J = 6.2 Hz, 3H), 0.92 (d, J = 6.4 Hz, 3H), 0.34 (m, 4H); LCMS (+ESI) 458 (M⁺). |
| 98 | 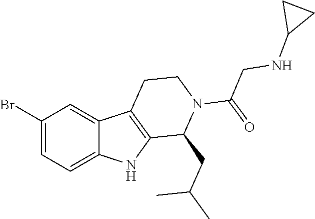 [SMILES: CC(C)C[C@H]1c2[nH]c3ccc(Br)cc3c2CCN1C(=O)CNC1CC1] | ¹H NMR (400 MHz, DMSO-d₆) δ 11.10 (s, 1H), 7.55 (d, J = 1.59 Hz, 1H), 7.26 (d, J = 8.50 Hz, 1H), 7.14 (dd, J = 1.90, 8.50 Hz, 1H), 5.76-5.80 (m, 1H), 4.03 (dd, J = 3.61, 14.49 Hz, 1H), 3.43-3.63 (m, 2H), 3.31-3.40 (m, 2H), 2.66-2.73 (m, 2H), 2.13 (tt, J = 8.29, 6.49 Hz, 1H), 1.72-1.79 (m, 1H), 1.57-1.64 (m, 2H), 1.04 (d, J = 5.81 Hz, 3H), 0.90 (d, J = 6.05 Hz, 3H), 0.31-0.35 (m, 2H), 0.22-0.26 (m, 2H); LCMS (+ESI) 404 (M⁺). |
| 99 | 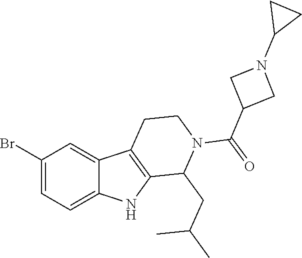 [SMILES: CC(C)CC1c2[nH]c3ccc(Br)cc3c2CCN1C(=O)C1CN(C2CC2)C1] | ¹H NMR (400 MHz, CDCl₃) δ 8.03 (bs, 1H), 7.54 (d, J = 1.9 Hz, 1H), 7.24 (dd, J = 8.6, 1.8 Hz, 1H), 7.19 (d, J = 8.5 Hz, 1H), 5.82 (m, 1H), 3.79 (m, 1H), 3.67 (m, 2H), 3.42-3.52 (m, 4H), 2.69 (m, 2H), 1.85 (m, 1H), 1.75-1.55 (m, 7H), 1.06 (d, J = 6.2 Hz, 3H), 0.98 (d, J = 6.4 Hz, 3H), 0.36 (m, 4H); LCMS (+ESI) 431 (M⁺). |
| 100 | 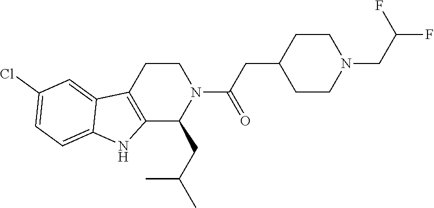 [SMILES: CC(C)C[C@H]1c2[nH]c3ccc(Cl)cc3c2CCN1C(=O)CC1CCN(CC(F)F)CC1] | ¹H NMR (400 MHz, CD₃OD) δ 7.37 (s, 1H), 7.24 (dd, J = 0.4, 8.6 Hz, 1H), 7.04 (dd, J = 2.0, 8.6 Hz, 1H), 5.96-5.98 (m, 1H), 5.83-5.89 (m, 1H), 4.44-4.80 (m, 4H), 4.17-4.31 (m, 1H), 3.33 (ddd, J = 4.5, 11.6, 14.1 Hz, 1H), 2.69-2.99 (m, 6H), 2.54 (dd, J = 8.0, 16.1 Hz, 1H), 2.42 (dd, J = 8.0, 16.1 Hz, 1H), 2.23-2.38 (m, 2H), 1.65-1.86 (m, 6H), 1.38-1.42 (m, 2H), 1.12 (d, J = 6.4 Hz, 3H), 0.97 (d, J = 6.4 Hz, 3H); LCMS (+ESI) 452 (M⁺). |
| 101 | 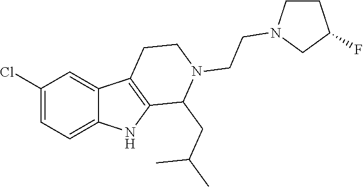 [SMILES: CC(C)CC1c2[nH]c3ccc(Cl)cc3c2CCN1CCN1CC[C@H](F)C1] | ¹H NMR (400 MHz, DMSO-d₆) δ 10.83 (s, 1H), 7.37 (d, J = 2.02 Hz, 1H), 7.27 (d, J = 8.50 Hz, 1H), 6.98 (dd, J = 2.08, 8.50 Hz, 1H), 5.05-5.23 (m, 1H), 3.70-3.76 (m, 1H), 2.53-3.11 (m, 10H), 2.24-2.42 (m, 2H), 1.99-2.15 (m, 1H), 1.40-1.98 (m, 4H), 0.98 (d, J = 6.54 Hz, 3H), 0.90 (d, J = 6.72 Hz, 3H); LCMS (+ESI) 378 (M⁺). |
| 102 | 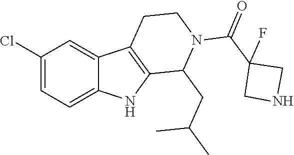 [SMILES: CC(C)CC1c2[nH]c3ccc(Cl)cc3c2CCN1C(=O)C1(F)CNC1] | ¹H NMR (400 MHz, DMSO-d₆) δ 11.14 (s, 1H), 7.42 (d, J = 1.90 Hz, 1H), 7.32 (d, J = 8.56 Hz, 1H), 7.04 (dd, J = 2.02, 8.56 Hz, 1H), 5.63-5.68 (m, 1H), 3.91-4.12 (m, 2H), 3.65-3.84 (m, 3H), 3.43-3.53 (m, 1H), 2.66-2.80 (m, 3H), 1.76-1.85 (m, 1H), 1.56-1.69 (m, 2H), 0.99-1.09 (m, 3H), 0.91 (s, 3H); LCMS (+ESI) 364 (M⁺). |
| 103 | 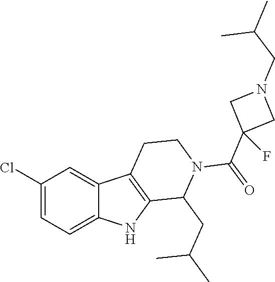 [SMILES: CC(C)CC1c2[nH]c3ccc(Cl)cc3c2CCN1C(=O)C1(F)CN(CC(C)C)C1] | ¹H NMR (400 MHz, DMSO-d₆) δ 11.13 (s, 1H), 7.42 (d, J = 1.90 Hz, 1H), 7.32 (d, J = 8.56 Hz, 1H), 7.04 (dd, J = 2.05, 8.59 Hz, 1H), 5.65 (dd, J = 3.24, 10.39 Hz, 1H), 3.62-3.81 (m, 3H), 3.41-3.55 (m, 3H), 2.63-2.76 (m, 2H), 2.25 (d, J = 6.91 Hz, 2H), 1.74-1.86 (m, 1H), 1.45-1.71 (m, 3H), 1.04 (d, J = 6.11 Hz, 3H), 0.92 (d, J = 6.36 Hz, 3H), 0.83 (d, J = 6.60 Hz, 6H); LCMS (+ESI) 420 (M⁺). |
| 104 | 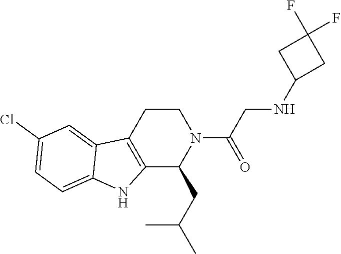 [SMILES: CC(C)C[C@H]1c2[nH]c3ccc(Cl)cc3c2CCN1C(=O)CNC1CC(F)(F)C1] | ¹H NMR (400 MHz, CD₃OD) δ 7.38 (s, 1H), 7.25 (dd, J = 0.4, 8.6 Hz, 1H), 7.04 (dd, J = 2.0, 8.6 Hz, 1H), 5.84 (dd, J = 2.0, 8.6 Hz, 1H), 4.04-4.09 (dd, J = 4.0, 12.1 Hz, 1H), 3.6 (d, J = 16.3 Hz, 1H), 3.56 (d, J = 16.3 Hz, 1H), 3.49-3.52 (m, 1H), 3.25-3.30 (m, 1H), 2.72-2.84 (m, 4H), 2.41-2.45 (m, 2H), 1.86 (t, J = 8.2 Hz, 1H), 1.67-1.72 (m, 2H), 1.13 (d, J = 8.0 Hz, 3H), 0.97 (d, J = 8.0 Hz, 3H); LCMS (+ESI) 410 (M⁺). |
| 105 | 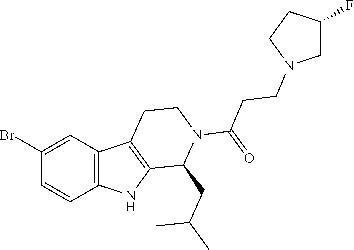 [SMILES: CC(C)C[C@H]1c2[nH]c3ccc(Br)cc3c2CCN1C(=O)CCN1CC[C@H](F)C1] | ¹H NMR (400 MHz, CD₃OD) δ 7.1 (d, J = 4.1 Hz, 1H), 7.14-7.24 (m, 2H), 5.86 (dd, J = 4.3, 8.5 Hz, 1H), 5.12-5.32 (m, 1H), 4.18 (dd, J = 4.3, 14.2 Hz, 1H), 3.56 (ddd, J = 4.5, 11.7, 14.2 Hz, 1H), 2.66-3.34 (m, 9H), 2.17-2.48 (m, 1H), 2.47 (m, 1H), 2.17 (m, 1H), 1.85 (t, J = 8.6 Hz, 1H), 1.66-1.70 (m, 2H), 1.14 (d, J = 6.4 Hz, 3H), 0.97 (d, J = 6.5 Hz, 3H); LCMS (+ESI) 450 (M⁺). |
| 106 | 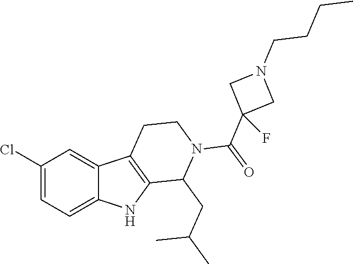 [SMILES: CCCCN1CC(F)(C(=O)N2CCc3c([nH]c4ccc(Cl)cc34)C2CC(C)C)C1] | ¹H NMR (400 MHz, CD₃OD) δ 7.35 (d, J = 1.77 Hz, 1H), 7.24 (dd, J = 0.34, 8.59 Hz, 1H), 7.01 (dd, J = 2.05, 8.59 Hz, 1H), 5.74 (dd, J = 3.06, 10.15 Hz, 1H), 3.74-3.98 (m, 3H), 3.47-3.68 (m, 3H), 2.69-2.80 (m, 2H), 2.52 (t, J = 7.24 Hz, 2H), 1.84 (t, J = 10.00 Hz, 1H), 1.65-1.73 (m, 2H), 1.31-1.39 (m, 5H), 1.10 (d, J = 5.99 Hz, 3H), 0.88-1.00 (m, 6H); LCMS (+ESI) 420 (M⁺). |
| 107 | 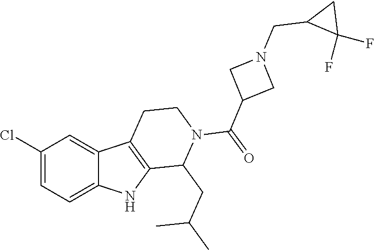 [SMILES: CC(C)CC1c2[nH]c3ccc(Cl)cc3c2CCN1C(=O)C1CN(CC2CC2(F)F)C1] | ¹H NMR (400 MHz, DMSO-d₆) δ 10.86 (s, 1H), 7.17 (s, 1H), 7.07 (d, J = 8.56 Hz, 1H), 6.79 (dd, J = 2.05, 8.53 Hz, 1H), 5.43-5.48 (m, 1H), 3.57 (dd, J = 4.62, 14.15 Hz, 1H), 3.26-3.40 (m, 4H), 2.90-2.98 (m, 2H), 2.66 (s, 1H), 2.10-2.47 (m, 4H), 1.22-1.54 (m, 3H), 0.86 (t, J = 7.00 Hz, 1H), 0.81 (d, J = 5.69 Hz, 3H), 0.67 (d, J = 5.93 Hz, 3H), −0.04-0.12 (m, 1H); LCMS (+ESI) 436 (M⁺). |
| 108 | 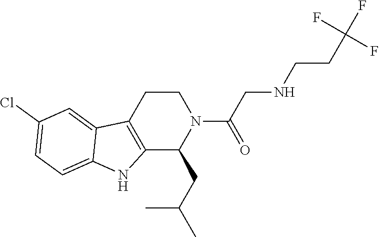 [SMILES: CC(C)C[C@H]1c2[nH]c3ccc(Cl)cc3c2CCN1C(=O)CNCCC(F)(F)F] | ¹H NMR (400 MHz, CD₃OD) δ 7.25 (d, J = 4.1 Hz, 1H), 7.13 (d, J = 8.3 Hz, 1H), 6.91 (dd, J = 2.0, 8.6 Hz, 1H), 5.73 (dd, J = 2.0, 8.6 Hz, 1H), 4.70 (bs, 1H), 3.93-3.98 (dd, J = 4.0, 12.1 Hz, 1H), 3.62 (d, J = 16.2 Hz, 1H), 3.48 (d, J = 16.2 Hz, 1H), 3.37-3.45 (m, 1H), 2.61-2.79 (m, 4H), 2.25-2.36 (m, 2H), 1.75 (t, J = 12.0 Hz, 1H), 1.55-1.60 (m, 2H), 1.11 (d, J = 8.0 Hz, 3H), 0.95 (d, J = 8.0 Hz, 3H); LCMS (+ESI) 416 (M⁺). |
| 109 | 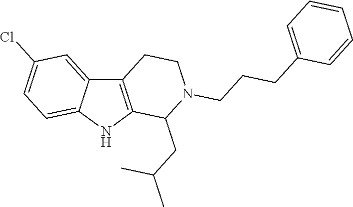 [SMILES: CC(C)CC1c2[nH]c3ccc(Cl)cc3c2CCN1CCCc1ccccc1] | LCMS (+APCI) 381 (M⁺) |
| 110 | 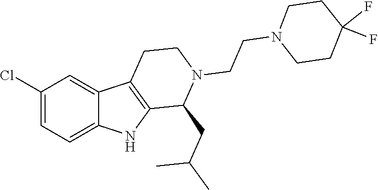 [SMILES: CC(C)C[C@H]1c2[nH]c3ccc(Cl)cc3c2CCN1CCN1CCC(F)(F)CC1] | ¹H NMR (600 MHz, CDCl₃) δ 7.63 (bs, 1H), 7.43 (d, J = 1.8 Hz, 1H), 7.21 (d, J = 8.5 Hz, 1H), 7.09 (dd, J = 8.4, 1.9 Hz, 1H), 3.75 (m, 1H), 3.20 (m, 1H), 2.99 (m, 1H), 2.82 (m, 1H), 2.72 (m, 2H), 2.67 (m, 6H), 2.48 (m, 1H), 1.96 (m, 5H), 1.74 (m, 1H), 1.43 (m, 1H), 1.01 (d, J = 6.6 Hz, 3H), 0.99 (d, J = 6.6 Hz, 3H); LCMS (+ESI) 410 (M⁺). |

Compounds of the present invention can be synthesized using the following Schemes 1-10 in conjunction with knowledge available in the art:

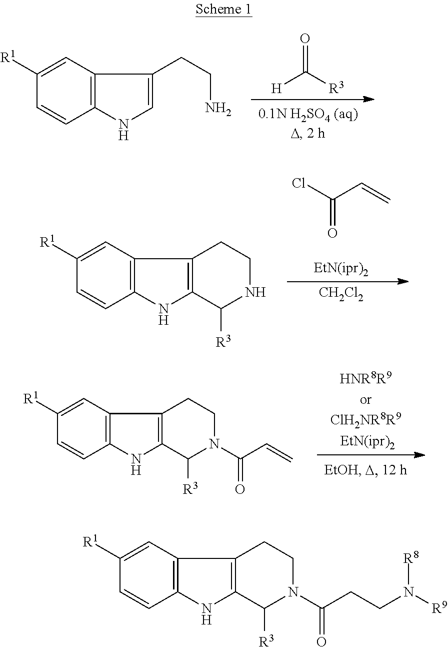

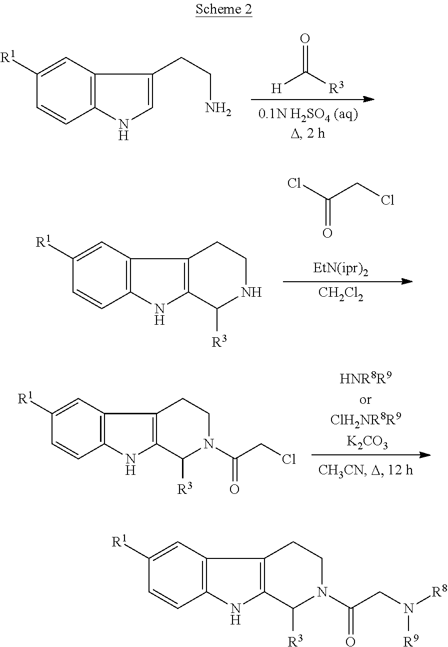

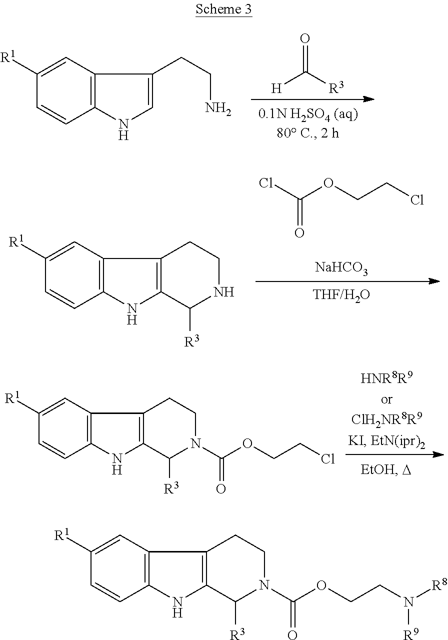

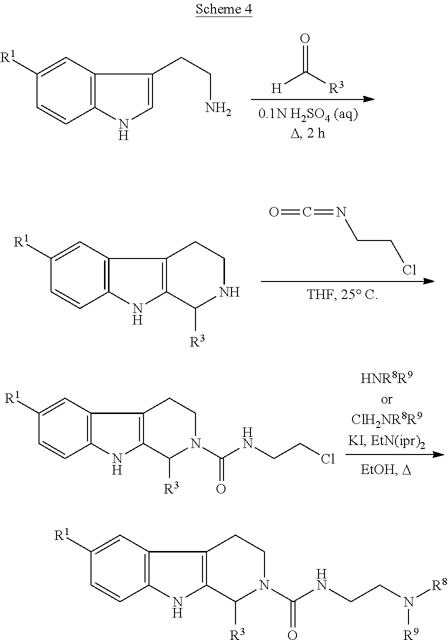

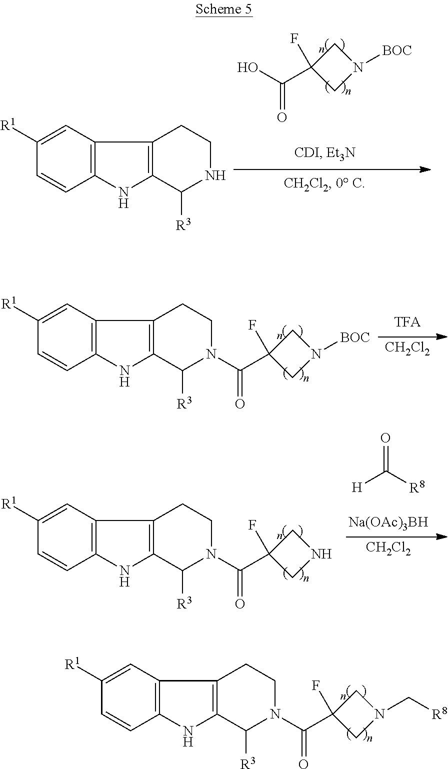

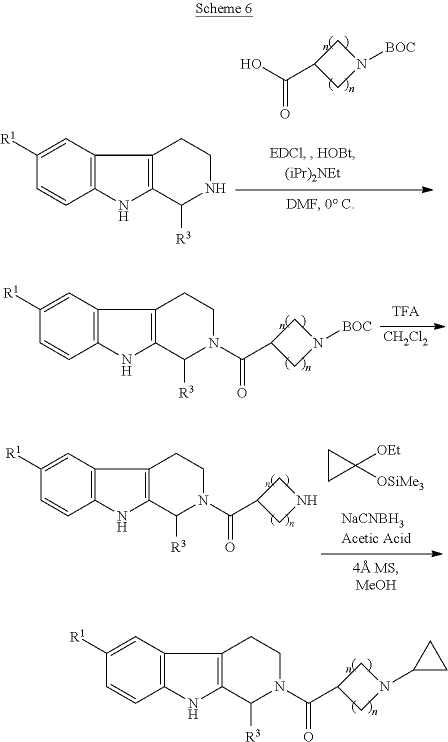

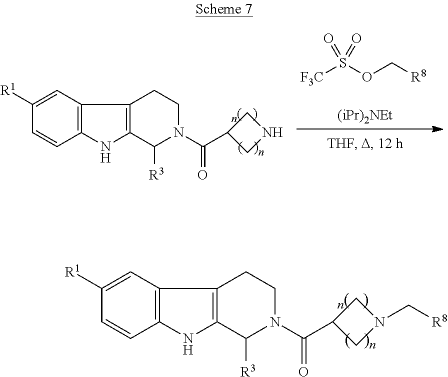

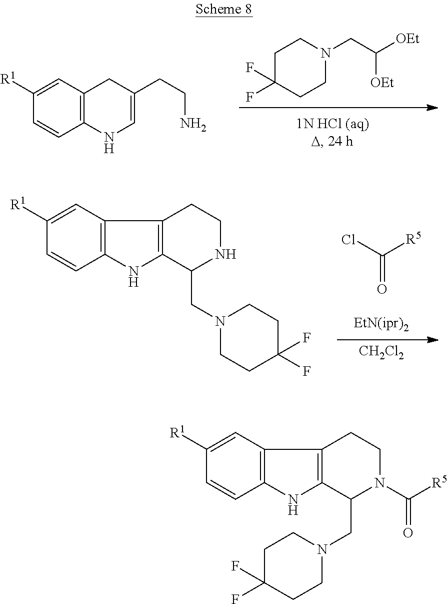

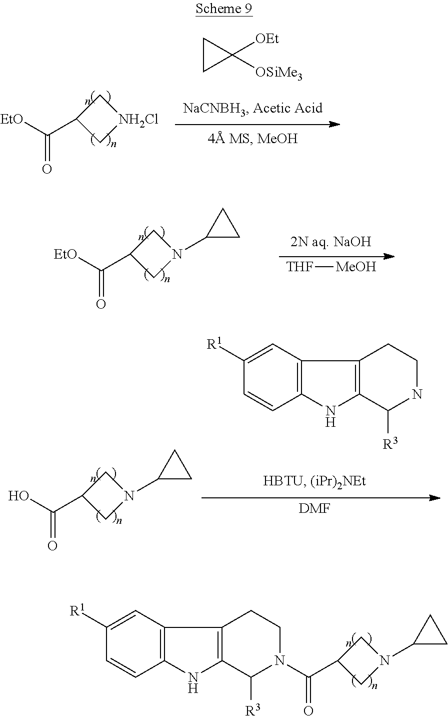

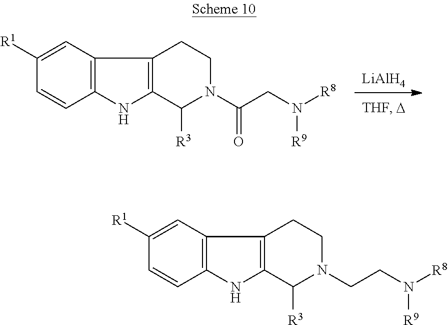

The invention is further illustrated by the following compounds and their preparation.

### Preparation of (S)-1-(6-chloro-1-isobutyl-3,4-dihydro-1H-pyrido[3,4-b]indol-2(9H)-yl)-3-(4,4-difluoropiperidin-1-yl)propan-1-one (Compound 6)

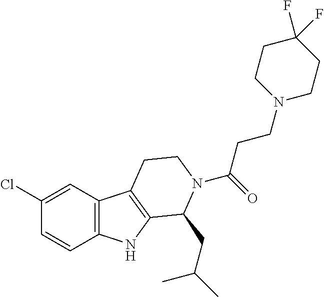 [SMILES: CC(C)C[C@H]1c2[nH]c3ccc(Cl)cc3c2CCN1C(=O)CCN1CCC(F)(F)CC1]

To a 1 L round-bottom flask equipped with a mechanical stirrer, temperature probe, and reflux condenser was added 5-chlorotryptamine hydrochloride (30 g, 130 mmol), 0.1 N sulfuric acid (450 mL), and isovaleraldehyde (21 mL, 195 mmol). The suspension was heated to 80° C. and became a pink homogenous solution. The mixture was stirred for 2 h at 80° C. then allowed to cool to ambient temperature. The solids were collected, washed with MTBE (2×50 mL), and dried overnight under vacuum at 45° C. to give 6-chloro-1-isobutyl-2,3,4,9-tetrahydro-1H-pyrido[3,4-b]indole hydrochloride as an off-white solid 36.5 g (94%, 96.5% AUC by HPLC). Preparative chiral resolution of 6-chloro-1-isobutyl-2,3,4,9-tetrahydro-1H-pyrido[3,4-b]indole hydrochloride (11.9 g) on a Chiralpak IA column (EtOH w/0.1% DEA, 40° C.) gave (S)-6-chloro-1-isobutyl-2,3,4,9-tetrahydro-1H-pyrido[3,4-b]indole (5.4 g, 99.6% ee) and (R)-6-chloro-1-isobutyl-2,3,4,9-tetrahydro-1H-pyrido[3,4-b]indole (5.3, 99.1% ee) as white solids: ¹H NMR (400 MHz, DMSO-d₆) δ 10.83 (s, 1H), 7.35 (d, J=2.0 Hz, 1H), 7.26 (d, J=8.50 Hz, 1H), 6.98 (dd, J=2.1, 8.5 Hz, 1H), 3.94 (dd, J=1.3, 8.9 Hz, 1H), 3.10 (td, J=4.8, 12.7 Hz, 1H), 2.82 (ddd, J=5.6, 7.1, 12.8 Hz, 1H), 2.51-2.57 (m, 2H), 2.16 (br. s, 1H), 1.89-2.01 (m, 1H), 1.66 (ddd, J=3.3, 9.9, 13.4 Hz, 1H), 1.43-1.55 (m, 1H), 0.97 (d, J=6.5 Hz, 3H), 0.92 (d, J=6.7 Hz, 3H); MS (+ESI) 263 (M⁺).

To a stirred solution of (S)-6-chloro-1-isobutyl-2,3,4,9-tetrahydro-1H-pyrido[3,4-b]indole (5 g, 16.7 mmol) in dichloromethane (100 mL) was added N,N-diisopropylethylamine (7.5 mL, 41.8 mmol). The slurry was cooled to 0° C. and acryloyl chloride (1.4 mL, 17.5 mmol) was added at a rate to keep the internal temperature below 10° C. The reaction was stirred for 30 min at 0-10° C., allowed to warm to ambient temperature and stirred overnight. The resulting orange solution was diluted with water (100 mL), and the aqueous phase was extracted with dichloromethane (2×100 mL). The combined organics were washed with brine (100 mL), dried (MgSO₄), filtered over Celite, and concentrated in vacuo. To the oily concentrate was added MTBE (60 mL). The resulting suspension was stirred for 30 min, filtered, and washed with cold MTBE (20 mL). The solids were collected and dried in vacuo at 50° C. to give (S)-1-(6-chloro-1-isobutyl-3,4-dihydro-1H-pyrido[3,4-b]indol-2(9H)-yl)prop-2-en-1-one (2.9 g, 67%, 98.7% AUC by HPLC) as an off-white solid. ¹H NMR (400 MHz, DMSO-d₆) δ 11.12 (s, 1H), 7.41 (d, J=2.0 Hz, 1H), 7.31 (d, J=8.6 Hz, 1H), 7.03 (dd, J=2.0, 8.6 Hz, 1H), 6.95 (dd, J=10.4, 16.4 Hz, 1H), 6.13 (dd, J=2.3, 16.6 Hz, 1H), 5.75-5.82 (m, 1H), 5.72 (dd, J=2.3, 10.4 Hz, 1H), 4.25 (dd, J=4.9, 14.3 Hz, 1H), 3.37-3.50 (m, 1H), 2.56-2.76 (m, 2H), 1.80 (t, J=10.2 Hz, 1H), 1.55-1.70 (m, 2H), 1.05 (d, J=5.8 Hz, 3H), 0.91 (d, J=6.1 Hz, 3H); LCMS (+APCI) 317 (M⁺).

A stirred solution of (S)-1-(6-chloro-1-isobutyl-3,4-dihydro-1H-pyrido[3,4-b]indol-2(9H)-yl)prop-2-en-1-one (1.0 g, 3.2 mmol) in 30 mL of ethanol was added N,N-diisopropylethylamine (1.7 mL, 9.6 mmol) followed by difluoropiperidine hydrochloride (0.75 g, 4.8 mmol). The reaction was heated under reflux for 72 h. The reaction was allowed to cool to ambient temperature and diluted with EtOAC (25 mL) and water (25 mL). The aqueous phase was extracted with EtOAc (25 mL) and the combined organics were washed with brine (25 mL), dried (MgSO₄), filtered, and concentrated in vacuo. The crude residue was purified by column chromatography (40 g SiO2 gel, 30-60% EtOAc/hexanes) to afford compound 6 (1.2 g, 87%) as a white foam. ¹H NMR (400 MHz, DMSO-d₆) δ 11.02-11.16 (m, 1H), 7.41 (d, J=2.1 Hz, 1H), 7.30 (dd, J=0.3, 8.5 Hz, 1H), 7.03 (dd, J=2.1, 8.5 Hz, 1H), 5.65-5.80 (m, 1H), 4.03-4.19 (m, 1H), 3.34-3.47 (m, 1H), 2.61-2.79 (m, 5H), 2.53 (s, 5H), 1.85-1.99 (m, 4H), 1.70-1.80 (m, 1H), 1.53-1.67 (m, 2H), 1.03 (d, J=6.0 Hz, 3H), 0.90 (d, J=6.3 Hz, 3H); LCMS (+ESI) 437 (M⁺).

### Preparation of (S)-1-(6-chloro-1-isobutyl-3,4-dihydro-1H-pyrido[3,4-b]indol-2(9H)-yl)-3-(4-(trifluoromethyl)piperidin-1-yl)propan-1-one (Compound 20)

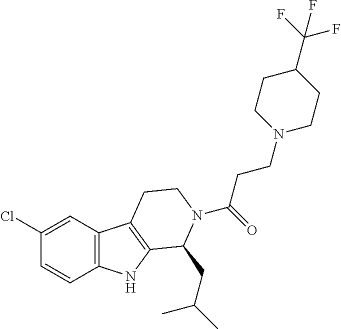 [SMILES: CC(C)C[C@H]1c2[nH]c3ccc(Cl)cc3c2CCN1C(=O)CCN1CCC(C(F)(F)F)CC1]

(S)-1-(6-chloro-1-isobutyl-3,4-dihydro-1H-pyrido[3,4-b]indol-2(9H)-yl)prop-2-en-1-one and 4-(trifluoromethyl)piperidine hydrochloride were combined as previously described for compound 6 to give 20 as a white solid. ¹H NMR (400 MHz, CD₂CL₂) δ 8.02 (s, 1H), 7.45 (d, J=2.0 Hz, 1H), 7.29 (dd, J=0.4, 8.6 Hz, 1H), 7.12 (dd, J=2.1, 8.6 Hz, 1H), 5.89 (dd, J=4.4, 10.0 Hz, 1H), 4.09 (dd, J=4.8, 14.3 Hz, 1H), 3.51 (ddd, J=4.6, 11.8, 14.3 Hz, 1H), 3.04 (t, J=10.0 Hz, 2H), 2.52-2.90 (m, 6H), 2.04 (t, J=11.4 Hz, 2H), 1.68-1.92 (m, 4H), 1.59 (s, 4H), 1.10 (d, J=6.4 Hz, 3H), 1.01 (d, J=6.6 Hz, 3H); LCMS (+ESI) 470 (M⁺).

### Preparation of (S)-1-(6-chloro-1-isobutyl-3,4-dihydro-1H-pyrido[3,4-b]indol-2(9H)-yl)-3-(4-fluoropiperidin-1-yl)propan-1-one (Compound 28)

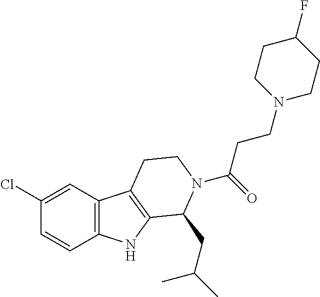 [SMILES: CC(C)C[C@H]1c2[nH]c3ccc(Cl)cc3c2CCN1C(=O)CCN1CCC(F)CC1]

(S)-1-(6-chloro-1-isobutyl-3,4-dihydro-1H-pyrido[3,4-b]indol-2(9H)-yl)prop-2-en-1-one and 4-fluoropiperidine hydrochloride were combined as previously described for compound 6 to give 28 as a white solid. ¹H NMR (400 MHz, CD₂Cl₂) δ 8.02 (s, 1H), 7.38 (d, J=2.1 Hz, 1H), 7.23 (dd, J=0.4, 8.6 Hz, 1H), 7.06 (dd, J=2.0, 8.6 Hz, 1H), 5.84 (dd, J=4.4, 9.9 Hz, 1H), 4.51-4.72 (m, 1H), 3.99-4.08 (m, 1H), 3.45 (ddd, J=4.6, 11.7, 14.3 Hz, 1H), 2.48-2.83 (m, 7H), 2.37 (d, J=3.6 Hz, 2H), 1.49-1.95 (m, 8H), 1.05 (d, J=6.4 Hz, 3H), 0.95 (d, J=6.6 Hz, 3H); LCMS (+ESI) 420 (M⁺).

### Preparation of (S)-1-(6-chloro-1-isobutyl-3,4-dihydro-1H-pyrido[3,4-b]indol-2(9H)-yl)-3-(4-(difluoromethyl)piperidin-1-yl)propan-1-one (Compound 29)

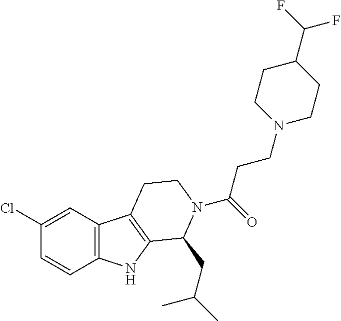 [SMILES: CC(C)C[C@H]1c2[nH]c3ccc(Cl)cc3c2CCN1C(=O)CCN1CCC(C(F)F)CC1]

(S)-1-(6-chloro-1-isobutyl-3,4-dihydro-1H-pyrido[3,4-b]indol-2(9H)-yl)prop-2-en-1-one and 4-(difluoromethyl)piperidine hydrochloride were combined as previously described for compound 6 to give 29 as a white solid. ¹H NMR (600 MHz, CD₂Cl₂-d₂) δ 8.00 (br. s., 1H), 7.41 (d, J=1.9 Hz, 1H), 7.25 (d, J=8.7 Hz, 1H), 7.08 (dd, J=1.98, 8.6 Hz, 1H), 5.86 (dd, J=4.4, 10.1 Hz, 1H), 5.47-5.69 (m, 1H), 4.06 (dd, J=5.1, 14.3 Hz, 1H), 3.47 (ddd, J=4.3, 12.1, 14.3 Hz, 1H), 2.97 (t, J=12.1 Hz, 2H), 2.45-2.84 (m, 6H), 1.97-2.04 (m, 2H), 1.64-1.83 (m, 5H), 1.52-1.60 (m, 1H), 1.38-1.48 (m, 2H), 1.07 (d, J=6.5 Hz, 3H), 0.97 (d, J=6.7 Hz, 3H); LCMS (+APCI) 452 (M⁺).

### Preparation of 1-((S)-6-chloro-1-isobutyl-3,4-dihydro-1H-pyrido[3,4-b]indol-2(9H)-yl)-3-((S)-3-fluoropyrrolidin-1-yl)propan-1-one (Compound 35)

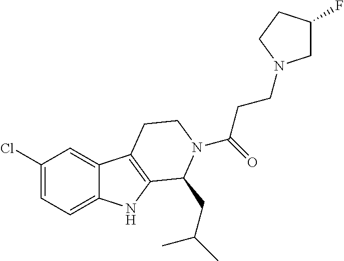 [SMILES: CC(C)C[C@H]1c2[nH]c3ccc(Cl)cc3c2CCN1C(=O)CCN1CC[C@H](F)C1]

(S)-1-(6-chloro-1-isobutyl-3,4-dihydro-1H-pyrido[3,4-b]indol-2(9H)-yl)prop-2-en-1-one and (S)-3-fluoropyrrolidine hydrochloride were combined as previously described for compound 6 to give 35. ¹H NMR (400 MHz, CD₃OD) δ 7.26 (s, 1H), 7.13 (dd, J=0.4, 8.6 Hz, 1H), 6.92 (dd, J=2.0, 8.6 Hz, 1H), 5.74 (dd, J=4.3, 10.0 Hz, 1H), 5.02-5.22 (m, 1H), 4.06 (dd, J=4.8, 14.2 Hz, 1H), 3.21 (ddd, J=4.5, 11.7, 14.2 Hz, 1H), 2.88 (m, 2H), 2.58-2.76 (m, 7H), 2.36 (m, 1H), 2.05 (m, 1H), 1.73 (m, 1H), 1.71 (m, 1H), 1.57 (m, 2H), 1.02 (d, J=6.40 Hz, 3H), 0.86 (d, J=6.5 Hz, 3H); LCMS (+ESI) 406 (M⁺).

### Preparation of (S)-1-(6-chloro-1-isobutyl-3,4-dihydro-1H-pyrido[3,4-b]indol-2(9H)-yl)-3-(4-(2,2-difluoroethyl)piperazin-1-yl)propan-1-one (Compound 73)

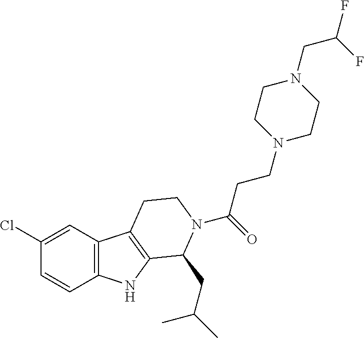 [SMILES: CC(C)C[C@H]1c2[nH]c3ccc(Cl)cc3c2CCN1C(=O)CCN1CCN(CC(F)F)CC1]

(S)-1-(6-chloro-1-isobutyl-3,4-dihydro-1H-pyrido[3,4-b]indol-2(9H)-yl)prop-2-en-1-one and 4-(2,2-difluoroethyl)piperidine hydrochloride were combined as previously described for compound 6 to give 73 as a white solid. ¹H NMR (400 MHz, DMSO-d₆) δ 11.08 (s, 1H), 7.41 (d, J=1.94 Hz, 1H), 7.30 (d, J=8.53 Hz, 1H), 7.02 (dd, J=2.07, 8.53 Hz, 1H), 5.92-6.32 (m, 1H), 5.65-5.80 (m, 1H), 4.01-4.17 (m, 1H), 3.38 (br. s., 1H), 2.62-2.79 (m, 5H), 2.28-2.61 (m, 11H), 1.74 (t, J=10.26 Hz, 1H), 1.52-1.66 (m, 2H), 1.03 (d, J=5.96 Hz, 3H), 0.89 (d, J=6.27 Hz, 3H); LCMS (+ESI) 468 (M⁺).

### Preparation of (S)-1-(6-chloro-1-isobutyl-3,4-dihydro-1H-pyrido[3,4-b]indol-2(9H)-yl)-2-(cyclopropylamino)ethanone (Compound 91)

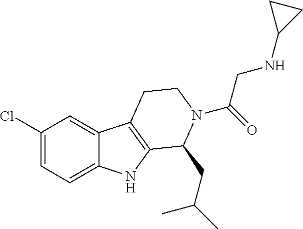 [SMILES: CC(C)C[C@H]1c2[nH]c3ccc(Cl)cc3c2CCN1C(=O)CNC1CC1]

A stirred solution of (S)-6-chloro-1-isobutyl-2,3,4,9-tetrahydro-1H-pyrido[3,4-b]indole (0.5 g, 1.90 mmol) in dichloromethane (50 mL) was cooled to 0° C. in an ice-water bath and treated with N,N-diisopropylethylamine (0.66 mL, 3.80 mmol). Chloroacetyl chloride (0.17 mL, 2.09 mmol) was added dropwise via syringe and the reaction was stirred for 30 min at 0° C. The reaction mixture was allowed to warm to ambient temperature and stirred overnight. The resulting brown solution was diluted with water (50 mL) and the aqueous phase was extracted with dichloromethane (2×50 mL). The combined organics were washed with brine (50 mL), dried (MgSO₄), filtered and concentrated in vacuo. The crude residue was purified by column chromatography (12 g SiO₂ gel, 0-20% EtOAc/hexanes) to afford 2-chloro-1-((S)-6-chloro-1-isobutyl-1,3,4,9)-tetrahydro-beta-carbolin-2-yl)ethanone (0.45 g, 69.7%) as an off-white solid. ¹H NMR (400 MHz, CDCl₃) δ 7.93 (s, 1H), 7.43 (s, 1H), 7.24 (dd, 1H), 7.12 (dd, 1H), 5.80 (m, 1H), 4.21 (m, 2H), 4.06 (m, 1H), 3.61 (m, 1H), 2.77-2.94 (m, 2H), 1.63-1.86 (m, 5H), 1.1 (d, J=6.02 Hz, 3H), 1.01 (d, J=6.34 Hz, 3H); LCMS (+ESI) 340 (M⁺).

To a stirred solution of (S)-2-chloro-1-(6-chloro-lisobutyl-3,4-dihydro-1H-pyrido[3,4-b]indol-2(9H)-ylethanone (0.45 g, 1.15 mmol) in acetonitrile (20 mL) was added dropwise via syringe cycloprpropylamine (0.30 mL, 4.62 mmol). The reaction mixture was allowed to warm to ambient temperature and stirred overnight. The resulting solution was diluted with water (50 mL), and the aqueous phase was extracted with dichloromethane (2×50 mL). The combined organics were washed with brine (50 mL), dried (MgSO₄), filtered and concentrated in vacuo. The crude residue was purified by column chromatography (12 g SiO₂ gel, 0-60% EtOAc/hexanes) to afford compound 91 (0.16 g, 29%) as a white solid. ¹H NMR (400 MHz, DMSO-d₆) δ 11.09 (s, 1H), 7.41 (d, J=2.02 Hz, 1H), 7.30 (dd, J=0.37, 8.56 Hz, 1H), 7.03 (d, J=2.14 Hz, 1H), 5.67-5.73 (m, 1H), 4.03 (dd, J=4.07, 14.09 Hz, 1H), 3.52-3.57 (m, 1H), 3.44-3.63 (m, 1H), 3.44-3.57 (m, 1H), 2.63-2.82 (m, 3H), 2.13 (tt, J=3.42, 6.63 Hz, 1H), 1.72-1.80 (m, 1H), 1.54-1.65 (m, 2H), 1.05 (d, J=5.99 Hz, 3H), 0.90 (d, J=6.24 Hz, 3H), 0.31-0.34 (m, 2H), 0.22-0.26 (m, 2H); HRLCMS m/z 360.1837 (M⁺).

### Preparation of (S)-1-(6-chloro-1-isobutyl-3,4-dihydro-1H-pyrido[3,4-b]indol-2(9H)-yl)-2-((4,4-difluorocyclohexyl)amino)ethanone (Compound 94)

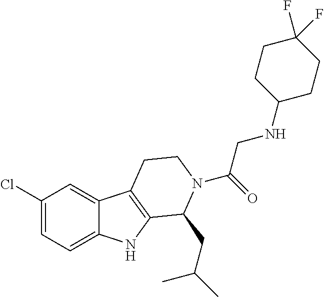 [SMILES: CC(C)C[C@H]1c2[nH]c3ccc(Cl)cc3c2CCN1C(=O)CNC1CCC(F)(F)CC1]

To a stirred solution of 2-chloro-1-((S)-6-chloro-1-isobutyl-1,3,4,9)-tetrahydro-beta-carbolin-2-yl)ethanone (0.25 g, 0.73 mmol) in 10 mL of ethanol was added N,N-diisopropylethylamine (0.64 mL, 3.68 mmol) followed by 4,4-difluorocyclohexanamine hydrochloride (0.19 g, 1.1 mmol) and potassium iodide (0.18 g, 1.1 mmol). The reaction was heated at 100° C. for 12 h. The reaction was allowed to cool to ambient temperature and diluted with EtOAC (25 mL) and water (25 mL). The aqueous phase was extracted with EtOAc (25 mL) and the combined organics were washed with brine (25 mL), dried (MgSO₄), filtered, and concentrated in vacuo. The crude residue was purified by column chromatography to afford compound 94 (0.22 g, 68%) as an off-white powder. ¹H NMR (400 MHz, CD₃OD) δ 7.34 (s, 1H), 7.22 (dd, J=0.4, 8.6 Hz, 1H), 6.99 (dd, J=2.0, 8.6 Hz, 1H), 5.80 (d, J=4.0 Hz, 1H), 4.03-4.08 (m, 1H), 3.68 (d, J=20.1 Hz, 1H), 3.55 (d, J=16.3 Hz, 1H), 3.47 (m, 1H), 2.63-2.82 (m, 3H), 1.68-2.14 (m, 10H), 1.50 (m, 2H), 1.11 (d, J=8.0 Hz, 3H), 0.95 (d, J=8.0 Hz, 3H); LCMS (+ESI) 438 (M⁺).

### Preparation of (S)-1-(6-chloro-1-isobutyl-3,4-dihydro-1H-pyrido[3,4-b]indol-2(9H)-yl)-2-((3,3-difluorocyclobutyl)amino)ethanone (Compound 104)

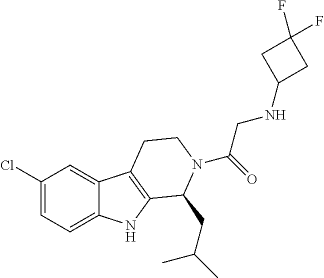 [SMILES: CC(C)C[C@H]1c2[nH]c3ccc(Cl)cc3c2CCN1C(=O)CNC1CC(F)(F)C1]

2-chloro-1-((S)-6-chloro-1-isobutyl-1,3,4,9)-tetrahydro-beta-carbolin-2-yl)ethanone and 1-3,3-difluorocyclobutanamine hydrochloride were combined as previously described for compound 94 to give 104. δ ¹H NMR (400 MHz, CD₃OD) δ 7.38 (s, 1H), 7.25 (dd, J=0.4, 8.6 Hz, 1H), 7.04 (dd, J=2.0, 8.6 Hz, 1H), 5.84 (dd, J=2.0, 8.6 Hz, 1H), 4.04-4.09 (dd, J=4.0, 12.1 Hz, 1H), 3.6 (d, J=16.3 Hz, 1H), 3.56 (d, J=16.3 Hz, 1H), 3.49-3.52 (m, 1H), 3.25-3.30 (m, 1H), 2.72-2.84 (m, 4H), 2.41-2.45 (m, 2H), 1.86 (t, J=8.2 Hz, 1H), 1.67-1.72 (m, 2H), 1.13 (d, J=8.0 Hz, 3H), 0.97 (d, J=8.0 Hz, 3H); LCMS (+ESI) 410 (M⁺).

### Preparation of (S)-1-(6-chloro-1-isobutyl-3,4-dihydro-1H-pyrido[3,4-b]indol-2(9H)-yl)-2-(3,3,3-trifluoropropylamino)ethanone (Compound 108)

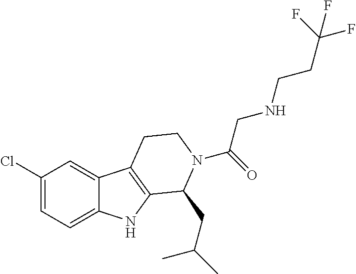 [SMILES: CC(C)C[C@H]1c2[nH]c3ccc(Cl)cc3c2CCN1C(=O)CNCCC(F)(F)F]

2-chloro-1-((S)-6-chloro-1-isobutyl-1,3,4,9)-tetrahydro-beta-carbolin-2-yl)ethanone and 3,3,3-trifluoropropan-1-amine hydrochloride were combined as previously described for compound 94 to give 108. ¹H NMR (400 MHz, CD₃OD) δ 7.25 (d, J=4.1 Hz, 1H), 7.13 (d, J=8.3 Hz, 1H), 6.91 (dd, J=2.0, 8.6 Hz, 1H), 5.73 (dd, J=2.0, 8.6 Hz, 1H), 4.70 (bs, 1H), 3.93-3.98 (dd, J=4.0, 12.1 Hz, 1H), 3.62 (d, J=16.2 Hz, 1H), 3.48 (d, J=16.2 Hz, 1H), 3.37-3.45 (m, 1H), 2.61-2.79 (m, 4H), 2.25-2.36 (m, 2H), 1.75 (t, J=12.0 Hz, 1H), 1.55-1.60 (m, 2H), 1.11 (d, J=8.0 Hz, 3H), 0.95 (d, J=8.0 Hz, 3H); LCMS (+ESI) 416 (M⁺).

### Preparation of 1-(6-chloro-1-isobutyl-3,4-dihydro-1H-pyrido[3,4-b]indol-2(9H)-yl)-2-(3,3-difluoroazetidin-1-yl)ethanone (Compound 85)

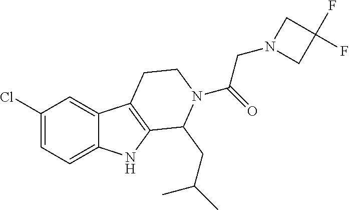 [SMILES: CC(C)CC1c2[nH]c3ccc(Cl)cc3c2CCN1C(=O)CN1CC(F)(F)C1]

2-chloro-1-(6-chloro-1-isobutyl-3,4-dihydro-1H-pyrido[3,4-b]indol-2(9H)-yl)ethanone and 3,3-difluoroazetidine hydrochloride were combined as previously described for compound 94 to give 85. ¹H NMR (400 MHz, CD₃OD) δ 7.25 (s, 1H), 7.14 (d, J=8.2 Hz, 1H), 6.91 (dd, J=2.0, 8.2 Hz, 1H), 5.67 (dd, J=2.0, 8.4 Hz, 1H), 3.91-3.94 (dd, J=4.0, 12.1 Hz, 1H), 3.51-3.70 (m, 4H), 3.37-3.42 (m, 1H), 2.60-2.74 (m, 2H), 1.58 (t, J=8.0 Hz, 1H), 1.52-1.54 (m, 2H), 1.15 (d, J=8.0 Hz, 3H), 0.92 (d, J=8.0 Hz, 3H); LCMS (+ESI) 396 (M⁺).

### Preparation of (6-chloro-1-isobutyl-3,4-dihydro-1H-pyrido[3,4-b]indol-2(9H)-yl)(3-fluoro-1-isobutylazetidin-3-yl)methanone (Compound 103)

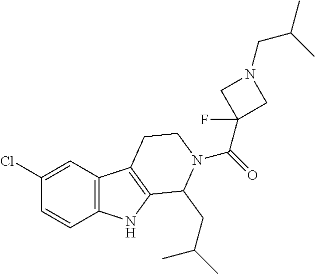 [SMILES: CC(C)CC1c2[nH]c3ccc(Cl)cc3c2CCN1C(=O)C1(F)CN(CC(C)C)C1]

A stirred solution of 3-fluoro-1,3-azetidinedicarboxylic acid tert-butyl ester (1.0 g, 4.56 mmol) in 20 mL of dichloromethane was cooled to 0° C. and treated with 1,1′-carbonyldiimidazole (0.88 g, 5.47 mmol). The reaction mixture was allowed to warm to room temperature and stirred for 3 h. The reaction was treated with triethylamine (1.3 mL, 9.12 mmol) followed by 6-chloro-1-isobutyl-2,3,4,9-tetrahydro-1H-pyrido[3,4-b]indole hydrochloride (1.4 g, 4.56 mmol) then heated to 50° C. and stirred overnight. The resulting solution was diluted with water (50 mL), and the aqueous phase was extracted with ethyl acetate (2×50 mL). The combined organics were washed with brine (50 mL), dried (MgSO₄), filtered and concentrated in vacuo to give 1.2 g (59%) of tert-butyl 3-(6-chloro-1-isobutyl-2,3,4,9-tetrahydro-1H-pyrido[3,4-b]indole-2-carbonyl)-3-fluoroazetidine-1-carboxylate as a solid. ¹H NMR (400 MHz, DMSO-d₆) δ 11.14 (s, 1H), 7.44 (d, J=2.02 Hz, 1H), 7.32 (d, J=8.56 Hz, 1H), 7.04 (dd, J=2.08, 8.56 Hz, 1H), 5.64 (br. s., 1H), 4.34-4.56 (m, 2H), 4.24 (m, 2H), 3.69-3.78 (m, 1H), 3.48-3.58 (m, 1H), 2.73 (d, J=4.59 Hz, 2H), 1.82 (m, 1H), 1.54-1.70 (m, 2H), 1.40 (s, 9H), 1.04 (d, J=6.17 Hz, 3H), 0.92 (d, J=6.36 Hz, 3H). The resulting tert-butyl 3-(6-chloro-1-isobutyl-2,3,4,9-tetrahydro-1H-pyrido[3,4-b] indole-2-carbonyl)-3-fluoroazetidine-1-carboxylate was dissolved in a 3:1 mixture of dichloromethane and trifluoroacetic acid (40 mL) and stirred at room temperature for 4 h. The reaction was concentrated in vacuo and the resulting residue was partitioned between dichloromethane (100 mL) and a saturated solution of bicarbonate (50 mL). The organic layer was washed with brine (50 mL), dried (MgSO₄), filtered and concentrated in vacuo. The crude residue was purified by column chromatography (12 g SiO₂ gel, 4% methanol in dichloromethane) to afford compound 102 0.70 g (66%) as a white solid. ¹H NMR (400 MHz, DMSO-d₆) δ 11.14 (s, 1H), 7.42 (d, J=1.90 Hz, 1H), 7.32 (d, J=8.56 Hz, 1H), 7.04 (dd, J=2.02, 8.56 Hz, 1H), 5.63-5.68 (m, 1H), 3.91-4.12 (m, 2H), 3.65-3.84 (m, 3H), 3.43-3.53 (m, 1H), 2.66-2.80 (m, 3H), 1.76-1.85 (m, 1H), 1.56-1.69 (m, 2H), 0.99-1.09 (m, 3H), 0.91 (s, 3H); LCMS (+ESI) 364 (M⁺).

To a solution of compound 102 (0.20 g, 0.55 mmol) and isobutylaldehyde (0.048 g, 0.66 mmol) in anhydrous dichloromethane (20 mL) was added two drops of acetic acid followed by sodium triacetoxyborohydride (0.18 g, 0.85 mmol). The reaction was stirred at room temperature for 14 h, diluted with aqueous NaHCO₃ (50 mL) and extracted with dichloromethane (2×50 mL). The combined organics were washed with brine, dried (MgSO₄), filtered and concentrated in vacuo. The crude residue was purified by column chromatography (4 g SiO₂ gel, 4% methanol in dichloromethane) to afford 103 (0.09 g, 39%) as a white solid. ¹H NMR (400 MHz, DMSO-d₆) δ 11.13 (s, 1H), 7.42 (d, J=1.90 Hz, 1H), 7.32 (d, J=8.56 Hz, 1H), 7.04 (dd, J=2.05, 8.59 Hz, 1H), 5.65 (dd, J=3.24, 10.39 Hz, 1H), 3.62-3.81 (m, 3H), 3.41-3.55 (m, 3H), 2.63-2.76 (m, 2H), 2.25 (d, J=6.91 Hz, 2H), 1.74-1.86 (m, 1H), 1.45-1.71 (m, 3H), 1.04 (d, J=6.11 Hz, 3H), 0.92 (d, J=6.36 Hz, 3H), 0.83 (d, J=6.60 Hz, 6H); LCMS (+ESI) 420 (M⁺).

### Preparation of (S)-(6-chloro-1-isobutyl-3,4-dihydro-1H-pyrido[3,4-b]indol-2(9H)-yl)(1-(3,3,3-trifluoropropyl)azetidin-3-yl)methanone (Compound 47)

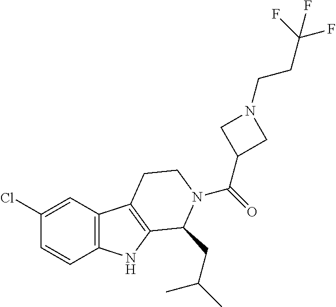 [SMILES: CC(C)C[C@H]1c2[nH]c3ccc(Cl)cc3c2CCN1C(=O)C1CN(CCC(F)(F)F)C1]

To a solution of (S)-6-chloro-1-isobutyl-2,3,4,9-tetrahydro-1H-pyrido[3,4-b]indole (0.54 g, 2.05 mmol) and 1-(tort-butoxycarbonyl)azetidine-3-carboxylic acid (0.45 g, 2.24 mmol) in 10 mL of anhydrous dimethylformamide was added sequentially diisopropylethylatnine (1.42 mL, 8.15 mmol), 1-hydroxybenzotriazole (0.28 g, 2.07 mmol), and 1-ethyl-3-(3-dimethylaminopropyl)carbodiimide (0.54 mL g, 3.08 mmol). The reaction mixture was stirred at room temperature for 4 h and partitioned between dichloromethane (100 mL) and water (100 mL). The organic layer was washed with saturated aqueous NaHCO₃ (50 mL) followed by brine (50 mL), dried (MgSO₄), filtered and concentrated to give a crude oil. The oil was dissolved in a 3:1 mixture of dichloromethane and trifluoroacetic acid (40 mL) and stirred for 4 h at room temperature. The reaction was concentrated in vacuo. The resulting residue was dissolved in dichloromethane (100 mL) and washed with saturated aqueous NaHCO₃ (50 mL). The organic layer was washed with brine (50 mL), dried (MgSO₄), filtered and concentrated in vacuo. The crude material was purified by column chromatography (12 g SiO₂ gel, 4% methanol in dichloromethane) to afford (S)-azetidin-3-yl(6-chloro-1-isobutyl-3,4-dihydro-1H-pyrido[3,4-b]indol-2(9H)-yl)methanone (0.4 g, 76%) as a white foam. LCMS (+ESI) 346 (M⁺).

(S)-azetidin-3-yl(6-chloro-1-isobutyl-3,4-dihydro-1H-pyrido[3,4-b]indol-2(9H)-yl)methanone and 3,33-trifluoropropanal were combined as previously described for compound 103 to give 47 as a white solid. ¹H NMR (400 MHz, DMSO-d₆) δ 11.09 (s, 1H), 7.40 (d, J=1.59 Hz, 1H), 7.30 (d, J=8.50 Hz, 1H), 7.02 (dd, J=1.86, 8.53 Hz, 1H), 5.68 (d, J=8.31 Hz, 1H), 3.79 (dd, J=4.40, 14.24 Hz, 1H), 3.46-3.65 (m, 3H), 3.31-3.44 (m, 2H), 3.13 (t, J=6.08 Hz, 2H), 2.58-2.71 (m, 2H), 2.19-2.33 (m, 2H), 1.67-1.79 (m, 1H), 1.52-1.64 (m, 2H), 1.04 (d, J=5.62 Hz, 3H), 0.90 (d, J=5.81 Hz, 3H); LCMS (+ESI) 442 (M⁺).

### Preparation of (S)-(6-chloro-1-isobutyl-3,4-dihydro-1H-pyrido[3,4-b]indol-2(9H)-yl)(1-cyclopropylazetidin-3-yl)methanone (Compound 92)

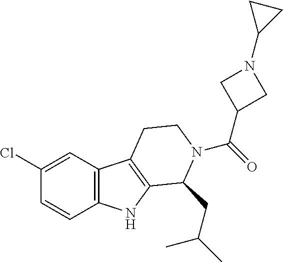 [SMILES: CC(C)C[C@H]1c2[nH]c3ccc(Cl)cc3c2CCN1C(=O)C1CN(C2CC2)C1]

(S)-1-(6-chloro-1-isobutyl-3,4-dihydro-1H-pyrido[3,4-b]indol-2(9H)-yl)prop-2-en-1-one and 1-cyclopropylazetidine-3-carboxylic acid were combined as described for compound 99 to give 92. ¹H NMR (400 MHz, CD₃OD) δ 7.40 (s, 1H), 7.28 (dd, J=0.4, 8.6 Hz, 1H), 7.07 (dd, J=2.0, 8.6 Hz, 1H), 5.84 (d, J=6.0 Hz, 1H), 4.44-4.80 (m, 4H), 4.15-4.41 (m, 1H), 3.60 (ddd, J=4.5, 11.7, 14.2 Hz, 1H), 3.57 (ddd, J=4.5, 11.7, 14.2 Hz, 1H), 3.11 (m, 1H), 2.75-2.78 (m, 2H), 1.71 (m, 1H), 1.57 (m, 2H), 1.68 (d, J=6.4 Hz, 3H), 1.30 (d, J=6.5 Hz, 3H), 1.09-1.14 (m, 4H); LCMS (+ESI) 386 (M⁺).

### Preparation of (S)-(6-chloro-1-isobutyl-3,4-dihydro-1H-pyrido[3,4-b]indol-2(9H)-yl)(1-cyclopropylpiperidin-4-yl)methanone (Compound 93)

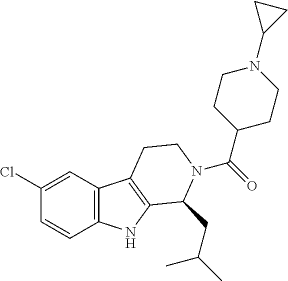 [SMILES: CC(C)C[C@H]1c2[nH]c3ccc(Cl)cc3c2CCN1C(=O)C1CCN(C2CC2)CC1]

(S)-6-chloro-1-isobutyl-2,3,4,9-tetrahydro-1H-pyrido[3,4-b]indole and 1-(tert-butoxycarbonyl)piperidine-4-carboxylic acid were combined as previously described for the synthesis of compound 47 to give (S)-(6-chloro-1-isobutyl-3,4-dihydro-1H-pyrido[3,4-b]indol-2(9H)-yl)(piperidin-4-yl)methanone (1.0 g, 54%) as a white solid. LCMS (+ESI) 374 (M⁺).

A solution of (S)-(6-chloro-1-isobutyl-3,4-dihydro-1H-pyrido[3,4-b]indol-2(9H)-yl)(piperidin-4-yl)methanone (0.90 g, 2.41 mmol) in 5 mL of MeOH was stirred over 4 Å molecular sieves (0.5 g) and treated sequentially with acetic acid (2.5 mL), (1-ethoxycyclopropoxy)trimethylsilane (2.12 g, 12.1 mmol), and sodium cyanoborohydride (0.71 g, 11.2 mmol). The reaction mixture was heated to 50° C. and stirred for 18 h. The reaction was diluted with dichloromethane (20 mL), washed with 1 N aqueous NaOH and brine, dried (MgSO₄), filtered and concentrated in vacuo. The crude residue was purified by column chromatography (12 g SiO₂ gel, 4% methanol in CH₂Cl₂) to afford compound 93 (0.15 g, 15%) as a white solid. ¹H NMR (400 MHz, DMSO-d₆) δ 11.08 (s, 1H), 7.41 (d, J=1.96 Hz, 1H), 7.29 (d, J=8.56 Hz, 1H), 7.27-7.31 (m, 1H), 7.02 (dd, J=2.02, 8.56 Hz, 1H), 5.71-5.76 (m, 1H), 4.15 (dd, J=3.64, 14.21 Hz, 1H), 3.36-3.45 (m, 1H), 2.89-2.97 (m, 2H), 2.65-2.74 (m, 3H), 2.18-2.30 (m, 2H), 1.63-1.77 (m, 3H), 1.50-1.61 (m, 5H), 1.00 (d, J=5.93 Hz, 3H), 0.89 (d, J=6.17 Hz, 3H), 0.36-0.42 (m, 2H), 0.27 (d, J=2.57 Hz, 2H); LCMS (+ESI) 414 (M⁺).

### Preparation of (S)-1-(6-chloro-1-isobutyl-3,4-dihydro-1H-pyrido[3,4-b]indol-2(9H)-yl)-3-cyclohexylpropan-1-one (Compound 43)

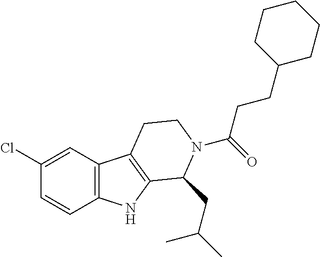 [SMILES: CC(C)C[C@H]1c2[nH]c3ccc(Cl)cc3c2CCN1C(=O)CCC1CCCCC1]

A stirred solution of (S)-6-chloro-1-isobutyl-2,3,4,9-tetrahydro-1H-pyrido[3,4-b]indole (0.1 g, 0.38 mmol) in 20 mL dichloromethane was cooled to 0° C. in an ice-water bath and treated with N,N-diisopropylethylamine (0.13 mL, 0.75 mmol) followed by dropwise addition via syringe of 3-cyclohexylpropanoyl chloride (0.13 g, 0.42 mmol). The reaction was stirred for 30 min at 0° C. The cooling bath was removed and the reaction was stirred overnight. The brown solution was diluted with water (50 mL) and the aqueous phase was extracted with dichloromethane (2×50 mL). The combined organics were washed with brine (50 mL), dried (MgSO₄), filtered and concentrated in vacuo. The crude residue was purified by column chromatography (4 g SiO₂ gel, 20-80% EtOAc/hexanes) to give compound 43 (0.095 g, 63%) as a white solid. ¹H NMR (400 MHz, CD₃OD) δ 7.38 (d, J=1.76 Hz, 1H), 7.25 (d, J=8.60 Hz, 1H), 7.03 (dd, J=1.98, 8.63 Hz, 1H), 5.83-5.88 (m, 1H), 4.13 (br. s., 1H), 3.50-3.59 (m, 1H), 2.75-2.82 (m, 2H), 2.78 (d, J=4.52 Hz, 1H), 2.57-2.67 (m, 1H), 1.52-1.88 (m, 10H), 0.92-139 (m, 12H); LCMS (+ESI) 401 (M⁺).

### Preparation of 1-(6-chloro-1-1((4,4-difluoropiperidin-1-yl)methyl)-3,4-dihydro-1H-pyrido[3,4-b]indol-2(9H)-yl)-3-cyclohexylpropan-1-one (Compound 78)

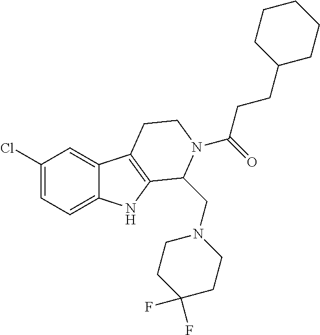 [SMILES: O=C(CCC1CCCCC1)N1CCc2c([nH]c3ccc(Cl)cc23)C1CN1CCC(F)(F)CC1]

A stirred solution of 4,4-difluoropiperidine hydrochloride (0.53 g, 3.36 mmol) and N,N-diisopropylethyamine (1.2 mL, 6.70 mmol) in 5 mL of acetonitrile was treated with 2-bromo-1,1-diethoxyethane (0.25 mL, 1.7 mmol). The reaction mixture was heated at 100° C. for 24 h. The reaction was allowed to cool to ambient temperature and diluted with aqueous NaHCO₃ (100 mL). The aqueous phase was extracted with dichloromethane (2×50 mL) and the combined organic were dried (MgSO4), filtered and concentrated in vacuo to give 1-(2,2-diethoxyethyl)-4,4-difluoropiperidine (0.41 g, 100%) as an amber oil. LCMS (+ESI) 238 (M⁺).

To a sealed tube was added 6-chloro-1-isobutyl-2,3,4,9-tetrahydro-1H-pyrido[3,4-b]indole hydrochloride (0.20 g, 0.87 mmol), 1-(2,2-diethoxyethyl)-4,4-difluoropiperidine (0.21 g, 0.87 mmol), and aqueous 1 N hydrochloric acid (1.7 mL, 1.7 mmol). The mixture was degassed with N₂ and heated at 110° C. for 6 h. The resulting brown solution was allowed to cool to ambient temperature and diluted with aqueous NaHCO₃, extracted with dichloromethane (2×50 mL), dried (MgSO₄), filtered and concentrated to give 90 mg (30%) of crude 6-chloro-1-((4,4-difluoropiperidin-1-yl)methyl)-2,3,4,9-tetrahydro-1H-pyrido[3,4-b]indole by LCMS (+ESI) 340 (M⁺); major impurity (+ESI) 237 (M⁺).

Crude 6-chloro-1-((4,4-difluoropiperidin-1-yl)methyl)-2,3,4,9-tetrahydro-1H-pyrido[3,4-b]indol and 3-cyclohexylpropanoyl chloride were combined as previously described for compound 43 to give 78 as a white solid. ¹H NMR (400 MHz, CDCl₃) δ 8.91 (s, 1H), 7.44 (s, 1H), 7.27 and 7.12 (d, J=8.0 Hz, 2H), 5.59 (dd, J=2.0, 8.1 Hz, 1H), 4.13 (dt, J=1.2, 9.1 Hz, 1H), 3.24-3.33 (m, 1H), 2.90-2.99 (m, 2H), 2.59-2.80 (m, 4H), 2.41-2.49 (m, 2H), 2.00-2.14 (m, 4H), 1.55-1.79 (m, 8H), 1.10-1.36 (m, 3H), and 0.88-1.00 (m, 2H); LCMS (+ESI) 478 (M⁺).

### Preparation of (S)-6-chloro-2-(2-(4,4-difluoropiperidin-1-yl)ethyl)-1-isobutyl-2,3,4,9-tetrahydro-1H-pyrido[3,4-b]indole (Compound 110)

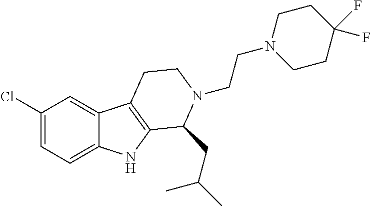 [SMILES: CC(C)C[C@H]1c2[nH]c3ccc(Cl)cc3c2CCN1CCN1CCC(F)(F)CC1]

A stirred solution of (S)-2-chloro-1-(6-chloro-1-isobutyl-3,4-dihydro-1H-pyrido[3,4-b]indol-2(9H)-yl)ethanone (0.61 g, 1.80 mmol) in 50 mL of anhydrous tetrahydrofuran was treated with 4,4-difluoropiperazine hydrochloride (0.43 g, 2.70 mmol), diisopropylethylamine (0.70 g, 5.40 mmol) and sodium iodide (cat.) and heated under reflux for 2 d. The reaction was allowed to cool to ambient temperature and concentrated in vacuo. The resulting residue was treated with aqueous NaHCO₃ (50 mL) and extracted with ethyl acetate (2×50 mL). The combined organics were dried (MgSO₄), filtered, and concentrated. The crude material was purified by column chromatography (40 g SiO₂ gel, 5-60% EtOAc/hexanes) to give (S)-1-(6-chloro-1-isobutyl-3,4-dihydro-1H-pyrido[3,4-b]indol-2(9H)-yl)-2-(4,4-difluoropiperidin-1-yl)ethanone 0.57 g (75%) as a faint yellow oil. ¹H NMR (400 MHz, CDCl₃) δ 7.90 (bs, 1H), 7.42 (d, J=2.0 Hz, 1H), 7.24 (d, J=8.6 Hz, 1H), 7.12 (dd, J=8.5, 2.0 Hz, 1H), 5.82 (m, 1H), 4.13 (m, 1H), 3.49 (m, 1H), 3.34 (d, 2H), 2.89-2.55 (m, 6H), 1.82-1.65 (m, 2H), 1.07 (d, J=6.3 Hz, 3H), 0.99 (d, J=6.5 Hz, 3H); LCMS (+ESI) 424 (M⁺).

To a stirred solution of (S)-1-(6-chloro-1-isobutyl-3,4-dihydro-1H-pyrido[3,4-b]indol-2(9H)-yl)-2-(4,4-difluoropiperidin-1-yl)ethanone (0.57 g, 1.34 mmol) in 30 mL of anhydrous tetrahydrofuran under nitrogen was added via syringe 2N lithium aluminum hydride in tetrahydrofuran (4 mL, 8.0 mmol). The reaction mixture was heated at 55° C. for 2 h. The heating bath was removed, the reaction was allowed to cool to ambient temperature and was stirred 12 h. The reaction was quenched dropwise with 2N aqueous NaOH (1.5 mL) and stirred for 2 h. The mixture was filtered through a plug of Celite. The filtrate was diluted with aqueous NaHCO₃ (50 mL) and extracted with ethyl acetate (2×50 mL). The combined organics were dried Na₂SO₄, filtered, and concentrated in vacuo. The crude material was purified by column chromatography Silica gel chromatography (40 g SiO₂ gel, 10-100% EtOAc/hexanes) to give compound 110 0.25 g (45%) as a light yellow oil. ¹H NMR (600 MHz, CDCl₃) δ 7.63 (bs, 1H), 7.43 (d, J=1.8 Hz, 1H), 7.21 (d, J=8.5 Hz, 1H), 7.09 (dd, J=8.4, 1.9 Hz, 1H), 3.75 (m, 1H), 3.20 (m, 1H), 2.99 (m, 1H), 2.82 (m, 1H), 2.72 (m, 2H), 2.67 (m, 6H), 2.48 (m, 1H), 1.96 (m, 5H), 1.74 (m, 1H), 1.43 (m, 1H), 1.01 (d, J=6.6 Hz, 3H), 0.99 (d, J=6.6 Hz, 3H); LCMS (+ESI) 410 (M⁺).

### Preparation of (S)-(6-bromo-1-isobutyl-3,4-dihydro-1H-pyrido[3,4-b]indol-2(9H)-yl)(1-cyclopropylazetidin-3-yl)methanoneone (Compound 99)

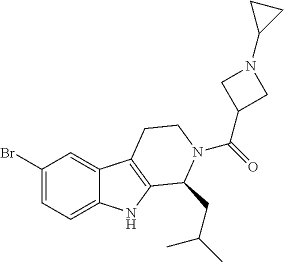 [SMILES: CC(C)C[C@H]1c2[nH]c3ccc(Br)cc3c2CCN1C(=O)C1CN(C2CC2)C1]

A stirred solution of azetidine-3-carboxylic acid ethyl ester hydrochloride (2.0 g, 12.1 mmol) in 100 mL of 1:1 MeOH/EtOH was treated sequentially with [(1-ethoxycyclopropyl)oxy]trimethylsilane (12.6 g, 72.4 mmol), 3 Å molecular sieves (1.0 g), acetic acid (7.26 g, 121 mmol), and sodium cyanoborohydride (3.42 g, 54.5 mmol). The mixture was heated at 75° C. under an inert environment for 20 h. The resulting suspension was filtered and concentrated. The crude residue was diluted in saturated aqueous NaHCO₃ (80 mL) and extracted with ethyl acetate (2×50 mL). The combine organics were dried (MgSO4), filtered, and concentrated in vacuo. The crude material was purification by by column chromatography with an ELSD detector (40 g SiO₂ gel, 0-100% EtOAc/hexanes) to give ethyl 1-cyclopropylazetidine-3-carboxylate 1.25 g (62%) as a colorless oil. ¹H NMR (400 MHz, CDCl₃) δ 4.17 (q, J=7.1 Hz, 2H), 3.70 (m, 2H), 3.48 (m, 2H), 3.43 (p, J=7.8 Hz, 1H), 1.98 (m, 1H), 1.26 (t, J=7.2 Hz, 3H), 0.36-0.40 (m, 4H); LCMS (+ESI) 170 (M⁺).

A stirred solution of ethyl 1-cyclopropylazetidine-3-carboxylate (0.81 g, 4.79 mmol) in 40 mL of 1:1 THF/MeOH was treated with 2N aqueous NaOH (7.20 mL, 14.4 mmol). The reaction mixture was stirred for 8 h at ambient temperature and heated at 80° C. for 1 h. The reaction was allowed to cool to ambient temperature, quenched with a 2N aqueous HCl (7.20 mL, 14.4 mmol) and concentrated to dryness. The residue was taken up in methanol (20 mL), filtered, concentrated and dried at 78° C. under vacuum to give 1-cyclopropylazetidine-3-carboxylic acid as a crude oil 0.66 g (97%). The oil was used in the next reaction without further purification. ¹H NMR (400 MHz, CDCl₃) δ 3.39 (t, J=7.8 Hz, 2H), 3.24 (t, J=7.0 Hz, 2H), 3.10 (m, 1H), 1.82 (m, 1H), 0.31 (m, 2H), 0.16 (m, 2H); LCMS (+ESI) 142 (M⁺).

A stirred suspension of 5-bromotryptamine hydrochloride (17.0 g, 61.7 mmol) in 250 mL of 0.1 N aqueous sulfuric acid was treated with isovaleraldehyde (10.0 mL, 92.6 mmol). The suspension was heated at 80° C. for 3 hr. The resulting solution was allowed to cool to ambient temperature then further cooled to 0° C. in an ice-water bath. The precipitated product was filtered, washed with MTBE (300 mL), dried overnight under high vacuum to give 6-bromo-1-isobutyl-2,3,4,9-tetrahydro-1H-pyrido[3,4-b]indole hydrochloride 19.3 g (92%) as a white solid. ¹H NMR (600 MHz, DMSO-d₆) δ 11.41 (s, 1H), 9.92 (br. s., 1H), 9.46 (br. s., 1H), 7.66 (d, J=1.88 Hz, 1H), 7.33 (d, J=8.56 Hz, 1H), 7.21 (dd, J=1.88, 8.56 Hz, 1H), 4.66 (br. s., 1H), 3.54 (d, J=12.14 Hz, 1H), 3.27 (br. s., 1H), 2.86-3.01 (m, 2H), 2.01-2.09 (m, 1H), 1.91-1.99 (m, 1H), 1.83 (ddd, J=4.09, 10.23, 14.28 Hz, 1H), 1.03 (d, J=6.31 Hz, 3H), 0.97 (d, J=6.59 Hz, 3H). This material was added to 500 mL of saturated aqueous NaHCO₃ and extracted with ethyl acetate (3×100 mL). The ethyl acetate layer was dried (Na₂SO₄), filtered, and concentrated in vacuo to give 6-bromo-1-isobutyl-2,3,4,9-tetrahydro-1H-pyrido[3,4-b]indole 17.0 g (90%) as a white solid. Preparative chiral resolution of 6-bromo-1-isobutyl-2,3,4,9-tetrahydro-1H-pyrido[3,4-b]indole (10.0 g) on a Chiralpak IA column (EtOH w/0.1% DEA, 30° C.) gave (S)-6-bromo-1-isobutyl-2,3,4,9-tetrahydro-1H-pyrido[3,4-b]indole 5.68 g [>100% (high recovery due to excess Et₂NH in sample), >99% ee] and (R)-6-bromo-1-isobutyl-2,3,4,9-tetrahydro-1H-pyrido[3,4-b]indole 4.52 g (90.4%, >99% ee) as white solids.

A stirred solution of (S)-6-bromo-1-isobutyl-2,3,4,9-tetrahydro-1H-pyrido[3,4-b]indole (1.03 g, 2.99 mmol) in 50 mL of anhydrous DMF was treated with 1-cyclopropylazetidine-3-carboxylic acid (0.63 g, 4.49 mmol), N,N-diisopropylethylamine (0.78 mL, 0.58 g, 4.49 mmol) and O-Benzotriazole-N,N,N′,N′-tetramethyl-uronium-hexafluoro-phosphate (HBTU) (1.59 g, 4.19 mmol). The reaction mixture was stirred for 3 h, treated with additional HBTU (0.6 g, 1.8 mmol) and stirred for 1 h. The reaction was diluted with saturated aqueous NaHCO₃ (200 mL) and extracted with ethyl acetate (3×100 mL). The combined organics were washed with 2N aqueous NaOH (50 mL), brine (50 mL), dried (MgSO₄), filtered and concentrated in vacuo. The crude residue was purified by column chromatography (40 g SiO₂ gel, 20:1:0.1 dichloromethane/methanol/ammonium hydroxide) to give compound 990.83 g (61%) as a colorless solid. ¹H NMR (400 MHz, CDCl₃) δ 8.03 (bs, 1H), 7.54 (d, J=1.9 Hz, 1H), 7.24 (dd, J=8.6, 1.8 Hz, 1H), 7.19 (d, J=8.5 Hz, 1H), 5.82 (m, 1H), 3.79 (m, 1H), 3.67 (m, 2H), 3.42-3.52 (m, 4H), 2.69 (m, 2H), 1.85 (m, 1H), 1.75-1.55 (m, 7H), 1.06 (d, J=6.2 Hz, 3H), 0.98 (d, J=6.4 Hz, 3H), 0.36 (m, 4H); LCMS (+ESI) 431 (M⁺).

### Preparation of (S)-(6-bromo-1-isobutyl-3,4-dihydro-1H-pyrido[3,4-b]indol-2(9H)-yl)(1-cyclopropylpiperidin-4-yl)methanone (Compound 97)

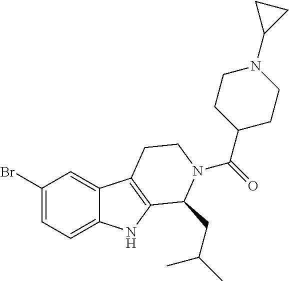 [SMILES: CC(C)C[C@H]1c2[nH]c3ccc(Br)cc3c2CCN1C(=O)C1CCN(C2CC2)CC1]

(S)-6-bromo-1-isobutyl-2,3,4,9-tetrahydro-1H-pyrido[3,4-b]indole and 1-cyclopropylpiperidine-4-carboxylic acid were combined as previously described for compound 99 to give 97. ¹H NMR (400 MHz, CDCl₃) δ 8.19 (bs, 1H), 7.55 (d, J=1.8 Hz, 1H), 7.24 (dd, J=8.6, 1.8 Hz, 1H), 7.17 (d, J=8.5 Hz, 1H), 5.89 (m, 1H), 4.11 (m, 1H), 3.49 (m, 1H), 3.12 (m, 2H), 2.78 (m, 2H), 2.59 (m, 1H), 2.25 (m, 2H), 1.54-1.91 (m, 9H), 0.98 (d, J=6.2 Hz, 3H), 0.92 (d, J=6.4 Hz, 3H), 0.34 (m, 4H); LCMS (+ESI) 458 (M⁺).

### Preparation of (S)-1-(6-bromo-1-isobutyl-3,4-dihydro-1H-pyrido[3,4-b]indol-2(9H)-yl)-2-(cyclopropylamino)ethanone (Compound 98)

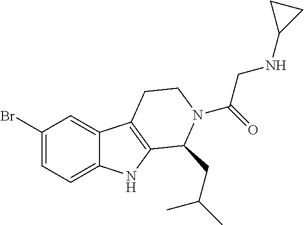 [SMILES: CC(C)C[C@H]1c2[nH]c3ccc(Br)cc3c2CCN1C(=O)CNC1CC1]

(S)-1-(6-bromo-1-isobutyl-3,4-dihydro-1H-pyrido[3,4-b]indol-2(9H)-yl)-2-chloroethanone and cyclopropanamine hydrochloride were combined as previously described for compound 91 to give 98. ¹H NMR (400 MHz, DMSO-d₆) δ 11.10 (s, 1H), 7.55 (d, J=1.59 Hz, 1H), 7.26 (d, J=8.50 Hz, 1H), 7.14 (dd, J=1.90, 8.50 Hz, 1H), 5.76-5.80 (m, 1H), 4.03 (dd, J=3.61, 14.49 Hz, 1H), 3.43-3.63 (m, 2H), 3.31-3.40 (m, 2H), 2.66-2.73 (m, 2H), 2.13 (tt, J=3.29, 6.49 Hz, 1H), 1.72-1.79 (m, 1H), 1.57-1.64 (m, 2H), 1.04 (d, J=5.81 Hz, 3H), 0.90 (d, J=6.05 Hz, 3H), 0.31-0.35 (m, 2H), 0.22-0.26 (m, 2H); LCMS (+ESI) 404 (M⁺).

### Preparation of 1-((S)-6-bromo-1-isobutyl-3,4-dihydro-1H-pyrido[3,4-b]indol-2(9H)-yl)-3-(S)-3-fluoropyrrolidin-1-yl)propan-1-one (Compound 105)

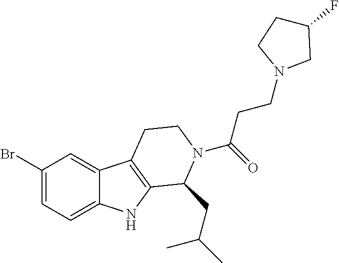 [SMILES: CC(C)C[C@H]1c2[nH]c3ccc(Br)cc3c2CCN1C(=O)CCN1CC[C@H](F)C1]

(S)-6-bromo-1-isobutyl-2,3,4,9-tetrahydro-1H-pyrido[3,4-b]indole and acryloyl chloride were combined as previously described for the synthesis of compound 6 to give (S)-1-(6-bromo-1-isobutyl-3,4-dihydro-1H-pyrido[3,4-b]indol-2(9H)-yl)prop-2-en-1-one. ¹H NMR (400 MHz, CDCl₃): δ 7.99 (br. s., 1H), 7.57 (d, J=1.4 Hz, 1H), 7.22-7.27 (m, 1H), 7.17-7.22 (m, 1H), 6.73 (dd, J=10.6, 16.8 Hz, 1H), 6.35 (dd, J=1.7, 16.7 Hz, 1H), 5.94 (dd, J=4.5, 9.3 Hz, 1H), 5.78 (dd, J=1.7, 10.5 Hz, 1H), 4.19 (dd, J=4.0, 14.2 Hz, 1H), 3.54 (ddd, J=5.0, 11.1, 14.2 Hz, 1H), 3.06-3.20 (m, 1H), 2.62-2.97 (m, 2H), 1.70-1.95 (m, 1H), 1.50-1.70 (m, 2H), 0.94-1.18 (m, 6H); LCMS (+ESI) 361(M⁺).

(S)-1-(6-Bromo-1-isobutyl-3,4-dihydro-1H-pyrido[3,4-b]indol-2(9H)-yl)prop-2-en-1-one and (S)-3-fluoropyrrolidine hydrochloride were combined as previously described for compound 6 to give 105. ¹H NMR (400 MHz, CD₃OD) δ 7.1 (d, J=4.1 Hz, 1H), 7.14-7.24 (m, 2H), 5.86 (dd, J=4.3, 8.5 Hz, 1H), 5.12-5.32 (m, 1H), 4.18 (dd, J=4.3, 14.2 Hz, 1H), 3.56 (ddd, J=4.5, 11.7, 14.2 Hz, 1H), 2.66-3.34 (m, 9H), 2.17-2.48 (m, 1H), 2.47 (m, 1H), 2.17 (m, 1H), 1.85 (t, J=8.6 Hz, 1H), 1.66-1.70 (m, 2H), 1.14 (d, J=6.4 Hz, 3H), 0.97 (d, J=6.5 Hz, 3H); LCMS (+ESI) 450 (M⁺).

### EXAMPLES

The following examples, including the experiments conducted and results achieved are provided for illustrative purposes only and are not to be construed as limiting the invention.

### Example 1

| Ingredients | Concentration (w/v %) |
|---|---|
| Compound of Formula I, II, or III | 0.01-1% |
| Mannitol | 2.0% |
| Sodium Acetate | 0.5% |
| Acetic Acid | 0.02% |
| PEG 8000 | 2.0% |
| Polysorbate 80 | 1.0% |
| HPMC | 0.5% |
| Sodium Hydroxide/Hydrochloric acid | For adjusting pH to 4.5 |
| Purified water | q.s. to 100% |

### Example 2

Compounds of the present invention were tested for potency against Ad5 in an Ad5 genome replication assay, a cell-based QPCR assay of Ad5 genome replication in A549 cells, a human lung carcinoma cell line. Serotype of human Ad5, used in this replication assay can also replicate in a NZW rabbit model of adenoviral ocular keratitis.

EC₅₀ values (μM) for inhibition of adenoviral replication were determined by QPCR. Briefly, 10,000 cultured A549 cells were inoculated with 25,000 pfu/well for 3 hrs in 2% FBS F12-K A549 culture media. Following inoculation, A549 cells were treated with log unit dilutions of compound of Formula I (˜10 μM thru˜10 pM) in 10% FBS F12-K culture media with 0.3% DMSO for an additional 3 days. Cell viability was assessed with Alamar Blue (10 μl/well for 2 hrs; fluorescence Ex353; Em595 nm). Plates were washed 3 times with 125 μl PBS, cell lysates were prepared for QPCR analysis using Cells to SNP kit (Life Technologies). Ad5 genome levels per cell were determined with RNaseP and Ad5 hexon Taqman Assays (Life Technologies). EC₅₀, the concentration that inhibited Ad5 genome replication formation by 50%, was estimated from plots of Ad5 genomes/cell vs. compound concentration.

The results shown in TABLE 2 indicate that the tested compounds of the present invention demonstrated significant antiviral activity in the phenotypic adenoviral assay.

TABLE 2

| Compound | | Ad5 qPCR |
|---|---|---|
| # | Structure | EC₅₀ (μM) |
| 1 | 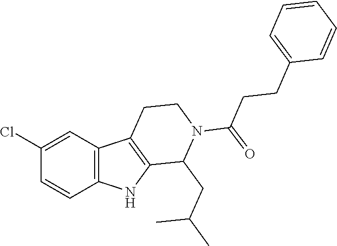 [SMILES: CC(C)CC1c2[nH]c3ccc(Cl)cc3c2CCN1C(=O)CCc1ccccc1] | ++ |
| 2 | 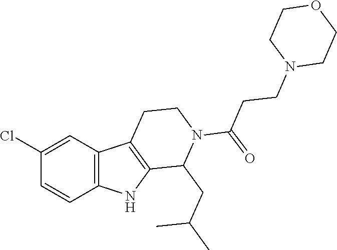 [SMILES: CC(C)CC1c2[nH]c3ccc(Cl)cc3c2CCN1C(=O)CCN1CCOCC1] | + |
| 3 | 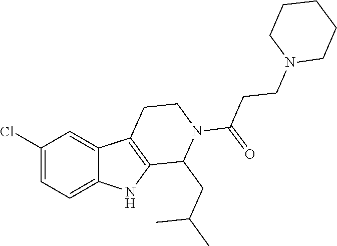 [SMILES: CC(C)CC1c2[nH]c3ccc(Cl)cc3c2CCN1C(=O)CCN1CCCCC1] | + |
| 4 | 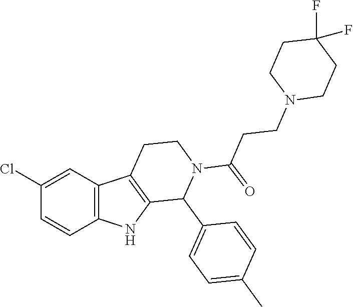 [SMILES: Cc1ccc(C2c3[nH]c4ccc(Cl)cc4c3CCN2C(=O)CCN2CCC(F)(F)CC2)cc1] | ++ |
| 5 | 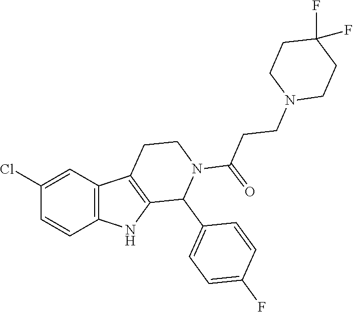 [SMILES: O=C(CCN1CCC(F)(F)CC1)N1CCc2c([nH]c3ccc(Cl)cc23)C1c1ccc(F)cc1] | + |
| 6 | 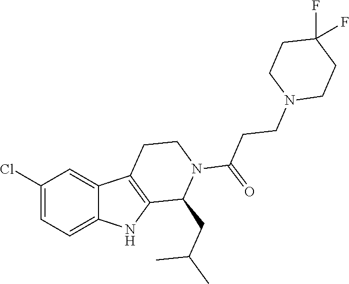 [SMILES: CC(C)C[C@H]1c2[nH]c3ccc(Cl)cc3c2CCN1C(=O)CCN1CCC(F)(F)CC1] | +++ |
| 7 | 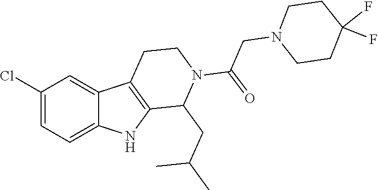 [SMILES: CC(C)CC1c2[nH]c3ccc(Cl)cc3c2CCN1C(=O)CN1CCC(F)(F)CC1] | ++ |
| 8 | 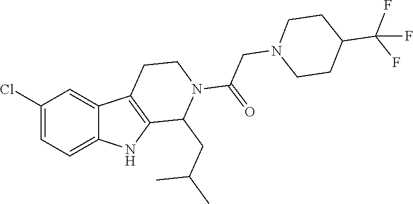 [SMILES: CC(C)CC1c2[nH]c3ccc(Cl)cc3c2CCN1C(=O)CN1CCC(C(F)(F)F)CC1] | ++ |
| 9 | 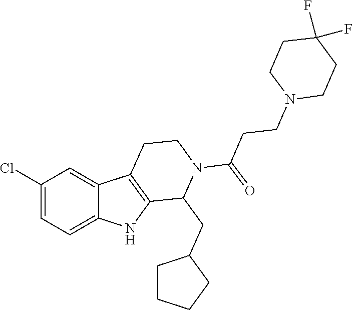 [SMILES: O=C(CCN1CCC(F)(F)CC1)N1CCc2c([nH]c3ccc(Cl)cc23)C1CC1CCCC1] | ++ |
| 10 | 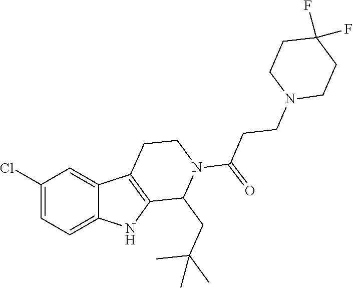 [SMILES: CC(C)(C)CC1c2[nH]c3ccc(Cl)cc3c2CCN1C(=O)CCN1CCC(F)(F)CC1] | ++ |
| 11 | 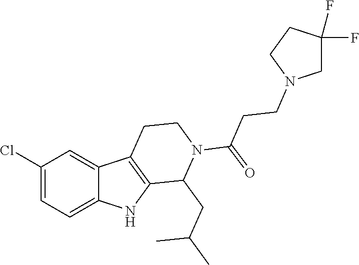 [SMILES: CC(C)CC1c2[nH]c3ccc(Cl)cc3c2CCN1C(=O)CCN1CCC(F)(F)C1] | ++ |
| 12 | 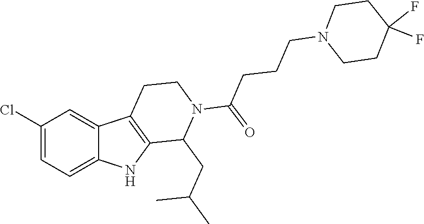 [SMILES: CC(C)CC1c2[nH]c3ccc(Cl)cc3c2CCN1C(=O)CCCN1CCC(F)(F)CC1] | + |
| 13 | 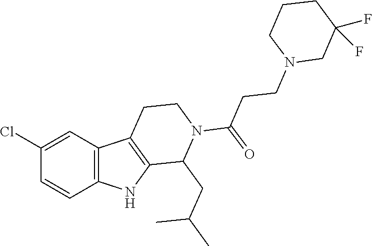 [SMILES: CC(C)CC1c2[nH]c3ccc(Cl)cc3c2CCN1C(=O)CCN1CCCC(F)(F)C1] | ++ |
| 14 | 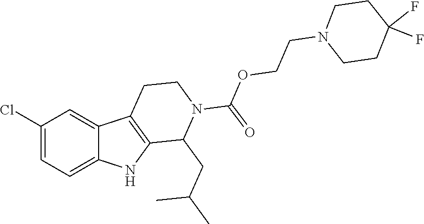 [SMILES: CC(C)CC1c2[nH]c3ccc(Cl)cc3c2CCN1C(=O)OCCN1CCC(F)(F)CC1] | ++ |
| 15 | 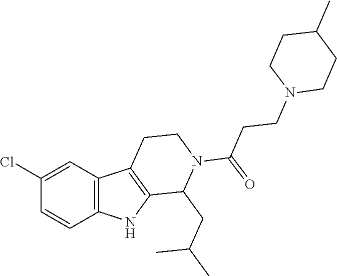 [SMILES: CC(C)CC1c2[nH]c3ccc(Cl)cc3c2CCN1C(=O)CCN1CCC(C)CC1] | + |
| 16 | 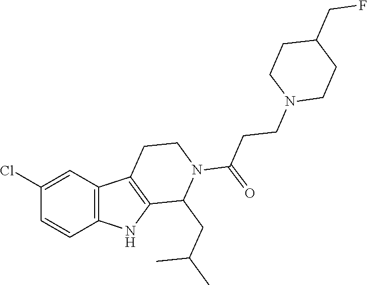 [SMILES: CC(C)CC1c2[nH]c3ccc(Cl)cc3c2CCN1C(=O)CCN1CCC(CF)CC1] | ++ |
| 17 | 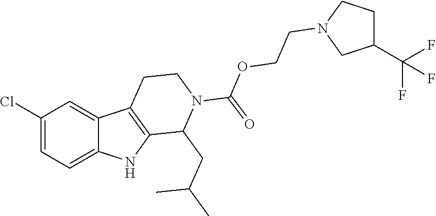 [SMILES: CC(C)CC1c2[nH]c3ccc(Cl)cc3c2CCN1C(=O)OCCN1CCC(C(F)(F)F)C1] | ++ |
| 18 | 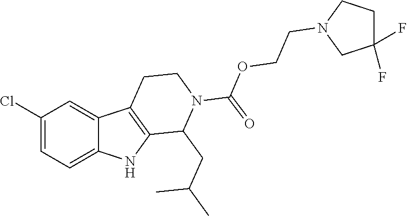 [SMILES: CC(C)CC1c2[nH]c3ccc(Cl)cc3c2CCN1C(=O)OCCN1CCC(F)(F)C1] | ++ |
| 19 | 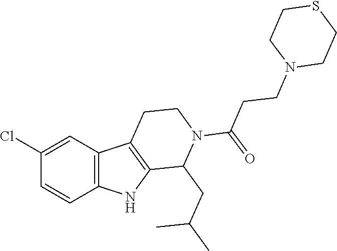 [SMILES: CC(C)CC1c2[nH]c3ccc(Cl)cc3c2CCN1C(=O)CCN1CCSCC1] | ++ |
| 20 | 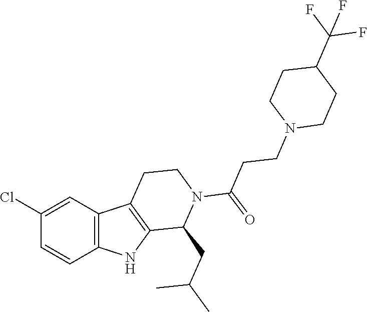 [SMILES: CC(C)C[C@H]1c2[nH]c3ccc(Cl)cc3c2CCN1C(=O)CCN1CCC(C(F)(F)F)CC1] | ++ |
| 21 | 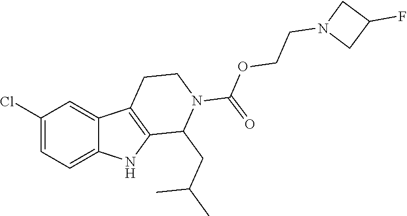 [SMILES: CC(C)CC1c2[nH]c3ccc(Cl)cc3c2CCN1C(=O)OCCN1CC(F)C1] | + |
| 22 | 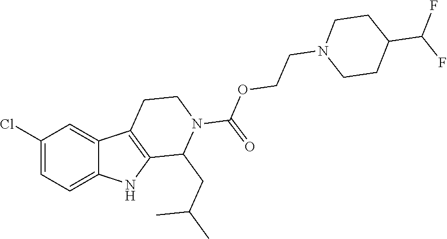 [SMILES: CC(C)CC1c2[nH]c3ccc(Cl)cc3c2CCN1C(=O)OCCN1CCC(C(F)F)CC1] | + |
| 23 | 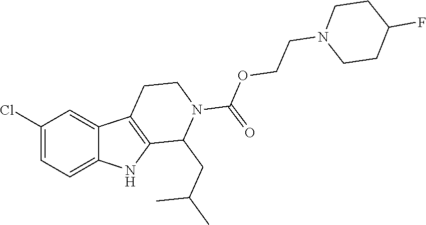 [SMILES: CC(C)CC1c2[nH]c3ccc(Cl)cc3c2CCN1C(=O)OCCN1CCC(F)CC1] | + |
| 24 | 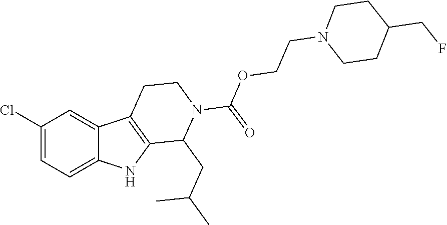 [SMILES: CC(C)CC1c2[nH]c3ccc(Cl)cc3c2CCN1C(=O)OCCN1CCC(CF)CC1] | + |
| 25 | 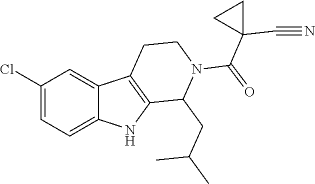 [SMILES: CC(C)CC1c2[nH]c3ccc(Cl)cc3c2CCN1C(=O)C1(C#N)CC1] | + |
| 26 | 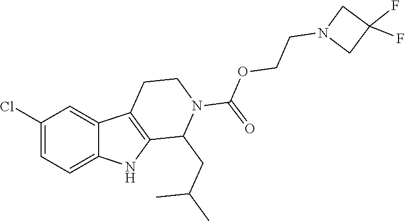 [SMILES: CC(C)CC1c2[nH]c3ccc(Cl)cc3c2CCN1C(=O)OCCN1CC(F)(F)C1] | ++ |
| 27 | 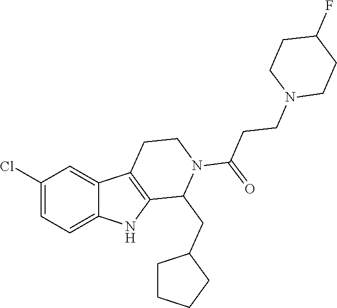 [SMILES: O=C(CCN1CCC(F)CC1)N1CCc2c([nH]c3ccc(Cl)cc23)C1CC1CCCC1] | ++ |
| 28 | 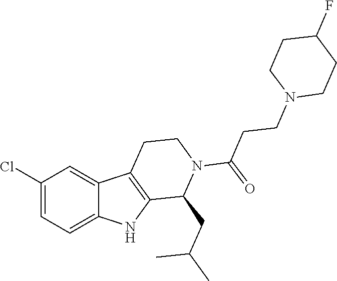 [SMILES: CC(C)C[C@H]1c2[nH]c3ccc(Cl)cc3c2CCN1C(=O)CCN1CCC(F)CC1] | ++ |
| 29 | 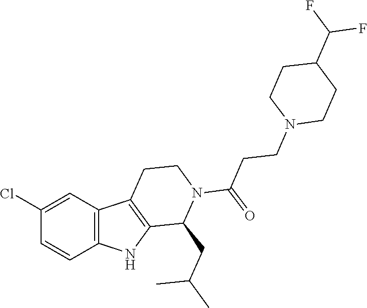 [SMILES: CC(C)C[C@H]1c2[nH]c3ccc(Cl)cc3c2CCN1C(=O)CCN1CCC(C(F)F)CC1] | ++ |
| 30 | 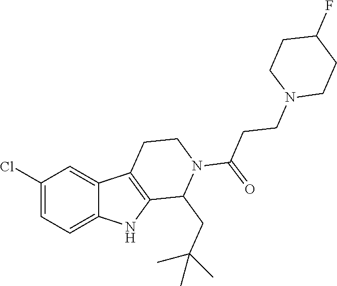 [SMILES: CC(C)(C)CC1c2[nH]c3ccc(Cl)cc3c2CCN1C(=O)CCN1CCC(F)CC1] | + |
| 31 | 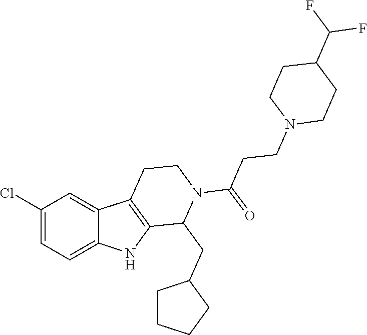 [SMILES: O=C(CCN1CCC(C(F)F)CC1)N1CCc2c([nH]c3ccc(Cl)cc23)C1CC1CCCC1] | ++ |
| 32 | 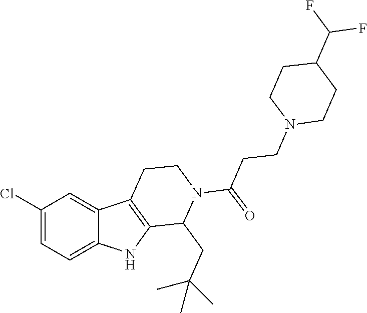 [SMILES: CC(C)(C)CC1c2[nH]c3ccc(Cl)cc3c2CCN1C(=O)CCN1CCC(C(F)F)CC1] | + |
| 33 | 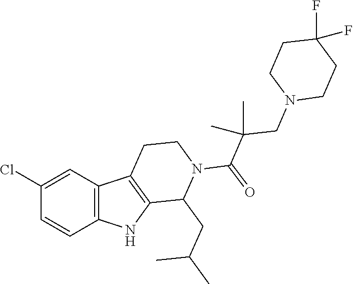 [SMILES: CC(C)CC1c2[nH]c3ccc(Cl)cc3c2CCN1C(=O)C(C)(C)CN1CCC(F)(F)CC1] | + |
| 34 | 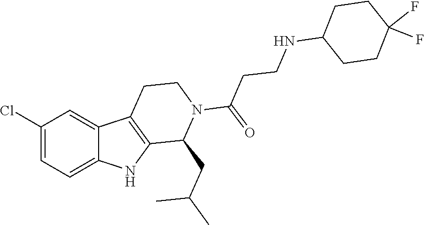 [SMILES: CC(C)C[C@H]1c2[nH]c3ccc(Cl)cc3c2CCN1C(=O)CCNC1CCC(F)(F)CC1] | + |
| 35 | 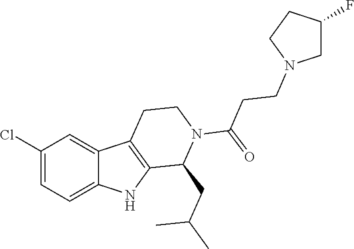 [SMILES: CC(C)C[C@H]1c2[nH]c3ccc(Cl)cc3c2CCN1C(=O)CCN1CC[C@H](F)C1] | ++ |
| 36 | 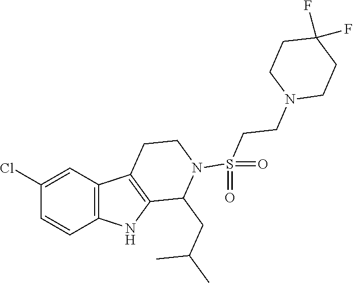 [SMILES: CC(C)CC1c2[nH]c3ccc(Cl)cc3c2CCN1S(=O)(=O)CCN1CCC(F)(F)CC1] | + |
| 37 | 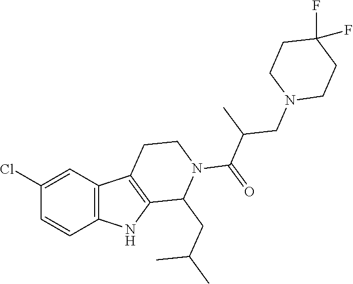 [SMILES: CC(C)CC1c2[nH]c3ccc(Cl)cc3c2CCN1C(=O)C(C)CN1CCC(F)(F)CC1] | ++ |
| 38 | 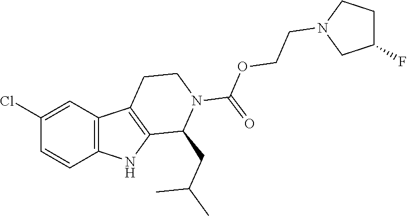 [SMILES: CC(C)C[C@H]1c2[nH]c3ccc(Cl)cc3c2CCN1C(=O)OCCN1CC[C@H](F)C1] | ++ |
| 39 | 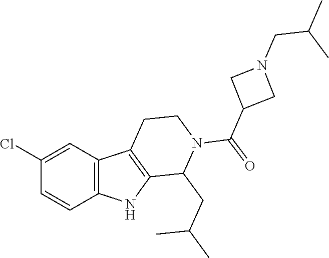 [SMILES: CC(C)CC1c2[nH]c3ccc(Cl)cc3c2CCN1C(=O)C1CN(CC(C)C)C1] | + |
| 40 | 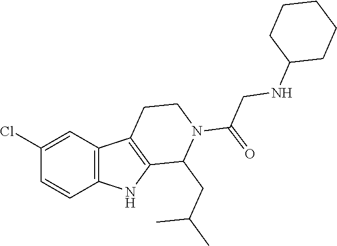 [SMILES: CC(C)CC1c2[nH]c3ccc(Cl)cc3c2CCN1C(=O)CNC1CCCCC1] | + |
| 41 | 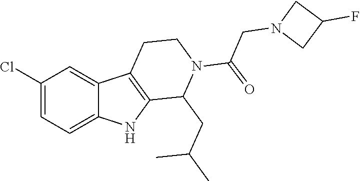 [SMILES: CC(C)CC1c2[nH]c3ccc(Cl)cc3c2CCN1C(=O)CN1CC(F)C1] | ++ |
| 42 | 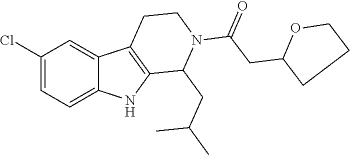 [SMILES: CC(C)CC1c2[nH]c3ccc(Cl)cc3c2CCN1C(=O)CC1CCCO1] | ++ |
| 43 | 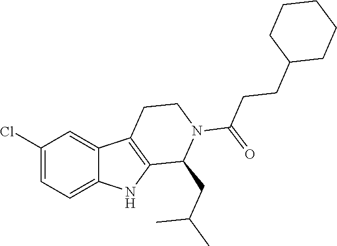 [SMILES: CC(C)C[C@H]1c2[nH]c3ccc(Cl)cc3c2CCN1C(=O)CCC1CCCCC1] | +++ |
| 44 | 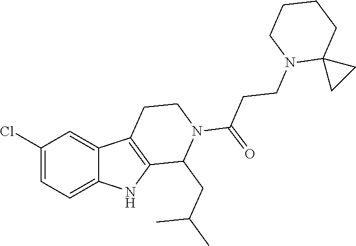 [SMILES: CC(C)CC1c2[nH]c3ccc(Cl)cc3c2CCN1C(=O)CCN1CCCCC12CC2] | ++ |
| 45 | 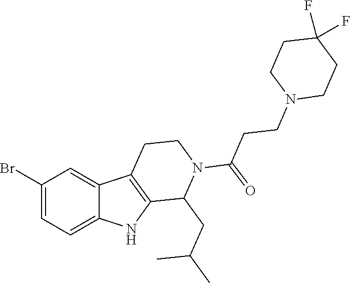 [SMILES: CC(C)CC1c2[nH]c3ccc(Br)cc3c2CCN1C(=O)CCN1CCC(F)(F)CC1] | +++ |
| 46 | 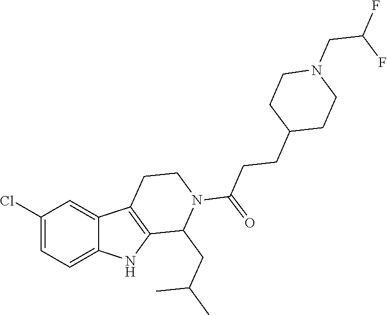 [SMILES: CC(C)CC1c2[nH]c3ccc(Cl)cc3c2CCN1C(=O)CCC1CCN(CC(F)F)CC1] | ++ |
| 47 | 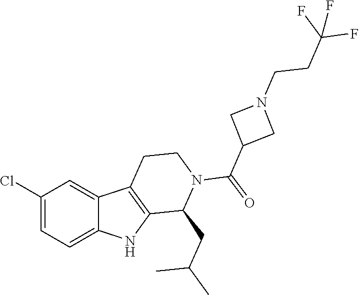 [SMILES: CC(C)C[C@H]1c2[nH]c3ccc(Cl)cc3c2CCN1C(=O)C1CN(CCC(F)(F)F)C1] | ++ |
| 48 | 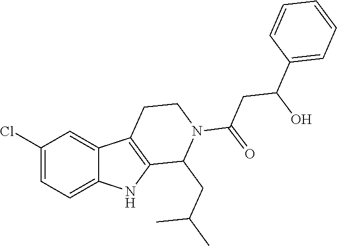 [SMILES: CC(C)CC1c2[nH]c3ccc(Cl)cc3c2CCN1C(=O)CC(O)c1ccccc1] | ++ |
| 49 | 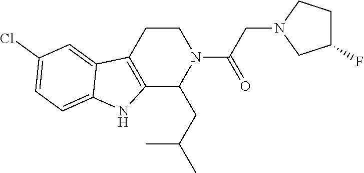 [SMILES: CC(C)CC1c2[nH]c3ccc(Cl)cc3c2CCN1C(=O)CN1CC[C@H](F)C1] | + |
| 50 | 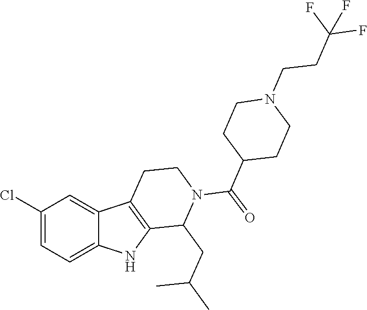 [SMILES: CC(C)CC1c2[nH]c3ccc(Cl)cc3c2CCN1C(=O)C1CCN(CCC(F)(F)F)CC1] | ++ |
| 51 | 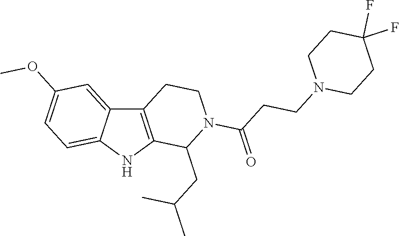 [SMILES: COc1ccc2[nH]c3c(c2c1)CCN(C(=O)CCN1CCC(F)(F)CC1)C3CC(C)C] | ++ |
| 52 | 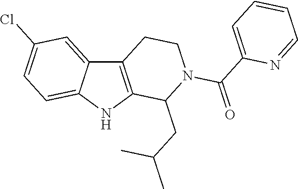 [SMILES: CC(C)CC1c2[nH]c3ccc(Cl)cc3c2CCN1C(=O)c1ccccn1] | ++ |
| 53 | 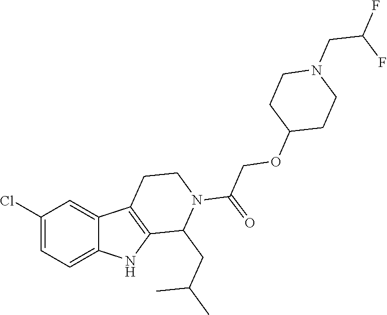 [SMILES: CC(C)CC1c2[nH]c3ccc(Cl)cc3c2CCN1C(=O)COC1CCN(CC(F)F)CC1] | + |
| 54 | 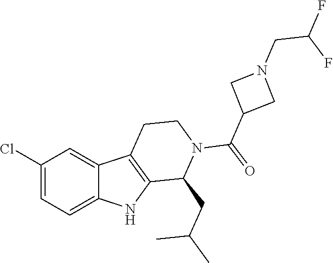 [SMILES: CC(C)C[C@H]1c2[nH]c3ccc(Cl)cc3c2CCN1C(=O)C1CN(CC(F)F)C1] | ++ |
| 55 | 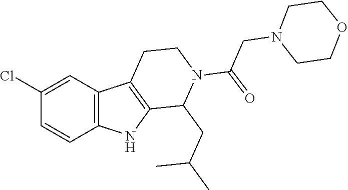 [SMILES: CC(C)CC1c2[nH]c3ccc(Cl)cc3c2CCN1C(=O)CN1CCOCC1] | + |
| 56 | 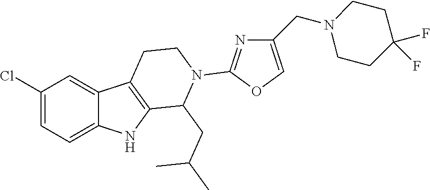 [SMILES: CC(C)CC1c2[nH]c3ccc(Cl)cc3c2CCN1c1nc(CN2CCC(F)(F)CC2)co1] | ++ |
| 57 | 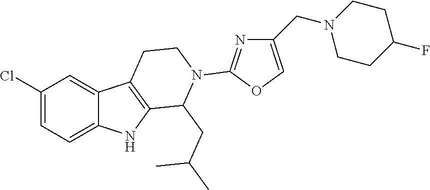 [SMILES: CC(C)CC1c2[nH]c3ccc(Cl)cc3c2CCN1c1nc(CN2CCC(F)CC2)co1] | ++ |
| 58 | 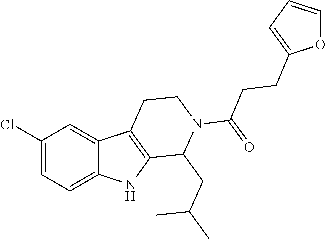 [SMILES: CC(C)CC1c2[nH]c3ccc(Cl)cc3c2CCN1C(=O)CCc1ccco1] | +++ |
| 59 | 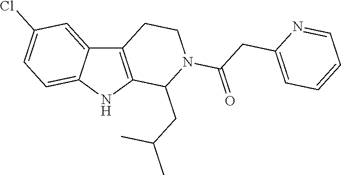 [SMILES: CC(C)CC1c2[nH]c3ccc(Cl)cc3c2CCN1C(=O)Cc1ccccn1] | + |
| 60 | 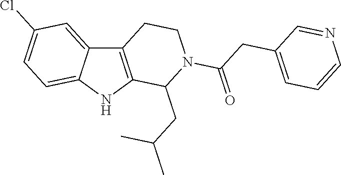 [SMILES: CC(C)CC1c2[nH]c3ccc(Cl)cc3c2CCN1C(=O)Cc1cccnc1] | + |
| 61 | 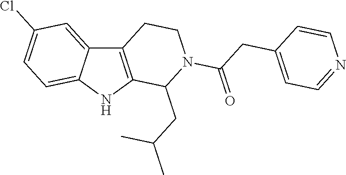 [SMILES: CC(C)CC1c2[nH]c3ccc(Cl)cc3c2CCN1C(=O)Cc1ccncc1] | + |
| 62 | 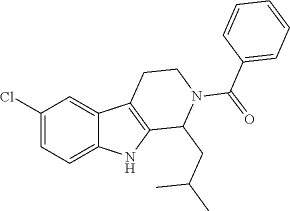 [SMILES: CC(C)CC1c2[nH]c3ccc(Cl)cc3c2CCN1C(=O)c1ccccc1] | ++ |
| 63 | 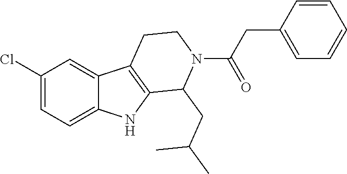 [SMILES: CC(C)CC1c2[nH]c3ccc(Cl)cc3c2CCN1C(=O)Cc1ccccc1] | ++ |
| 64 | 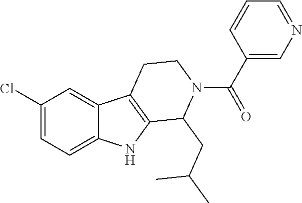 [SMILES: CC(C)CC1c2[nH]c3ccc(Cl)cc3c2CCN1C(=O)c1cccnc1] | ++ |
| 65 | 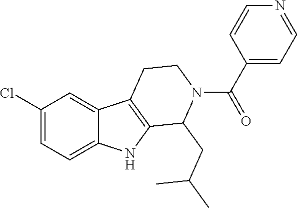 [SMILES: CC(C)CC1c2[nH]c3ccc(Cl)cc3c2CCN1C(=O)c1ccncc1] | ++ |
| 66 | 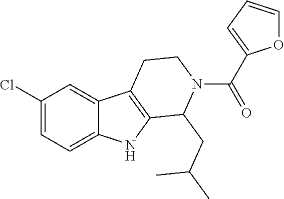 [SMILES: CC(C)CC1c2[nH]c3ccc(Cl)cc3c2CCN1C(=O)c1ccco1] | + |
| 67 | 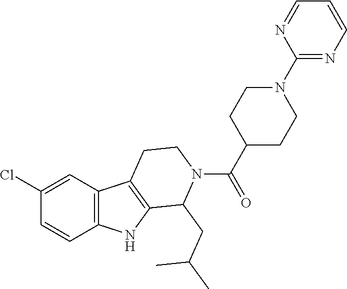 [SMILES: CC(C)CC1c2[nH]c3ccc(Cl)cc3c2CCN1C(=O)C1CCN(c2ncccn2)CC1] | ++ |
| 68 | 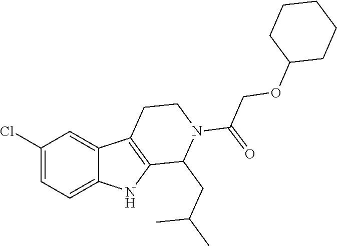 [SMILES: CC(C)CC1c2[nH]c3ccc(Cl)cc3c2CCN1C(=O)COC1CCCCC1] | ++ |
| 69 | 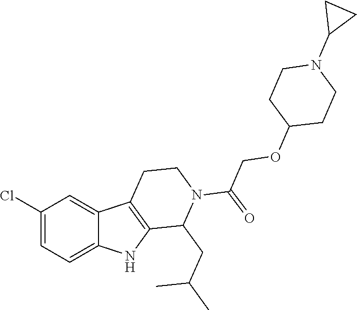 [SMILES: CC(C)CC1c2[nH]c3ccc(Cl)cc3c2CCN1C(=O)COC1CCN(C2CC2)CC1] | + |
| 70 | 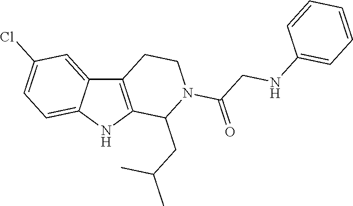 [SMILES: CC(C)CC1c2[nH]c3ccc(Cl)cc3c2CCN1C(=O)CNc1ccccc1] | +++ |
| 71 | 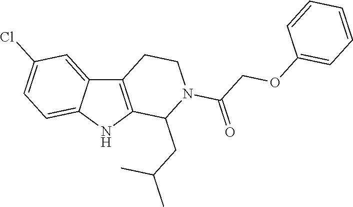 [SMILES: CC(C)CC1c2[nH]c3ccc(Cl)cc3c2CCN1C(=O)COc1ccccc1] | ++ |
| 72 | 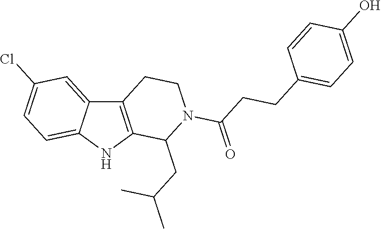 [SMILES: CC(C)CC1c2[nH]c3ccc(Cl)cc3c2CCN1C(=O)CCc1ccc(O)cc1] | + |
| 73 | 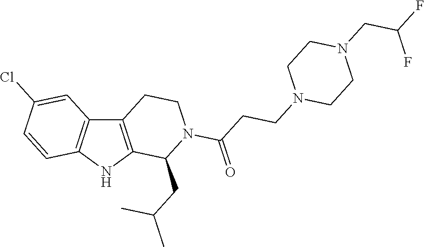 [SMILES: CC(C)C[C@H]1c2[nH]c3ccc(Cl)cc3c2CCN1C(=O)CCN1CCN(CC(F)F)CC1] | ++ |
| 74 | 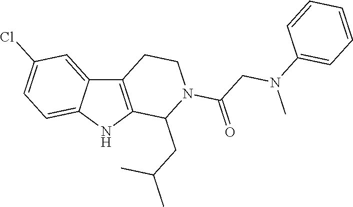 [SMILES: CC(C)CC1c2[nH]c3ccc(Cl)cc3c2CCN1C(=O)CN(C)c1ccccc1] | ++ |
| 75 | 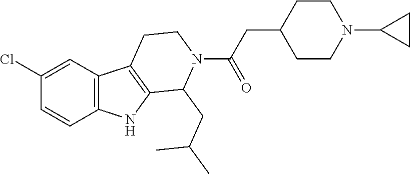 [SMILES: CC(C)CC1c2[nH]c3ccc(Cl)cc3c2CCN1C(=O)CC1CCN(C2CC2)CC1] | ++ |
| 76 | 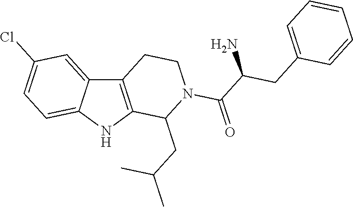 [SMILES: CC(C)CC1c2[nH]c3ccc(Cl)cc3c2CCN1C(=O)[C@@H](N)Cc1ccccc1] | + |
| 77 | 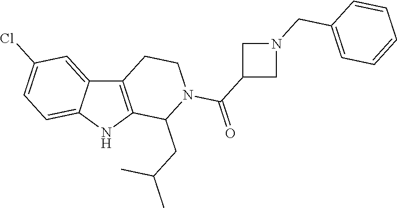 [SMILES: CC(C)CC1c2[nH]c3ccc(Cl)cc3c2CCN1C(=O)C1CN(Cc2ccccc2)C1] | |
| 78 | 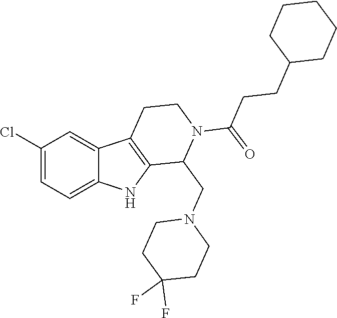 [SMILES: O=C(CCC1CCCCC1)N1CCc2c([nH]c3ccc(Cl)cc23)C1CN1CCC(F)(F)CC1] | ++ |
| 79 | 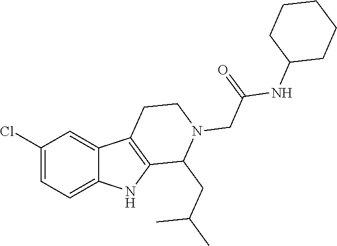 [SMILES: CC(C)CC1c2[nH]c3ccc(Cl)cc3c2CCN1CC(=O)NC1CCCCC1] | ++ |
| 80 | 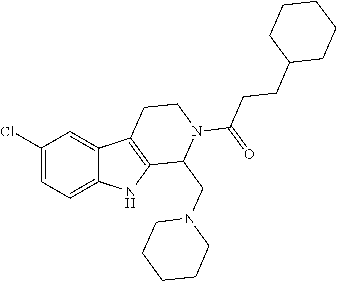 [SMILES: O=C(CCC1CCCCC1)N1CCc2c([nH]c3ccc(Cl)cc23)C1CN1CCCCC1] | + |
| 81 | 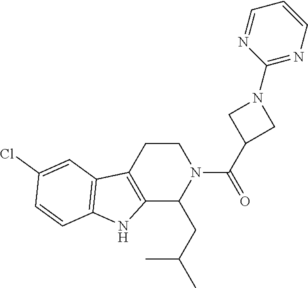 [SMILES: CC(C)CC1c2[nH]c3ccc(Cl)cc3c2CCN1C(=O)C1CN(c2ncccn2)C1] | ++ |
| 82 | 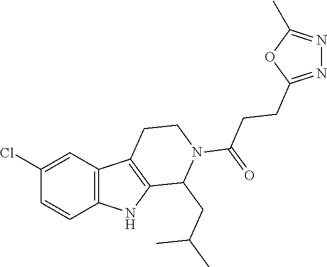 [SMILES: Cc1nnc(CCC(=O)N2CCc3c([nH]c4ccc(Cl)cc34)C2CC(C)C)o1] | ++ |
| 83 | 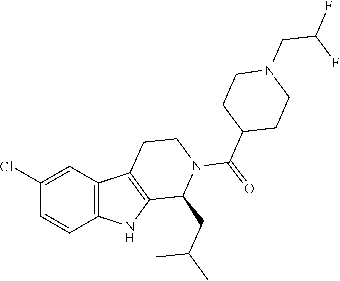 [SMILES: CC(C)C[C@H]1c2[nH]c3ccc(Cl)cc3c2CCN1C(=O)C1CCN(CC(F)F)CC1] | ++ |
| 84 | 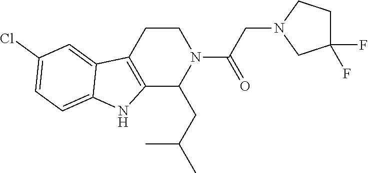 [SMILES: CC(C)CC1c2[nH]c3ccc(Cl)cc3c2CCN1C(=O)CN1CCC(F)(F)C1] | ++ |
| 85 | 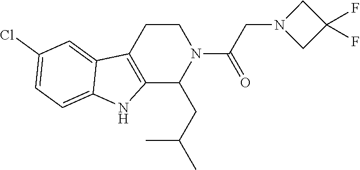 [SMILES: CC(C)CC1c2[nH]c3ccc(Cl)cc3c2CCN1C(=O)CN1CC(F)(F)C1] | +++ |
| 86 | 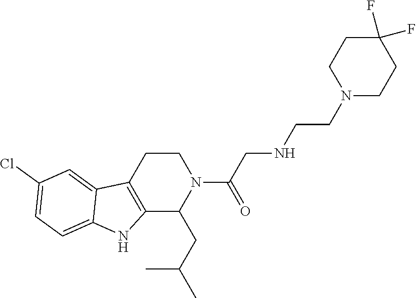 [SMILES: CC(C)CC1c2[nH]c3ccc(Cl)cc3c2CCN1C(=O)CNCCN1CCC(F)(F)CC1] | + |
| 87 | 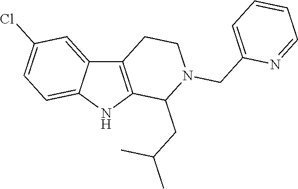 [SMILES: CC(C)CC1c2[nH]c3ccc(Cl)cc3c2CCN1Cc1ccccn1] | ++ |
| 88 | 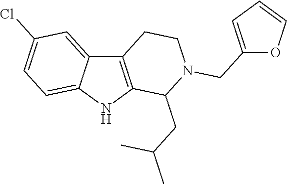 [SMILES: CC(C)CC1c2[nH]c3ccc(Cl)cc3c2CCN1Cc1ccco1] | ++ |
| 89 | 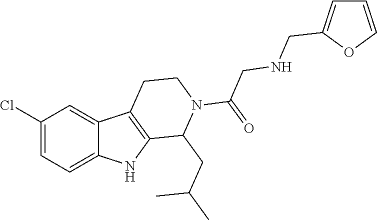 [SMILES: CC(C)CC1c2[nH]c3ccc(Cl)cc3c2CCN1C(=O)CNCc1ccco1] | + |
| 90 | 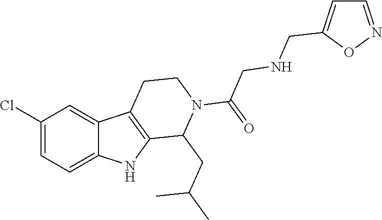 [SMILES: CC(C)CC1c2[nH]c3ccc(Cl)cc3c2CCN1C(=O)CNCc1ccno1] | + |
| 91 | 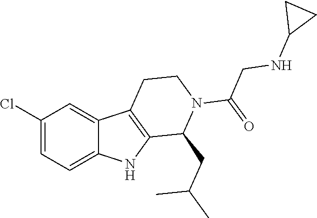 [SMILES: CC(C)C[C@H]1c2[nH]c3ccc(Cl)cc3c2CCN1C(=O)CNC1CC1] | +++ |
| 92 | 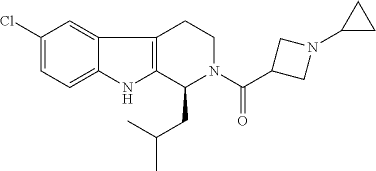 [SMILES: CC(C)C[C@H]1c2[nH]c3ccc(Cl)cc3c2CCN1C(=O)C1CN(C2CC2)C1] | ++ |
| 93 | 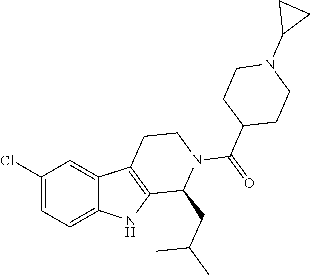 [SMILES: CC(C)C[C@H]1c2[nH]c3ccc(Cl)cc3c2CCN1C(=O)C1CCN(C2CC2)CC1] | ++ |
| 94 | 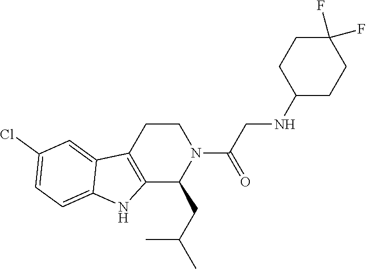 [SMILES: CC(C)C[C@H]1c2[nH]c3ccc(Cl)cc3c2CCN1C(=O)CNC1CCC(F)(F)CC1] | +++ |
| 95 | 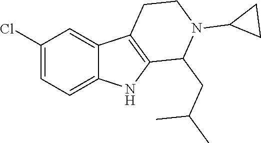 [SMILES: CC(C)CC1c2[nH]c3ccc(Cl)cc3c2CCN1C1CC1] | +++ |
| 96 | 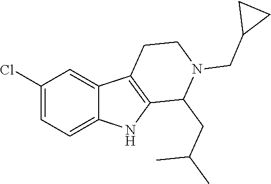 [SMILES: CC(C)CC1c2[nH]c3ccc(Cl)cc3c2CCN1CC1CC1] | ++ |
| 97 | 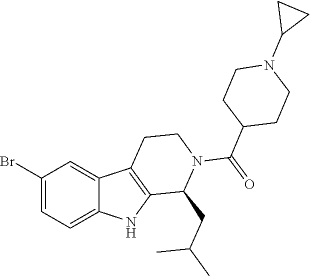 [SMILES: CC(C)C[C@H]1c2[nH]c3ccc(Br)cc3c2CCN1C(=O)C1CCN(C2CC2)CC1] | +++ |
| 98 | 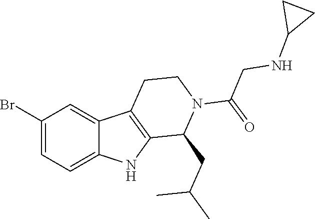 [SMILES: CC(C)C[C@H]1c2[nH]c3ccc(Br)cc3c2CCN1C(=O)CNC1CC1] | +++ |
| 99 | 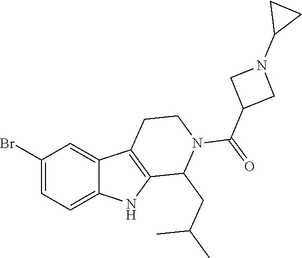 [SMILES: CC(C)CC1c2[nH]c3ccc(Br)cc3c2CCN1C(=O)C1CN(C2CC2)C1] | ++ |
| 100 | 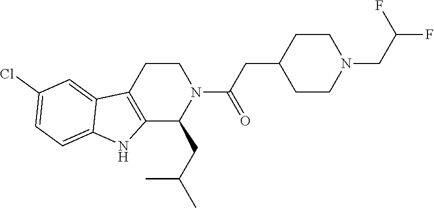 [SMILES: CC(C)C[C@H]1c2[nH]c3ccc(Cl)cc3c2CCN1C(=O)CC1CCN(CC(F)F)CC1] | +++ |
| 101 | 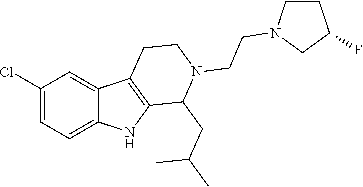 [SMILES: CC(C)CC1c2[nH]c3ccc(Cl)cc3c2CCN1CCN1CC[C@H](F)C1] | + |
| 102 | 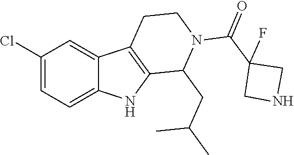 [SMILES: CC(C)CC1c2[nH]c3ccc(Cl)cc3c2CCN1C(=O)C1(F)CNC1] | ++ |
| 103 | 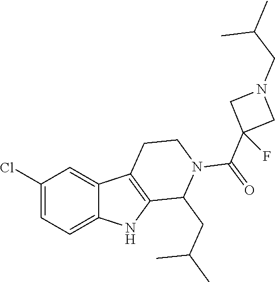 [SMILES: CC(C)CC1c2[nH]c3ccc(Cl)cc3c2CCN1C(=O)C1(F)CN(CC(C)C)C1] | +++ |
| 104 | 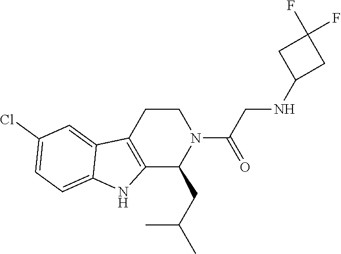 [SMILES: CC(C)C[C@H]1c2[nH]c3ccc(Cl)cc3c2CCN1C(=O)CNC1CC(F)(F)C1] | +++ |
| 105 | 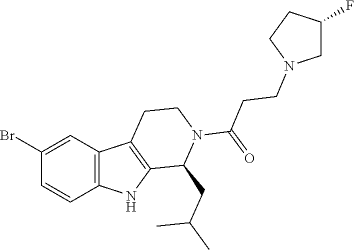 [SMILES: CC(C)C[C@H]1c2[nH]c3ccc(Br)cc3c2CCN1C(=O)CCN1CC[C@H](F)C1] | ++ |
| 106 | 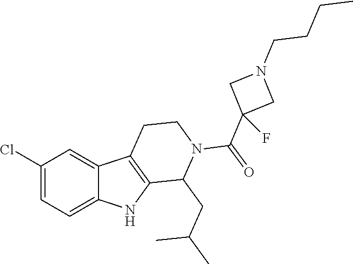 [SMILES: CCCCN1CC(F)(C(=O)N2CCc3c([nH]c4ccc(Cl)cc34)C2CC(C)C)C1] | +++ |
| 107 | 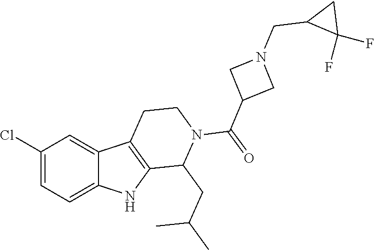 [SMILES: CC(C)CC1c2[nH]c3ccc(Cl)cc3c2CCN1C(=O)C1CN(CC2CC2(F)F)C1] | + |
| 108 | 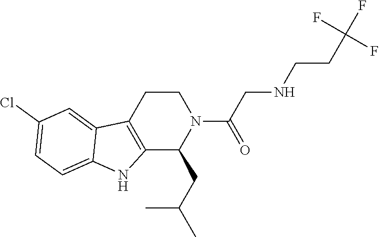 [SMILES: CC(C)C[C@H]1c2[nH]c3ccc(Cl)cc3c2CCN1C(=O)CNCCC(F)(F)F] | +++ |
| 109 | 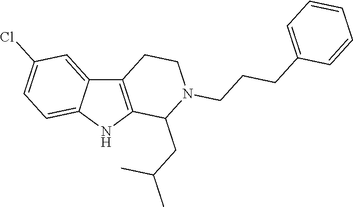 [SMILES: CC(C)CC1c2[nH]c3ccc(Cl)cc3c2CCN1CCCc1ccccc1] | ++ |
| 110 | 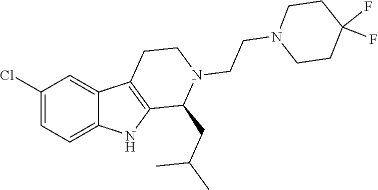 [SMILES: CC(C)C[C@H]1c2[nH]c3ccc(Cl)cc3c2CCN1CCN1CCC(F)(F)CC1] | ++ |

+++ = EC₅₀ < 0.1 μM
++ = EC₅₀ > 0.1 μM but <1.0 μM
+ = EC₅₀ > 1.0 μM

### Example 3

Compounds of the present invention were tested for potency against AdV serotypes Ad3, Ad4, Ad5, Ad7a, Ad8, Ad19, and Ad37 in a plaque reduction assay, a cell-based assay of AdV replication. Ad8, Ad19, and Ad37 are the serotypes most commonly associated with epidemic keratoconjunctivitis (EKC). Ad3, Ad4, and Ad7a are serotypes most commonly associated with follicular conjunctivitis and serotype Ad5 can replicate in the NZW rabbit model of adenoviral ocular keratitis. Ad3, Ad4, Ad5, Ad7a, Ad8, and Ad19 were recovered at the Charles T. Campbell Ophthalmic Microbiology Laboratory from patients presenting with typical adenoviral ocular disease. No clinical isolates of Ad37 were available, so the ATCC (American Type Culture Collection, Manassas, Va.) reference strain of Ad37 was used.

EC₅₀ values (μg/ml) for inhibition of adenoviral replication were determined by plaque reduction assay. Briefly, cultured A549 cells were inoculated with approximately 100 pfu of the indicated adenovirus serotype. Inoculated cells were overlaid with methylcellulose medium containing Compound 2 at 100, 10, 1, 0.1, 0.01, and 0.001 μg/ml, and incubated at 37° C./5% CO₂ for approximately 7 d. Plates were stained with gentian violet, and plaques were counted in a 25× dissecting microscope. IC₅₀, the concentration that inhibited plaque formation by 50%, was estimated from plots of mean plaque count vs. concentration.

Summarized in Table 3, the tested compounds of the present invention exhibited significant antiviral activity against all 7 AdV serotypes tested.

TABLE 3
Adenovirus Serotype EC₅₀ (μg/mL)

| Compound # | Ad3 | Ad4 | Ad5 | Ad7a | Ad8 | Ad19 | Ad37 |
|---|---|---|---|---|---|---|---|
| 6 | +++ | ++ | +++ | +++ | +++ | +++ | +++ |
| 35 | ++ | + | ++ | ++ | ++ | ++ | ++ |
| 43 | ++ | ++ | +++ | +++ | +++ | +++ | +++ |
| 47 | ++ | ++ | ++ | ++ | ++ | ++ | ++ |
| 73 | ++ | ++ | ++ | ++ | ++ | + | + |
| 91 | ++ | ++ | +++ | ++ | ++ | ++ | ++ |
| 92 | + | + | ++ | ++ | + | ++ | ++ |
| 93 | ++ | ++ | ++ | ++ | ++ | ++ | ++ |
| 97 | ++ | + | +++ | ++ | +++ | ++ | ++ |

+++ = EC₅₀ < 0.1 μg/mL
++ = EC₅₀ > 0.1 μg/mL but < 1.0 μg/mL
+ = EC₅₀ > 1.0 μg/mL

### Example 4

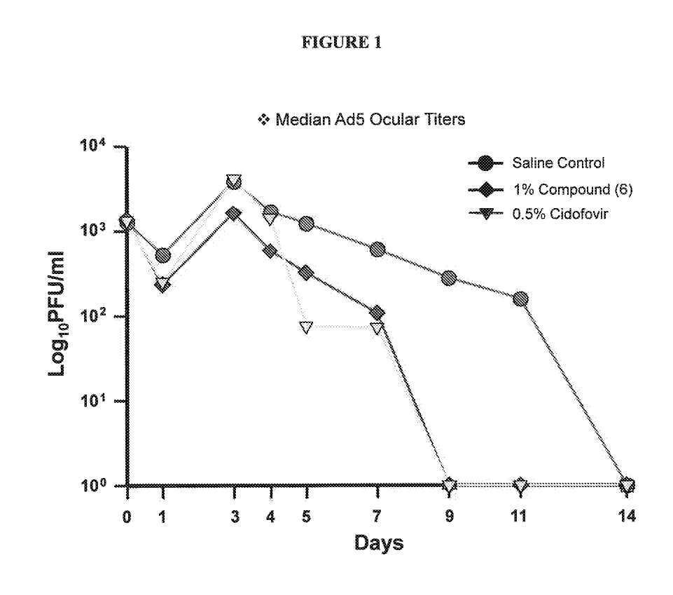

Compound 6 exhibited significant antiviral efficacy in a rabbit model of adenoviral (Ad5) ocular keratitis. These results are presented in FIG. 1. New Zealand White rabbits were anesthetized, and corneas were scarified (12 cross-hatched strokes of a #25 needle) and inoculated with 50 μl of 3.0×10⁷ pfu/ml (1.5×10⁶ pfu/eye) of Ad5. Compound 6 (1%) was formulated in a sodium acetate buffer solution at pH 4.5 with 2% PEG 8000, 1.0% polysorbate 80 and 0.5% HPMC. Rabbits were dosed topically 8 times a day for 9d, with at least 45 min between doses. Saline and 0.5% Cidofovir were negative and positive controls, respectively. Eyes were cultured for Ad5 on days 0, 1, 3, 4, 5, 7, 9, 11, and 14 at least 1 h following the final dose. Rabbit eyes were anesthetized topically, and a cotton-tipped swab was placed into the lower formix of each eye and rolled over the cornea into the upper formix to recover adenovirus from the tear film and corneal and conjunctival surfaces. The swabs from each eye were placed individually into tubes containing 1 ml of outgrowth media, and were frozen at −70° C. Cultures were titered by plaque formation assay in A549 cells. Data are presented as median daily ocular titers. Compound 6 demonstrated significant antiviral activity that persisted throughout treatment. Compound 6 reduced median viral titer significantly relative to the saline control from days 2-11.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

What is claimed is:

1. An ophthalmic pharmaceutical composition useful in the treatment of ocular infection, comprising an effective amount of a compound according to Formula I, or a pharmaceutically acceptable salt thereof: 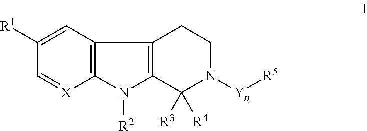
where:
X=C or N;
Yₙ=bond, CH₂, C(O), C(O)O, C(O)NR⁶, or SO₂;
n=0 or 1;
p=0, 1 or 2;
R¹=H, halogen, alkyl, nitrile or amide;
R²=H or alkyl;
R³ and R⁴ are independently selected from H, alkyl, cycloalkyl, heteroalkyl, heterocycloalkyl, aryl, or heteroaryl, or R³ and R⁴ can form a 3- to 6-membered cycloalkyl or heterocycloalkyl ring optionally substituted with alkyl or halogen;
R⁵=R⁷N(R⁸)R⁹, R⁷C(O)N(R⁸)R⁹, or R⁷SO₂R¹⁰
R⁶=H or alkyl;
R⁷=null or optionally substituted alkylene;
R⁸ and R⁹ are independently selected from H, alkyl, heteroalkyl, cycloalkyl, heterocycloalkyl, aryl, or heteroaryl each of which can be optionally substituted or R⁸ can combine with R⁷ and/or R⁹ to form a 3- to 8-membered heterocycle optionally substituted with halogen, alkyl, cyano, NR¹⁰, and/or S(O)ₚ; and
R¹⁰=H or alkyl.

2. The composition of claim 1, further comprising a compound selected from the group consisting of:
ophthalmologically acceptable preservatives, surfactants, viscosity enhancers, penetration enhancers, gelling agents, hydrophobic bases, vehicles, buffers, sodium chloride, and water.

3. The composition of claim 1, wherein said composition further comprises a antiinfective or antiinflammatory agent in addition to a compound of Formula I.

4. The composition of claim 3, wherein the antiinfective or antiinflammatory agent is selected from the group consisting of:
steroidal antiinflammatories, nonsteroidal inflammatories, nepafenac, dexamethasone, and combinations thereof.

5. The composition of claim 1 wherein said composition comprises from about 0.01 percent weight/volume to about 5 percent weight/volume of said compound.

6. A method of treating ocular infection, which comprises administering to a human or other mammal a therapeutically effective amount of a pharmaceutical composition comprising a compound according to Formula I, or a pharmaceutically acceptable salt thereof: 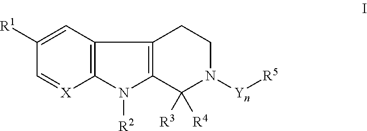
where:
X=C or N;
Yₙ=bond, CH₂, C(O), C(O)O, C(O)NR⁶, or SO₂;
n=0 or 1;
p=0, 1 or 2;
R¹=H, halogen, alkyl, nitrile or amide;
R²=H or alkyl;
R³ and R⁴ are independently selected from H, alkyl, cycloalkyl, heteroalkyl, heterocycloalkyl, aryl, or heteroaryl, or R³ and R⁴ can form a 3- to 6-membered cycloalkyl or heterocycloalkyl ring optionally substituted with alkyl or halogen;
R⁵=R⁷N(R⁸)R⁹, R⁷C(O)N(R⁸)R⁹, or R⁷SO₂R¹⁰
R⁶═H or alkyl;
R⁷=null or optionally substituted alkylene;
R⁸ and R⁹ are independently selected from H, alkyl, heteroalkyl, cycloalkyl, heterocycloalkyl, aryl, or heteroaryl each of which can be optionally substituted or R⁸ can combine with R⁷ and/or R⁹ to form a 3- to 8-membered heterocycle optionally substituted with halogen, alkyl, cyano, NR¹⁰, and/or S(O)ₚ; and
R¹⁰=H or alkyl;
and a pharmaceutically acceptable vehicle therefore.

7. The method of claim 6 wherein said administering comprises applying 1 to 2 drops of a composition comprising from about 0.01 percent weight/volume to about 5 percent weight/volume of compound according to Formula II to 4 times daily.

8. The method of claim 7 wherein said composition further comprises a antiinfective or antiinflammatory treatment agent in addition to a compound of Formula I.

9. The method of claim 8, wherein the antiinfective or antiinflammatory agent is selected from the group consisting of:
steroidal antiinflammatories, nonsteroidal inflammatories, nepafenac, dexamethasone, and combinations thereof.

10. The method of claim 7 wherein said composition comprises from about 0.1 percent weight/volume to about 1 percent weight/volume of said compound.

11. An ophthalmic pharmaceutical composition useful in the treatment of ocular infection, comprising an effective amount of a compound according to Formula II, or a pharmaceutically acceptable salt thereof: 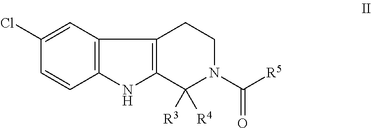
where:
R³ and R⁴ are independently selected from H, alkyl, cycloalkyl, heteroalkyl, heterocycloalkyl, aryl, or heteroaryl, or R³ and R⁴ can form a 3- to 6-membered cycloalkyl or heterocycloalkyl ring optionally substituted with alkyl or halogen;
R⁵=R⁷N(R⁸)R⁹, R⁷C(O)N(R⁸)R⁹, or R⁷SO₂R¹⁰
R⁷=optionally substituted alkylene;
R⁸ and R⁹ are independently selected from H, alkyl, heteroalkyl, cycloalkyl, heterocycloalkyl or heteroaryl each of which can be optionally substituted or R⁸ can combine with R⁷ and/or R⁹ to form a 4- to 8-membered heterocycle optionally substituted with halogen, alkyl, NR¹⁰, N(O) and/or S(O)ₚ; and
R¹⁰=H or alkyl;
and a pharmaceutically acceptable vehicle therefore.

12. The composition of claim 11, further comprising a compound selected from the group consisting of:
ophthalmologically acceptable preservatives, surfactants, viscosity enhancers, penetration enhancers, gelling agents, hydrophobic bases, vehicles, buffers, sodium chloride, and water.

13. The composition of claim 11, wherein said composition further comprises a antiinfective or antiinflammatory agent in addition to a compound of Formula II, said agent selected from the group consisting of:
steroidal antiinflammatories, nonsteroidal inflammatories, nepafenac, dexamethasone, and combinations thereof.

14. The composition of claim 11 wherein said composition comprises from about 0.01 percent weight/volume to about 5 percent weight/volume of said compound.

15. A method of treating ocular infection, which comprises administering to a human or other mammal a therapeutically effective amount of a pharmaceutical composition comprising a compound according to Formula II, or a pharmaceutically acceptable salt thereof.

16. An ophthalmic pharmaceutical composition useful in the treatment of ocular infection, comprising an effective amount of a compound according to Formula III, or a pharmaceutically acceptable salt thereof: 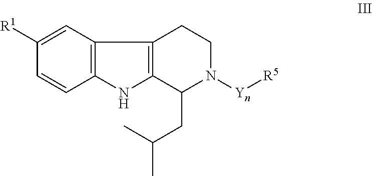
where:
R¹=Cl or Br
Yₙ=bond, CH₂ or C(O);
R⁵=R⁷N(R⁸)R⁹, R⁷C(O)N(R⁸)R⁹, or R⁷SO₂R¹⁰
R⁷=null or optionally substituted alkylene;
R⁸ and R⁹ are independently selected from H, alkyl, alkylene, heteroalkyl, cycloalkyl, heterocycloalkyl or heteroaryl each of which can be optionally substituted or R⁸ can combine with R⁷ and/or R⁹ to form a 4- to 8-membered heterocycle optionally substituted with halogen, alkyl, NR¹⁰, and/or S(O)ₚ; and
R¹⁰=H or optionally substituted alkyl;
and a pharmaceutically acceptable vehicle therefore.

17. A method of treating ocular infection, which comprises administering to a human or other mammal a therapeutically effective amount of a pharmaceutical composition comprising a compound according to Formula III, or a pharmaceutically acceptable salt thereof.

18. A compound selected from the group consisting of Compounds 1-110.

19. A compound of Formula I: 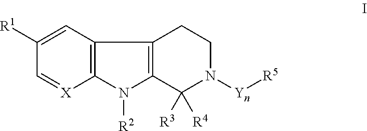
where:
X=C or N;
Yₙ=CH₂, C(O), C(O)O, C(O)NR⁶, or SO₂;
n=0 or 1;
p=0, 1 or 2;
R¹=H, halogen, alkyl, nitrile or amide;
R²=H or alkyl;
R³ and R⁴ are independently selected from H, alkyl, cycloalkyl, heteroalkyl, heterocycloalkyl, aryl, or heteroaryl, or R³ and R⁴ can form a 3- to 6-membered cycloalkyl or heterocycloalkyl ring optionally substituted with alkyl or halogen;
R⁵═R⁷N(R⁸)R⁹, R⁷C(O)N(R⁸)R⁹, or R⁷SO₂R¹⁰
R⁶═H or alkyl;
R⁷=null or optionally substituted alkylene;
R⁸ and R⁹ are independently selected from H, alkyl, heteroalkyl, cycloalkyl, heterocycloalkyl, aryl, or heteroaryl each of which can be optionally substituted or R⁸ can combine with R⁷ and/or R⁹ to form a 3- to 8-membered heterocycle optionally substituted with halogen, alkyl, cyano, NR¹⁰, and/or S(O)ₚ; and
R¹⁰=H or alkyl.
